(19) United States
(12) Patent Application Publication
(10) Pub. No.: US 2026/0098043 A1
(43) Pub. Date: Apr. 9, 2026

(54) POLYCYCLIC COMPOUND AND USE THEREOF

(71) Applicant: HAINAN SIMCERE ZAIMING PHARMECEUTICAL CO., LTD., Hainan (CN)

(72) Inventors: Jinghai JIN, Shanghai (CN); Yuewen LI, Shanghai (CN); Yu SHI, Shanghai (CN); Wei ZHU, Shanghai (CN); Zhengtao LI, Shanghai (CN)

(21) Appl. No.: 19/111,594

(22) PCT Filed: Sep. 13, 2023
(86) PCT No.: PCT/CN2023/118650

§ 371 Date: Mar. 13, 2025

(30) Foreign Application Priority Data
Sep. 14, 2022 (CN) 202211119510.1
Sep. 20, 2022 (CN) 202211141718.3
Nov. 24, 2022 (CN) 202211484237.2

(51) Int. Cl. C07D 495/14; A61K 31/454; A61K 31/5377; A61K 31/5517; A61K 31/553; A61K 31/675; A61P 35/00; C07D 401/04; C07D 401/14; C07D 413/04; C07D 413/14; C07D 417/14; C07D 487/14; C07D 487/16; C07D 498/04; C07D 498/08; C07D 519/00; C07F 9/6558

(52) U.S. Cl. CPC C07D 495/14; A61K 31/454; A61K 31/5377; A61K 31/5517; A61K 31/553; A61K 31/675; A61P 35/00; C07D 401/04; C07D 401/14; C07D 413/04; C07D 413/14; C07D 417/14; C07D 487/14; C07D 487/16; C07D 498/04; C07D 498/08; C07D 519/00; C07F 9/65583

(57) ABSTRACT

A compound represented by formula (I) CLM-L-PTM or a pharmaceutically acceptable salt thereof, a pharmaceutical composition containing same, and a use thereof, which are particularly suitable for preparing drugs for treating or preventing abnormal cell proliferation diseases.

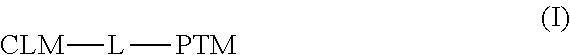

The present disclosure claims priority to Patent Application No. 202211119510.1 entitled “POLYCYCLIC COMPOUND, PHARMACEUTICAL COMPOSITION AND USE THEREOF”, filed with China National Intellectual Property Administration on Sep. 14, 2022; Patent Application No. 202211141718.3 entitled “POLYCYCLIC COMPOUND AND USE THEREOF”, filed with China National Intellectual Property Administration on Sep. 20, 2022; and Patent Application No. 202211484237.2 entitled “POLYCYCLIC COMPOUND AND USE THEREOF”, filed with China National Intellectual Property Administration on Nov. 24, 2022, which are incorporated herein by reference in their entirety.

### TECHNICAL FIELD

The present disclosure relates to the technical field of medicines, and particularly to a proteolysis targeting chimera (PROTAC) compound containing a polycyclic cereblon E3 ubiquitin ligase ligand, or a pharmaceutically acceptable salt thereof, which has biological activities such as antiproliferative activity for tumor cells and can be used for treating related diseases.

### BACKGROUND

CRBN (cereblon) is a protein encoded by the CRBN gene in the human body. CRBN is widely expressed in the testis, spleen, prostate, liver, pancreas, placenta, kidney, lung, skeletal muscle, ovary, small intestine, peripheral blood leukocytes, colon, brain, and retina, while its expression in brain tissues (including the retina) and testis is significantly higher than that in other tissues.

CRBN is an important target of anti-tumor and immunomodulator drugs, and CRBN-targeting drugs have been proven to have definite therapeutic effects on various hematologic malignancies such as multiple myeloma and chronic lymphocytic leukemia and autoimmune diseases such as systemic lupus erythematosus. However, the existing-domide drugs have many side effects, especially peripheral neuropathy. Currently, there is a need to develop new CRBN modulator drugs to improve clinical therapeutic effects, reduce clinical side effects, and facilitate long-term use by patients.

The ubiquitin-proteasome pathway (UPP) is a key pathway for the regulation of key regulatory proteins and the degradation of misfolded or abnormal proteins. Ubiquitin molecules are covalently linked to the terminal lysine residues via E3 ubiquitin ligases to tag proteins for proteasomal degradation, where the proteins are digested into small peptides and ultimately into their constituent amino acids, which are used as building blocks for new proteins. UPP is important for a variety of cellular processes, and its defects or imbalances can lead to the pathogenesis of various diseases. Defective proteasomal degradation has been confirmed to be associated with a variety of clinical disorders, including Alzheimer's disease, Parkinson's disease, Huntington's disease, muscular dystrophy, cardiovascular diseases, cancers, etc. Proteolysis targeting chimeras (PROTACs) are a novel technology for the targeted degradation of target proteins, developed based on the cell's own ubiquitin-proteasome system (UPS). The PROTAC molecule is a bifunctional molecule that can both binds to a target protein and recruits E3 ubiquitin ligases, thereby ubiquitinating the target protein and subsequently degrading it via the proteasome. Thus, CRBN ligands can also be used to prepare bifunctional PROTAC compounds for treating related diseases.

### SUMMARY

The present disclosure provides a compound represented by formula (I) or a pharmaceutically acceptable salt thereof:

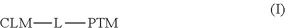

wherein
CLM has the structure shown below:

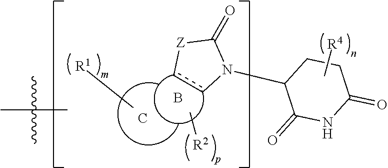

“ [symbol] ” is selected from a single bond and a double bond;
Z is selected from C(R³)₂, NR³, and O;
ring B is selected from 5- to 6-membered heteroaromatic ring, 5- to 8-membered heterocyclic ring, benzene ring, and C₅-C₈ saturated or partially saturated carbon ring;
ring C is selected from 5- to 6-membered heteroaromatic ring, 5- to 8-membered heterocyclic ring, benzene ring, and C₅-C₈ saturated or partially saturated carbon ring;
R¹, R², and R⁵ are each independently selected from halogen, ═O, CN, NO₂, —OR, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
each R³ is independently selected from H, halogen, ═O, CN, NO₂, —OR, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
or R¹ and R³, together with the atoms linked thereto, form C₅-C₈ saturated or partially saturated carbon ring, 5- to 6-membered heteroaromatic ring, or 5- to 8-membered heterocyclic ring, wherein the C₅-C₈ saturated or partially saturated carbon ring, 5- to 6-membered heteroaromatic ring, or 5- to 8-membered heterocyclic ring is optionally substituted with R⁵;
each R⁴ is independently selected from halogen, CN, NO₂, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₂-C₁₀ alkenyl, C₂-C₁₀ alkynyl, C₃-C₁₀ cycloalkyl, and 4- to 8-membered heterocyclyl, wherein the 2- to 10-membered heteroalkyl, C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and p are independently selected from 0, 1, 2, 3, 4, 5, and 6;
L represents a linking unit of CLM and PTM;
PTM is selected from binding moieties of targeted proteins.

In some embodiments, Z is selected from NR³ and O.

In some embodiments, Z is NR³.

In some embodiments, Z is NCH₃.

In some embodiments, ring B is selected from 5- to 6-membered heteroaromatic ring, 5- to 6-membered heterocyclic ring, benzene ring, and C₅-C₆ saturated or partially saturated carbon ring.

In some embodiments, ring B is selected from 5- to 6-membered heteroaromatic ring and benzene ring.

In some embodiments, ring B is selected from pyridine ring and benzene ring. In some embodiments, ring C is selected from 5- to 6-membered heteroaromatic ring, 5- to 6-membered heterocyclic ring, benzene ring, and C₅-C₆ saturated or partially saturated carbon ring.

In some embodiments, ring C is selected from 5- to 6-membered heteroaromatic ring and benzene ring.

In some embodiments, ring C is selected from benzene ring, pyridine ring, pyrrole ring, and thiazole ring.

In some embodiments, ring C is selected from benzene ring and pyridine ring.

In some embodiments, ring C is benzene ring.

In some embodiments, CLM is selected from the structure represented by formula (II):

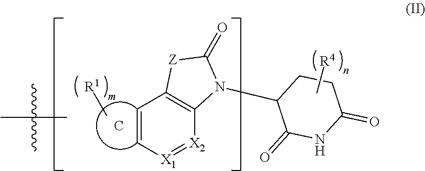

wherein X₁ and X₂ are independently selected from N and CH, wherein the CH is optionally substituted with R²; ring C, Z, R¹, R², R⁴, m, and n are as defined above.

In some embodiments, CLM is selected from structures represented by formulas (II-1a) and (II-1b):

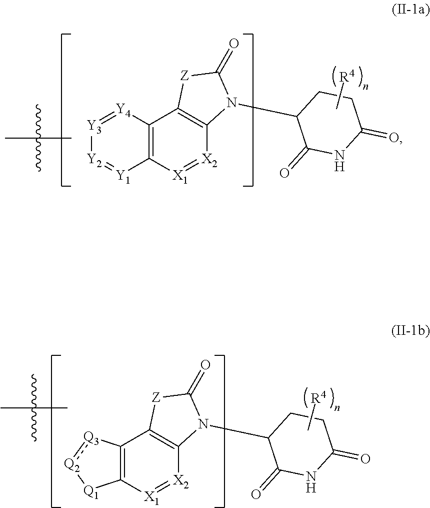

wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a single bond and a double bond; Q₁, Q₂, and Q₃ are independently selected from O, S, NH, CH₂, N, and CH, wherein the NH, CH₂, or CH is optionally substituted with R¹; Z, R¹, R², R⁴, and n are as defined above.

In some embodiments, “ [symbol] ” is selected from a double bond, Q₁ is selected from O, S, NH, and CH₂, and Q₂ and Q₃ are indepndentlysTelected from N and CH, wherein the NH, CH₂, or CH is optionally substituted with R¹.

In some embodiments, R¹ and R² are independently selected from halogen, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}.

In some embodiments, R³ is selected from halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a}.

In some embodiments, each R³ is independently selected from H, halogen, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}.

In some embodiments, each R³ is independently selected from H, C₁-C₁₀ alkyl, and C₃-C₁₀ cycloalkyl, wherein the C₁-C₁₀ alkyl or C₃-C₁₀ cycloalkyl is optionally substituted with R^{a}.

In some embodiments, each R³ is independently selected from C₁-C₆ alkyl, wherein the C₁-C₆ alkyl is optionally substituted with R^{a}.

In some embodiments, each R³ is independently selected from CH₃.

In some embodiments, R¹ and R³, together with the atoms linked thereto, form 5-, 6-, 7-, or 8-membered heterocyclic ring, wherein the 5-, 6-, 7-, or 8-membered heterocyclic ring is optionally substituted with R⁵.

In some embodiments, R¹ and R³, together with the atoms linked thereto, form 5-, 6-, 7-, or 8-membered heterocyclic ring, wherein the 5-, 6-, 7-, or 8-membered heterocyclic ring contains 1, 2, or 3 heteroatoms or heteroatom groups selected from N, O, and S, and the 5-, 6-, 7-, or 8-membered heterocyclic ring is optionally substituted with R⁵.

In some embodiments, each R⁵ is independently selected from halogen, ═O, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}.

In some embodiments, each R⁵ is independently selected from ═O, C₁-C₁₀ alkyl, and C₃-C₁₀ cycloalkyl, wherein the C₁-C₁₀ alkyl or C₃-C₁₀ cycloalkyl is optionally substituted with R^{a}.

In some embodiments, each R⁵ is independently selected from ═O and C₁-C₆ alkyl, wherein the C₁-C₆ alkyl is optionally substituted with R^{a}.

In some embodiments, each R⁵ is C₁-C₆ alkyl, wherein the C₁-C₆ alkyl is optionally substituted with R^{a}.

In some embodiments, R⁵ is CH₃.

In some embodiments, each R⁴ is independently selected from halogen, CN, OH, NH₂, and C₁-C₆ alkyl, wherein the OH, NH₂, or C₁-C₆ alkyl is optionally substituted with R^{a}.

In some embodiments, each R^{a} is independently selected from halogen, CN, OH, NH₂, C₁-C₁₀ alkyl, C₂-C₁₀ alkenyl, C₂-C₁₀ alkynyl, C₃-C₁₀ cycloalkyl, and 4- to 8-membered heterocyclyl, wherein the C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c}.

In some embodiments, each R^{a} is independently selected from halogen, CN, OH, NH₂ or C₁-C₆ alkyl, C₂-C₆ alkenyl, and C₂-C₆ alkynyl, wherein the C₁-C₆ alkyl, C₂-C₆ alkenyl, or C₂-C₆ alkynyl is optionally substituted with R^{c}.

In some embodiments, each R^{a} is independently selected from halogen and C₁-C₆ alkyl, wherein the C₁-C₆ alkyl is optionally substituted with R^{c}.

In some embodiments, each R^{a} is independently selected from F, Cl, and CH₃.

In some embodiments, each R^{b} is independently selected from H and C₁-C₆ alkyl, wherein the C₁-C₆ alkyl is optionally substituted with R^{c}.

In some embodiments, each R^{c} is independently selected from halogen, CN, OH, NH₂, and C₁-C₆ alkyl.

In some embodiments, m and p are independently selected from 0, 1, 2, 3, and 4.

In some embodiments, m and p are independently selected from 0, 1, and 2.

In some embodiments, m and p are independently selected from 1 and 2.

In some embodiments, m and p are both 0.

In some embodiments, n is selected from 0 and 1.

In some embodiments, n is 0.

In some embodiments, L is selected from

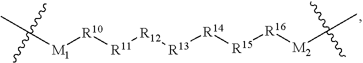

wherein M₁ and M₂ are independently selected from bond, —NR²⁰—, —C(O)—, —C(O)O—, —SO₂—, —S(O)—, —O—, —S—, —C(═S)—, —C(O)NR²⁰—, —NR²⁰C(O)O—, —NR²⁰S(O)₂—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹;
R¹⁰, R¹¹, R¹², R¹³, R¹⁴, R¹⁵, and R¹⁶ are independently selected from bond, —(O—CH₂CH₂)ₖ—, —C(O)—, —C(O)O—, —SO₂—, —S(O)—, —O—, —S—, —C(S)—, —C(═NR²⁰)—, —C(O)NR²⁰—, —NR²⁰—, —NR²⁰C(O)O—, —NR²⁰S(O)₂—, —P(O)R²⁰—, —P(O)(OR²⁰)O—, —P(O)(OR²⁰)—

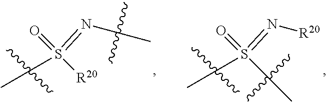

2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹;
k is independently selected from 1, 2, 3, 4, 5, and 6;
R²⁰ is selected from H, halogen, CN, OH, NH₂, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{f};
R²¹ is selected from halogen, CN, OH, NH₂, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{f};
each R is independently selected from halogen, CN, OH, NH₂, and C₁-C₆ alkyl.

In some embodiments, R²⁰ is selected from H and C₁-C₁₀ alkyl, wherein the C₁-C₁₀ alkyl is optionally substituted with R.

In some embodiments, R²¹ is selected from halogen, CN, OH, NH₂, NO₂, and C₁-C₁₀ alkyl, wherein the C₁-C₁₀ alkyl is optionally substituted with R^{f}.

In some embodiments, L is selected from

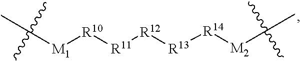

wherein M₁, M₂, R¹⁰, R¹¹, R¹², R¹³, and R¹⁴ are as defined above.

In some embodiments, L is selected from

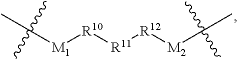

wherein M₁, M₂, R¹⁰, R¹¹, and R¹² are as defined above.

In some embodiments, L is selected from

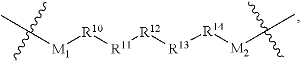

wherein R¹⁰, R¹¹, R¹², R¹³, and R¹⁴ are independently selected from bond, —(O—CH₂CH₂)ₖ—, —C(O)—, —C(O)O—, —O—, —C(O)NR²⁰—, —NR²⁰—, —NR²⁰C(O)O—, —NR²⁰S(O)₂—, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the C₁-C₁₀ alkylene, C₂-C₁₀ alkenylene, C₂-C₁₀ alkynylene, 4- to 9-membered heterocyclylene, C₆-C₁₀ arylene, or 5- to 10-membered heteroarylene is optionally substituted with R²¹; M₁, M₂, R²⁰, R²¹, and k are as defined above.

In some embodiments, L is selected from

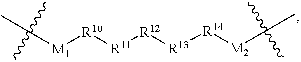

wherein R¹¹, R¹², and R¹³ are independently selected from a bond, —(O—CH₂CH₂)ₖ—, C₁-C₁₀ alkylene, and C₂-C₁₀ alkenylene, wherein the C₁-C₁₀ alkylene or C₂-C₁₀ alkenylene is optionally substituted with R²¹; R¹⁰, R¹⁴, M₁, and M₂ are independently selected from bond, —C(O)—, —C(O)O—, —C(O)NR²⁰—, —O—, —NR²⁰—, —NR²⁰C(O)O—, —NR²⁰S(O)₂—, C₁-C₁₀ alkylene, C₂-C₁₀ alkenylene, C₂-C₁₀ alkynylene, 4- to 9-membered heterocyclylene, and 5- to 10-membered heteroarylene, wherein the C₁-C₁₀ alkylene, C₂-C₁₀ alkenylene, C₂-C₁₀ alkynylene, 4- to 9-membered heterocyclylene, or 5- to 10-membered heteroarylene is optionally substituted with R²¹; wherein k, R²⁰, and R²¹ are as defined above.

In some embodiments, M₁ and M₂ are independently selected from bond, —NR²⁰—, —C(O)—, —C(O)O—, —O—, —S—, —C(O)NR²⁰—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹; wherein R²⁰ and R²¹ are as defined above.

In some embodiments, M₁ and M₂ are independently selected from bond, —NR²⁰—, —C(O)—, —C(O)O—, —O—, —C(O)NR²⁰—, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹.

In some embodiments, R¹⁰, R¹¹, R¹², R¹³, R¹⁴, R¹⁵, and R¹⁶ are independently selected from bond, —(O—CH₂CH₂)ₖ—, —C(O)—, —C(O)O—, —SO₂—, —S(O)—, —O—, —S—, —C(O)NR²⁰—, —NR²⁰—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹, wherein k, R²⁰, and R²¹ are as defined above.

In some embodiments, R¹⁰, R¹¹, R¹², R¹³, R¹⁴, R¹⁵, and R¹⁶ are independently selected from bond, —(O—CH₂CH₂)ₖ—, —C(O)—, —C(O)O—, —O—, —C(O)NR²⁰—, —NR²⁰—, C₁-C₆ alkylene, C₂-C₆ alkenylene, C₂-C₆ alkynylene, C₃-C₆ cycloalkylene, 4- to 9-membered heterocyclylene, C₆-C₁₀ arylene, and 5- to 6-membered heteroarylene, wherein the C₁-C₆ alkylene, C₂-C₆ alkenylene, C₂-C₆ alkynylene, C₃-C₆ cycloalkylene, 4- to 9-membered heterocyclylene, C₆-C₁₀ arylene, or 5- to 6-membered heteroarylene is optionally substituted with R²¹, wherein k, R²⁰, and R²¹ are as defined above.

In some embodiments, L is selected from the following structures:
—C(O)—(O—CH₂CH₂)ₖ—C₁-C₁₀ alkylene-C(O)NR²⁰—,
—NR²⁰—(O—CH₂CH₂)ₖ—C₁-C₁₀ alkylene-C(O)NR²⁰—,
—NR²⁰—(O—CH₂CH₂)ₖ—C₁-C₁₀ alkylene-C(O)—,
—C(O)—C₁-C₁₀ alkylene-C₁-C₁₀ alkylene-,
—C(O)—C₁-C₁₀ alkylene-C₂-C₁₀ alkynylene-,
—C(O)—C₁-C₁₀ alkylene-C₁-C₁₀ alkylene-4- to 9-membered heterocyclylene-,
—C(O)—C₁-C₁₀ alkylene-C₁-C₁₀ alkylene-5- to 10-membered heteroarylene-,
—C(O)—C₁-C₁₀ alkylene-C₁-C₁₀ alkylene-C(O)-4- to 9-membered heterocyclylene-,
—C(O)—C₁-C₁₀ alkylene-C₁-C₁₀ alkylene-C(O)NR²⁰-4- to 9-membered heterocyclylene-,
—C(O)—C₁-C₁₀ alkylene-C₁-C₁₀ alkylene-C(O)O—,
—C(O)—C₁-C₁₀ alkylene-C₂-C₁₀ alkenylene-4- to 9-membered heterocyclylene-O—,
—C(O)—C₁-C₁₀ alkylene-C₁-C₁₀ alkylene-4- to 9-membered heterocyclylene-C(O)—,
—C(O)—C₃-C₁₀ cycloalkylene-C₁-C₁₀ alkylene-O—,
—C(O)—C₃-C₁₀ cycloalkylene-C₁-C₁₀ alkylene-NR²⁰—,
—C(O)—C₃-C₁₀ cycloalkylene-C₁-C₁₀ alkylene-NR²⁰—C₁-C₁₀ alkylene-,
—NR²⁰—C₁-C₁₀ alkylene-C(O)NR²⁰—C₁-C₁₀ alkylene-, and
—C₁-C₁₀ alkylene-4- to 9-membered heterocyclylene-C₁-C₆ alkylene-4- to 9-membered heterocyclylene-,
wherein k and R²⁰ are as defined above.

In some embodiments, L is selected from the following structures:
—C(O)—(O—CH₂CH₂)ₖ—C₁-C₆ alkylene-C(O)NH—,
—NH—(O—CH₂CH₂)ₖ—C₁-C₆ alkylene-C(O)NH—,
—NH—(O—CH₂CH₂)ₖ—C₁-C₆ alkylene-C(O)—,
—C(O)—C₁-C₁₀ alkylene-C₁-C₆ alkylene-,
—C(O)—C₁-C₁₀ alkylene-C₂-C₆ alkynylene-,
—C(O)—C₁-C₁₀ alkylene-C₁-C₆ alkylene-4- to 9-membered heterocyclylene-,
—C(O)—C₁-C₁₀ alkylene-C₁-C₆ alkylene-5- to 6-membered heteroarylene-,
—C(O)—C₁-C₁₀ alkylene-C₁-C₆ alkylene-C(O)-4- to 9-membered heterocyclylene-,
—C(O)—C₁-C₁₀ alkylene-C₁-C₆ alkylene-C(O)NH-4- to 9-membered heterocyclylene-,
—C(O)—C₁-C₁₀ alkylene-C₁-C₆ alkylene-C(O)O—,
—C(O)—C₁-C₆ alkylene-C₂-C₆ alkenylene-4- to 6-membered heterocyclylene-O—,
—C(O)—C₁-C₁₀ alkylene-C₁-C₆ alkylene-4- to 6-membered heterocyclylene-C(O)—,
—C(O)—C₃-C₆ cycloalkylene-C₁-C₆ alkylene-O—,
—C(O)—C₃-C₆ cycloalkylene-C₁-C₆ alkylene-NH—,
—C(O)—C₃-C₆ cycloalkylene-C₁-C₆ alkylene-NH—C₁-C₆ alkylene-,
—NH—C₁-C₆ alkylene-C(O)NH—C₁-C₆ alkylene-, and
—C₁-C₆ alkylene-4- to 6-membered heterocyclylene-C₁-C₆ alkylene-4- to 6-membered heterocyclylene-,
wherein k is as defined above.

In some embodiments, L is selected from the following structures:

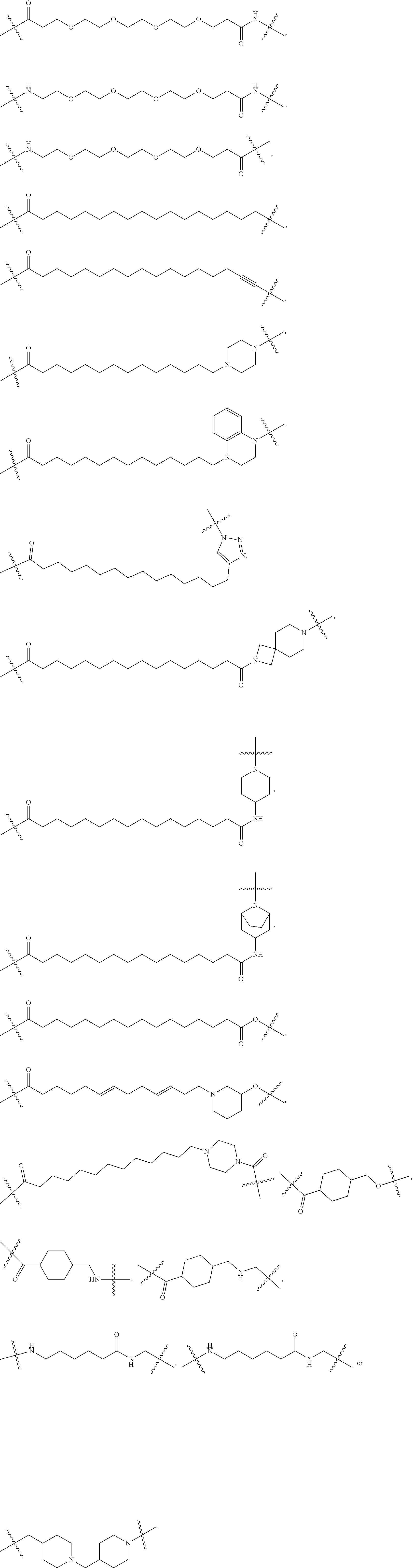

In some embodiments, PTM is selected from binding moieties of the following targeted proteins: ALK, AR, BET1, BRAF, BRCA2, BRD4, BRD9, BTK, BRM, CBL, CCNE1, CCNE2, CCR4, CCR7, CCR9, CD47, CLDN18, CYP, DDR1, DMPK, EGFR, ERBB2, ERBB3, ERBB4, FGFR1, FGFR2, FGFR3, FGFR4, GSPT1, JAK1, JAK3, KIF18A, KRAS, LCK, MET, NTRK1, NTRK2, NTRK3, PCSK9, PKMYT1, PARP7, PARP14, RAD51, RBM10, RET, RORA, STAT3, SOS1, TYK2, USP1, and USP14.

In some embodiments, PTM is selected from binding moieties of the following targeted proteins: ALK, AR, BET1, BRAF, BRCA2, BRD4, BRD9, BTK, CBL, CCNE1, CCNE2, CCR4, CCR7, CCR9, CD47, CLDN18, CYP, DDR1, DMPK, EGFR, ERBB2, ERBB3, ERBB4, FGFR1, FGFR2, FGFR3, FGFR4, GSPT1, JAK1, JAK3, KIF18A, KRAS, LCK, MET, NTRK1, NTRK2, NTRK3, PCSK9, PKMYT1, PARP7, PARP14, RAD51, RBM10, RET, RORA, STAT3, SOS1, TYK2, USP1, and USP14.

In some embodiments, PTM is selected from binding moieties of the following targeted proteins: BRM, BRD4, and STAT3.

In some embodiments, PTM is selected from binding moieties of the following targeted proteins: BRD4 and STAT3.

In some embodiments, PTM is selected from binding moieties of the following targeted proteins: BRM and BRD4.

In some embodiments, PTM is selected from the following structural groups:

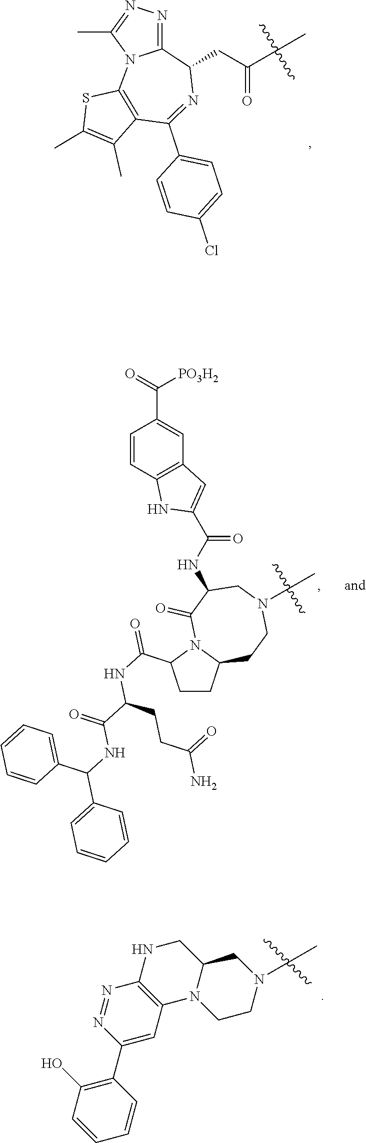

In some embodiments, the compound represented by formula (I) or the pharmaceutically acceptable salt thereof is selected from the following compounds or pharmaceutically acceptable salts thereof:

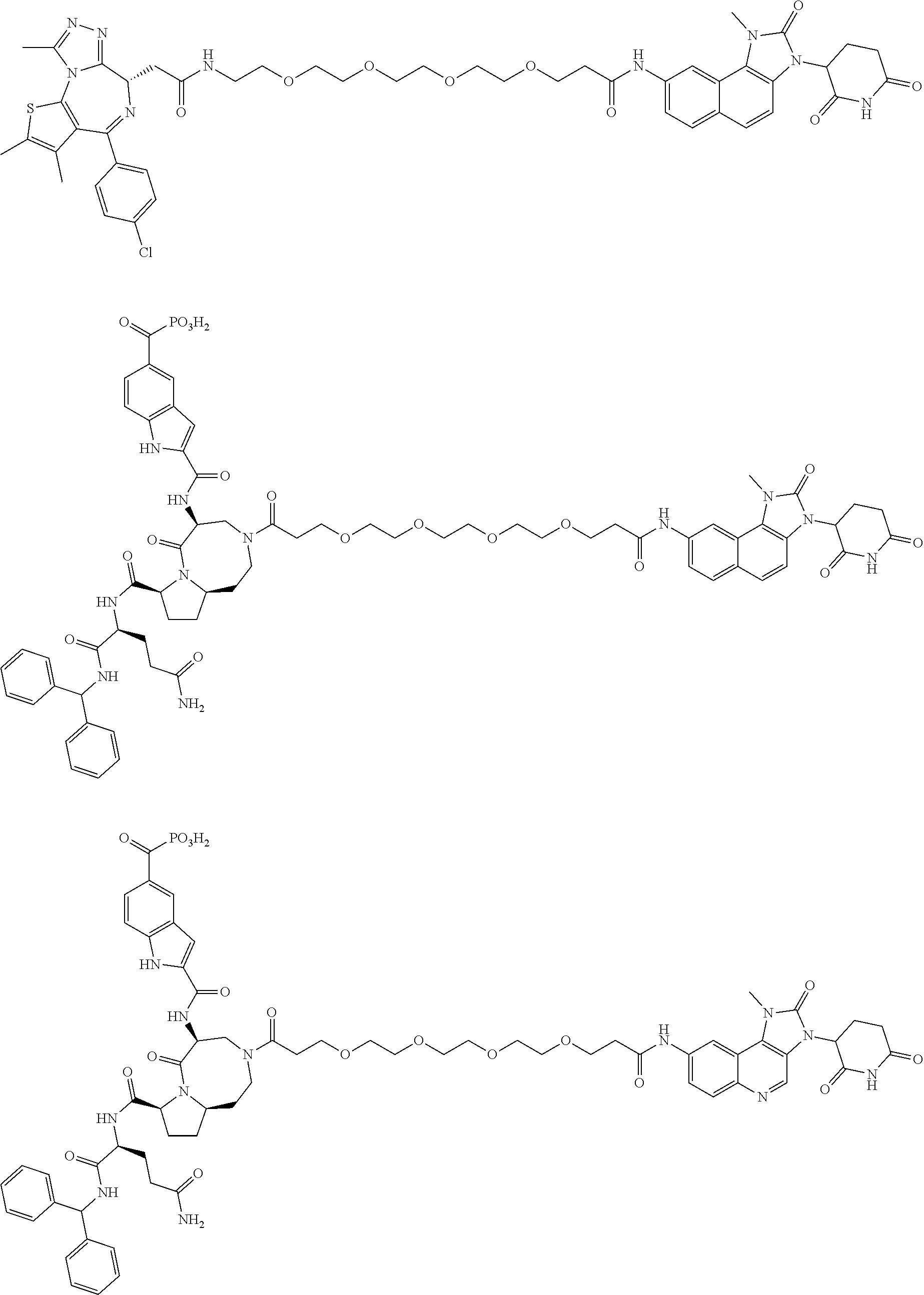

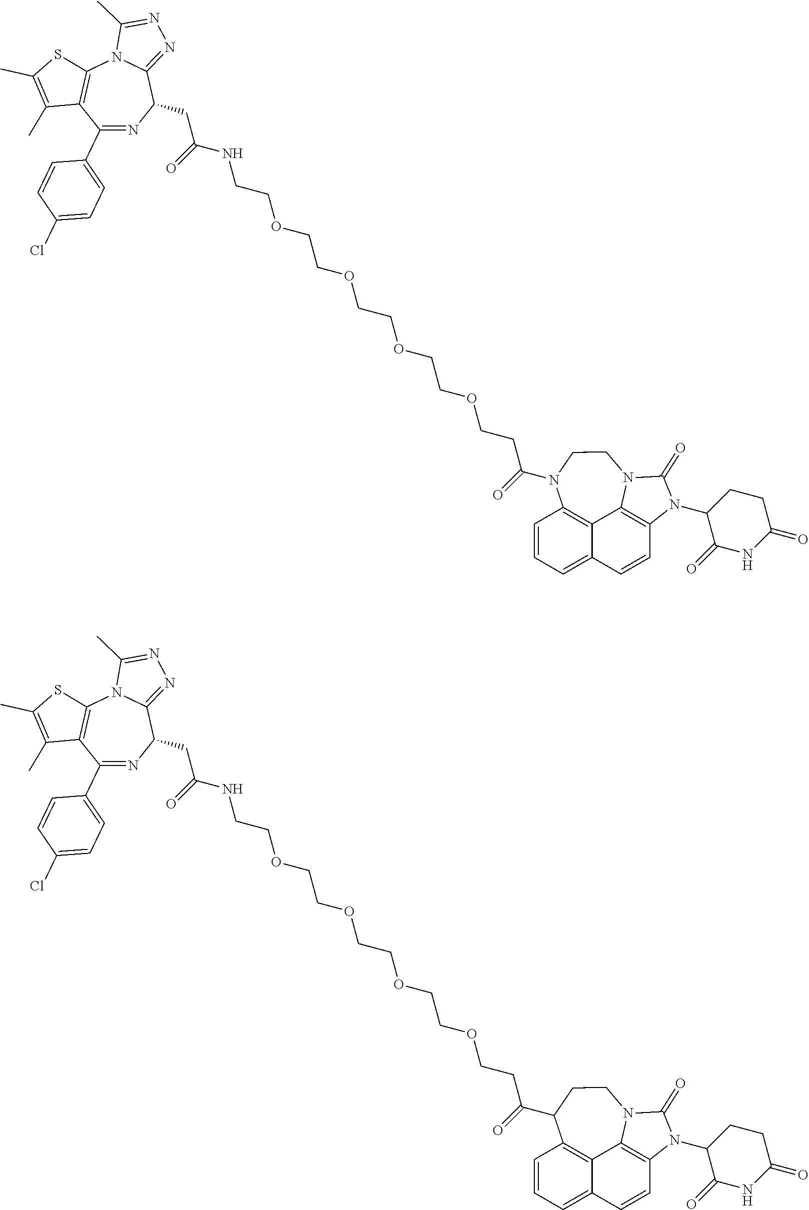 [SMILES: Cc1sc2c(c1C)C(c1ccc(Cl)cc1)=N[C@@H](CC(=O)NCCOCCOCCOCCOCCC(=O)N1CCn3c(=O)n(C4CCC(=O)NC4=O)c4ccc5cccc1c5c43)c1nnc(C)n1-2 ; Cc1sc2c(c1C)C(c1ccc(Cl)cc1)=N[C@@H](CC(=O)NCCOCCOCCOCCOCCC(=O)C1CCn3c(=O)n(C4CCC(=O)NC4=O)c4ccc5cccc1c5c43)c1nnc(C)n1-2]

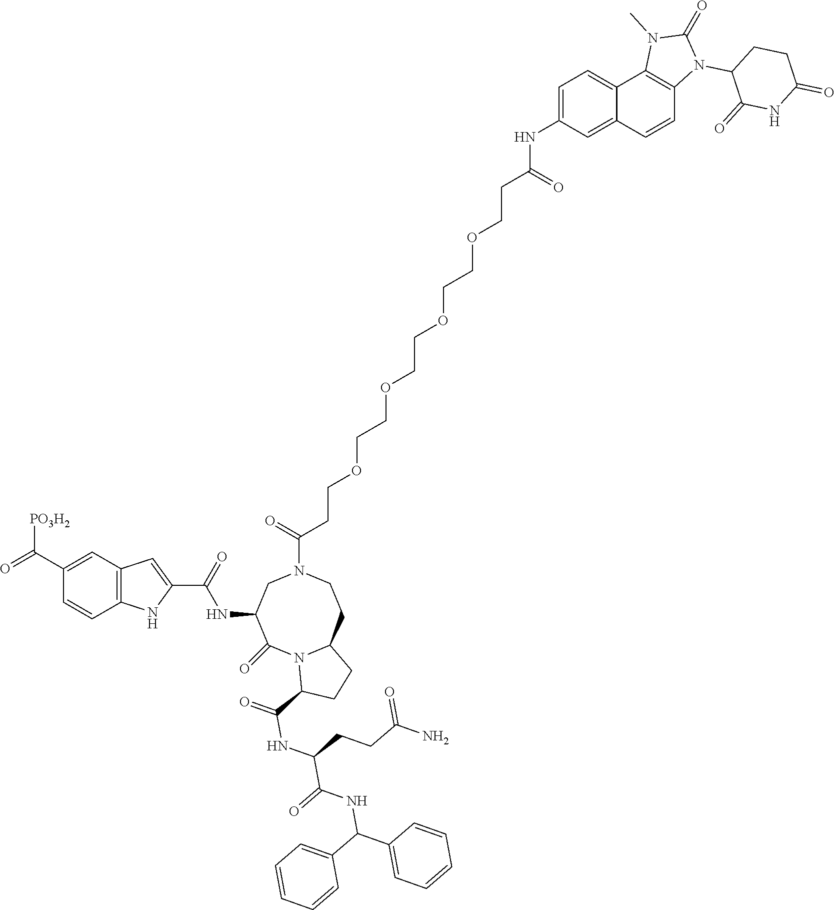

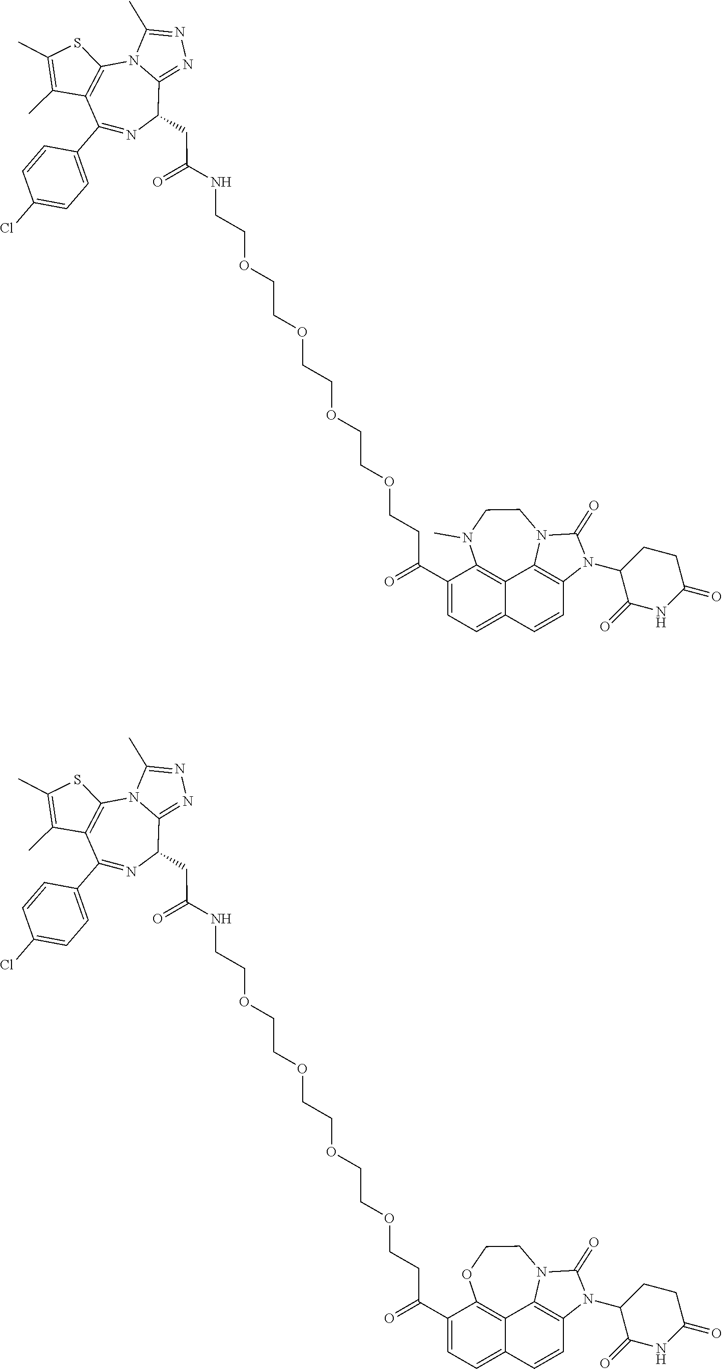 [SMILES: Cc1sc2c(c1C)C(c1ccc(Cl)cc1)=N[C@@H](CC(=O)NCCOCCOCCOCCOCCC(=O)c1ccc3ccc4c5c3c1N(C)CCn5c(=O)n4C1CCC(=O)NC1=O)c1nnc(C)n1-2 ; Cc1sc2c(c1C)C(c1ccc(Cl)cc1)=N[C@@H](CC(=O)NCCOCCOCCOCCOCCC(=O)c1ccc3ccc4c5c3c1OCCn5c(=O)n4C1CCC(=O)NC1=O)c1nnc(C)n1-2]

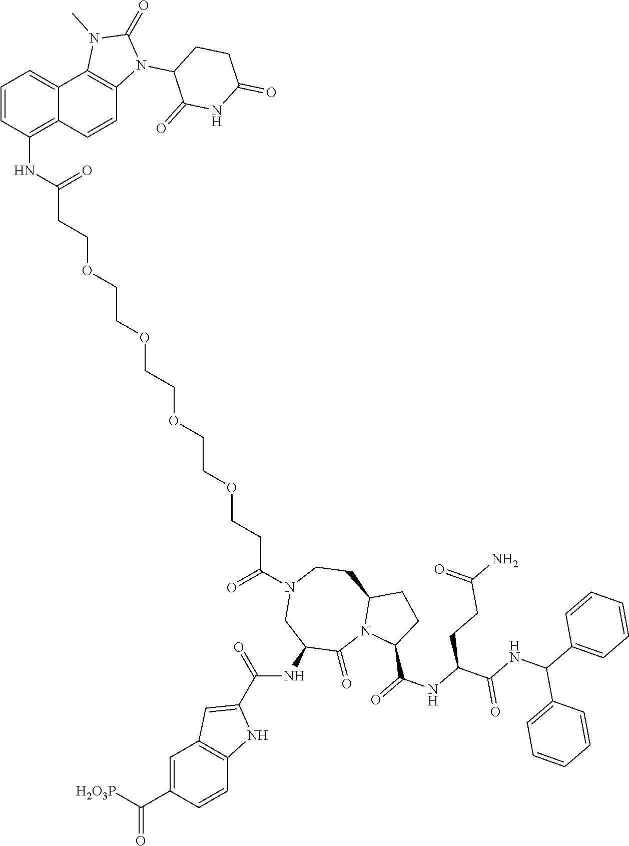

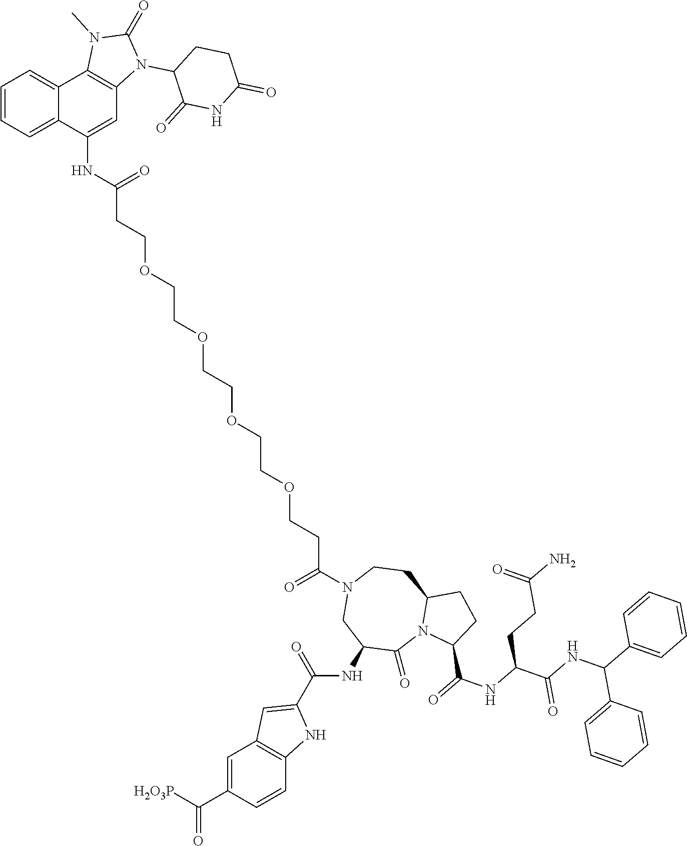

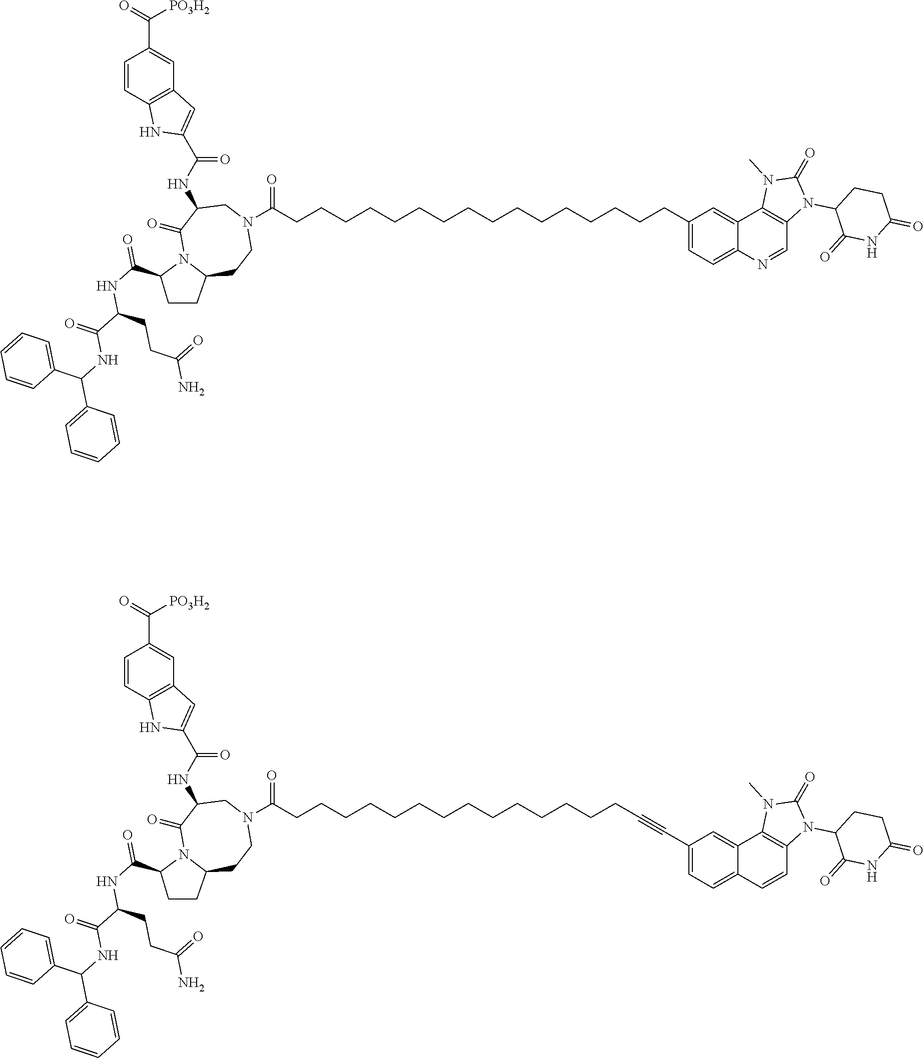

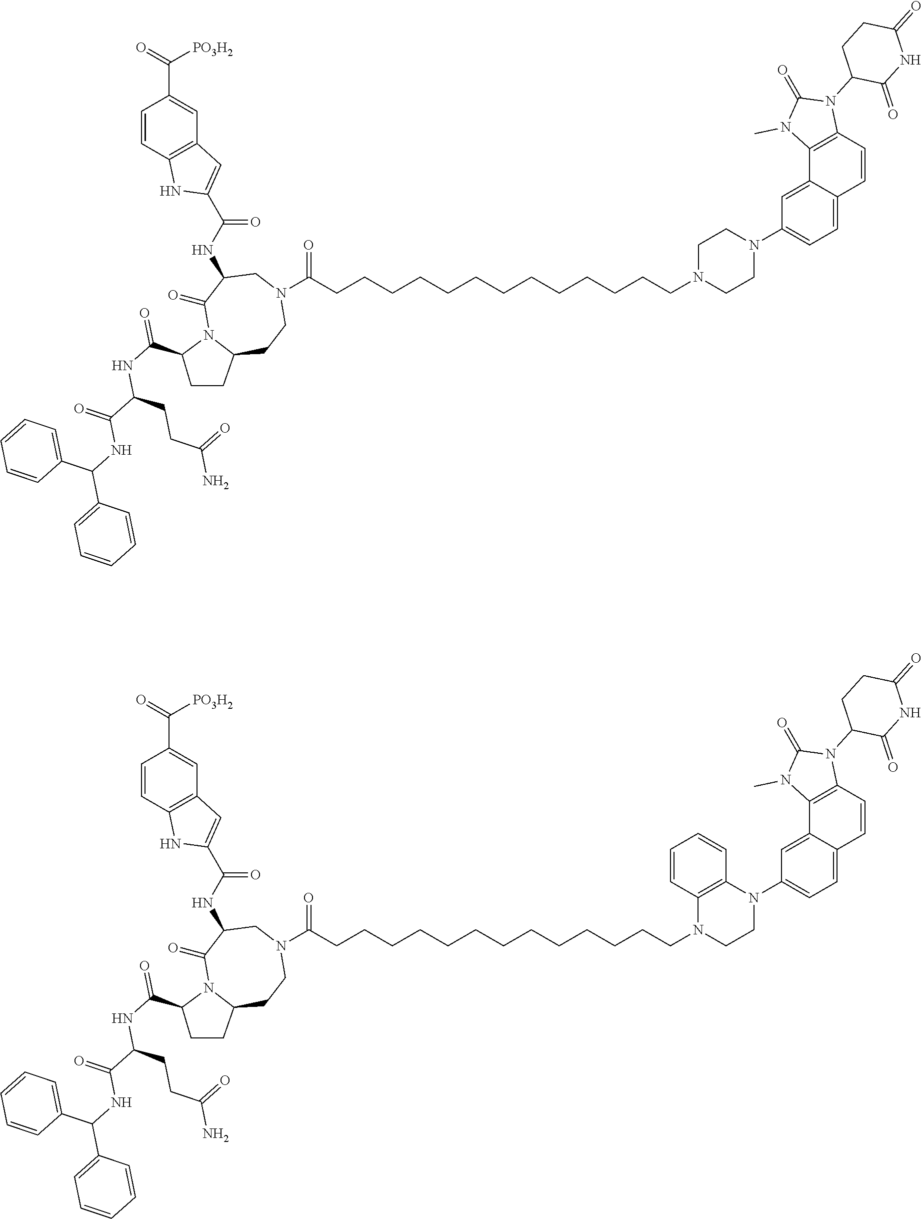

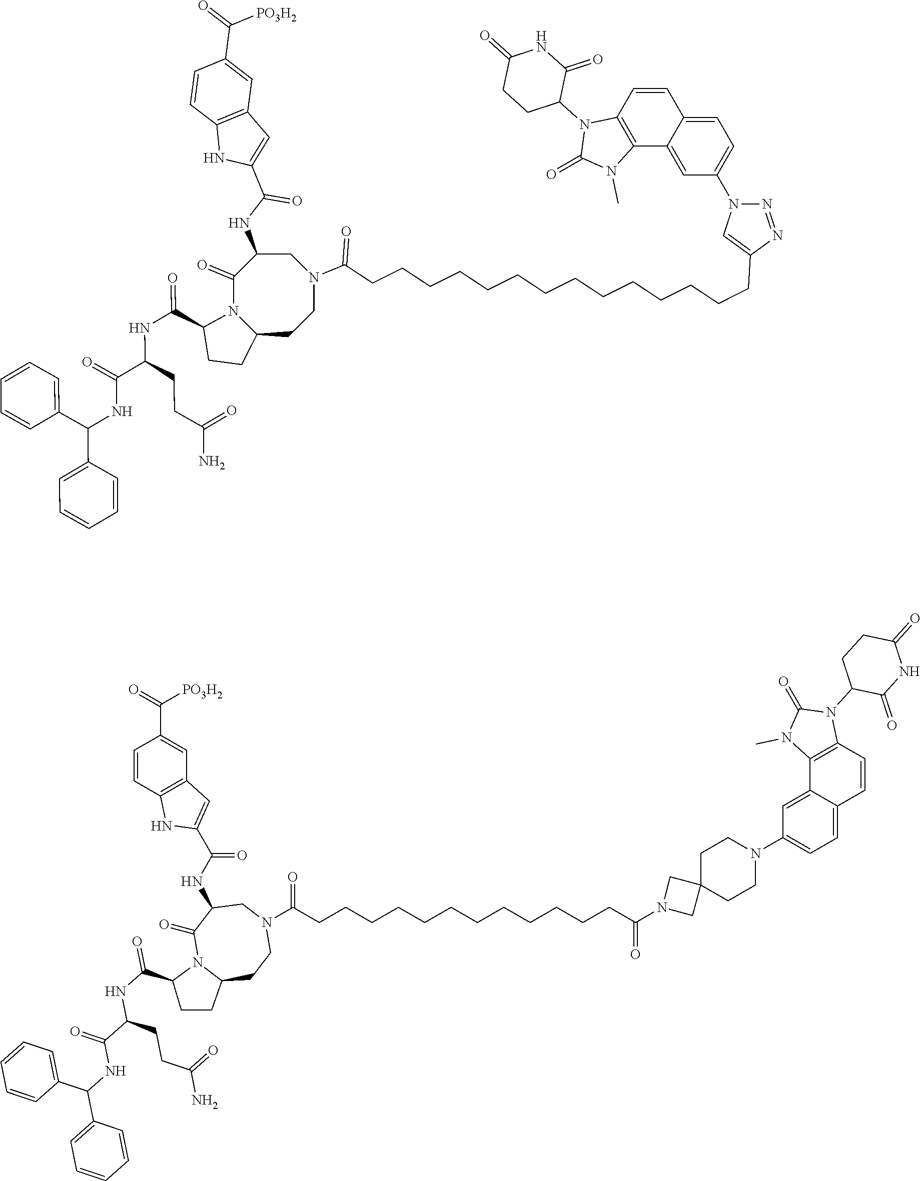

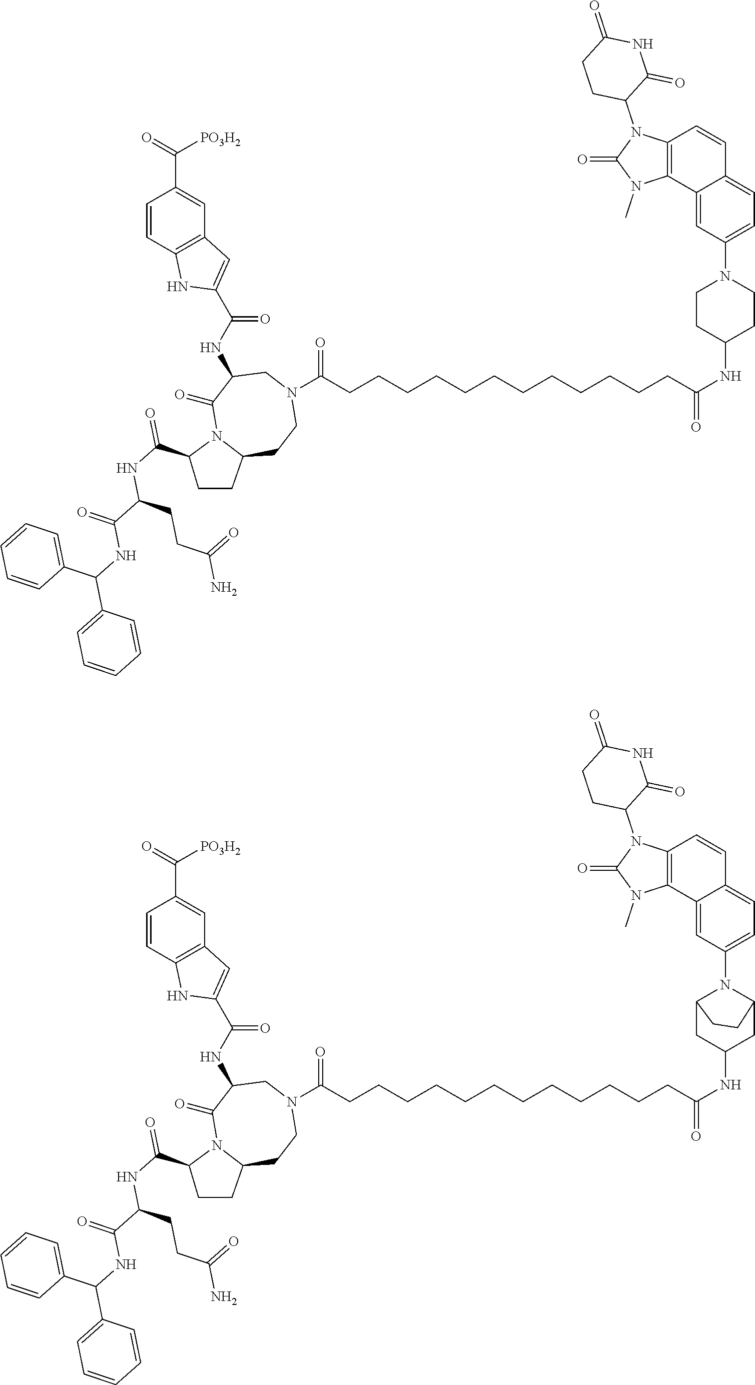

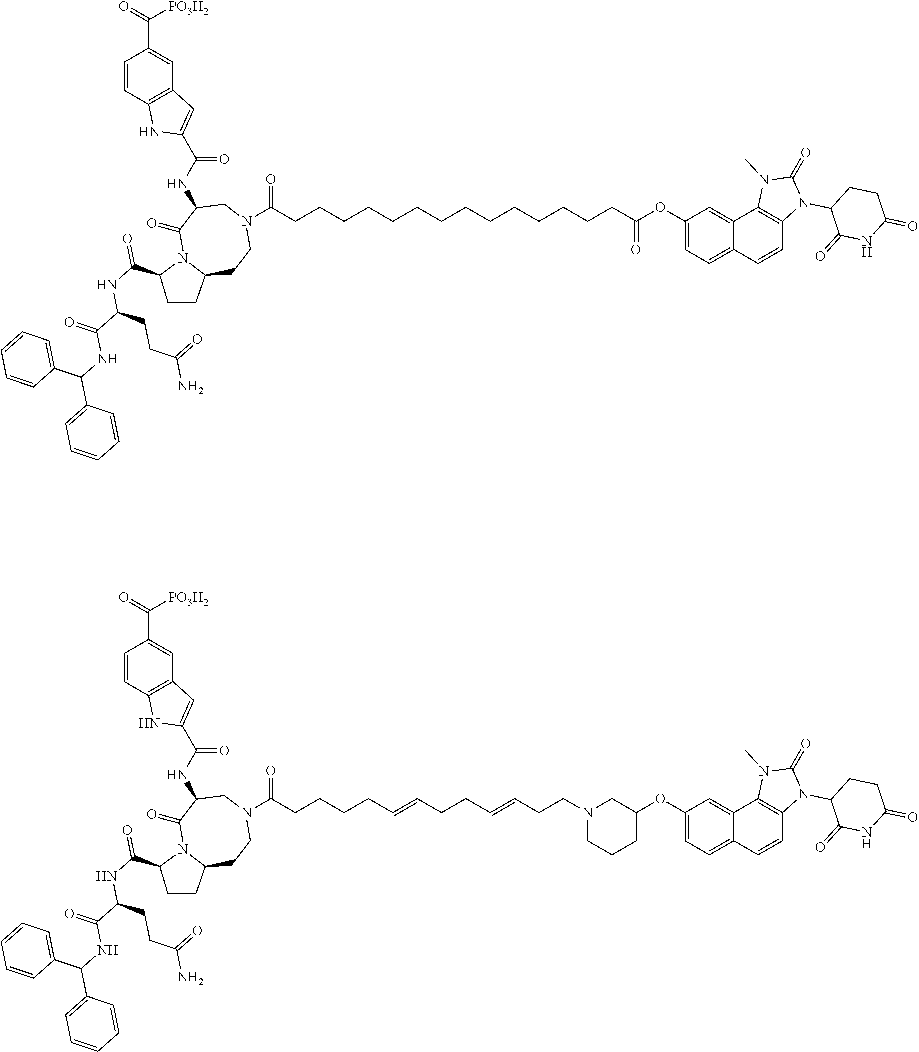

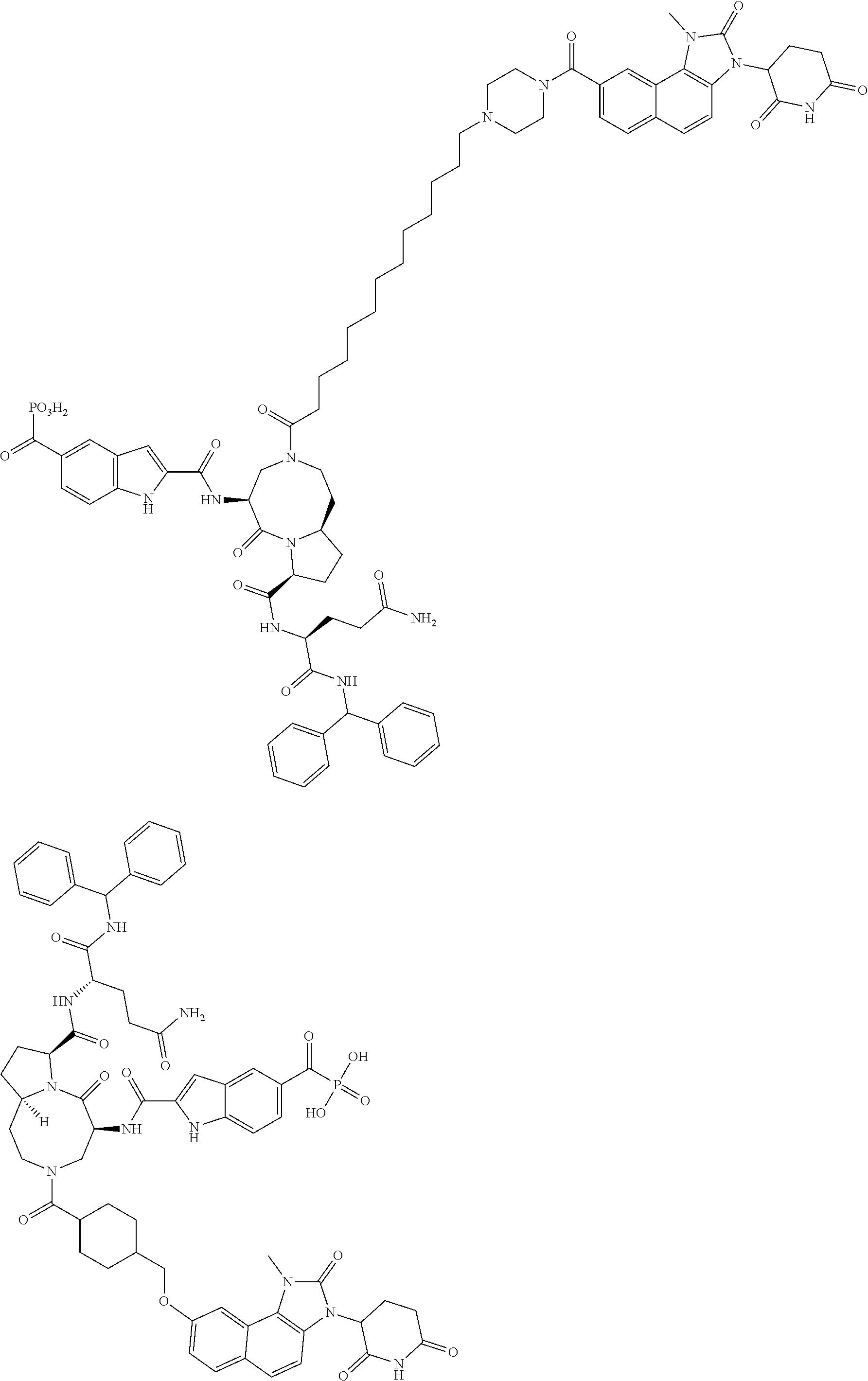

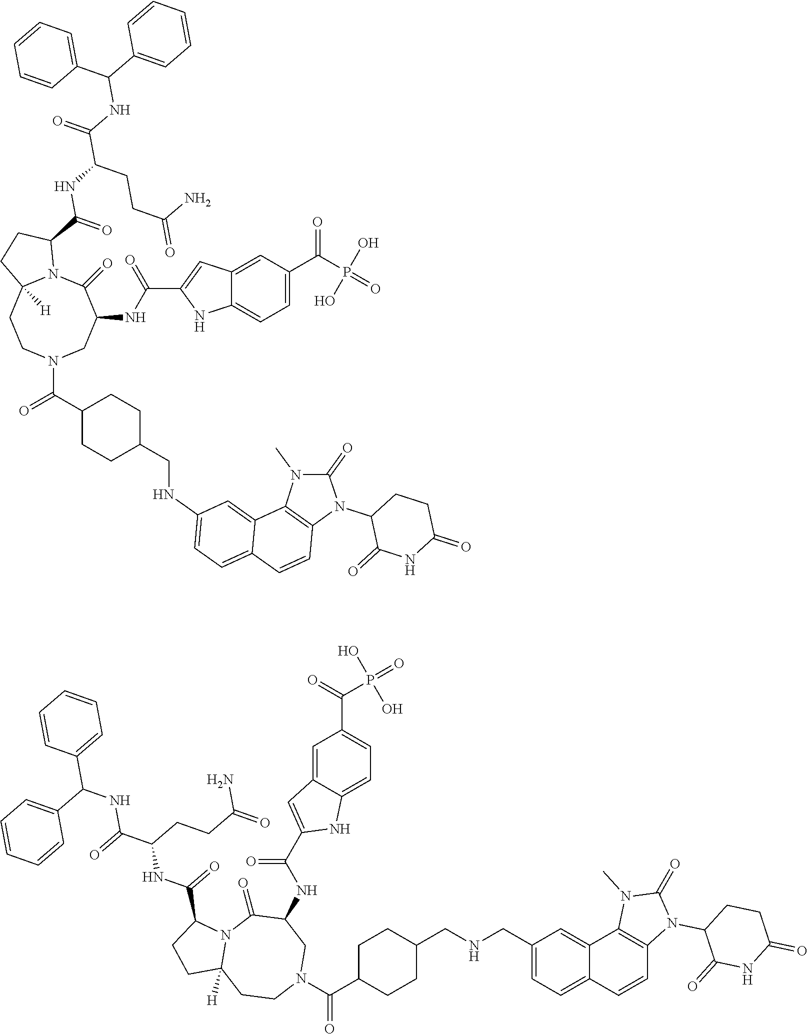

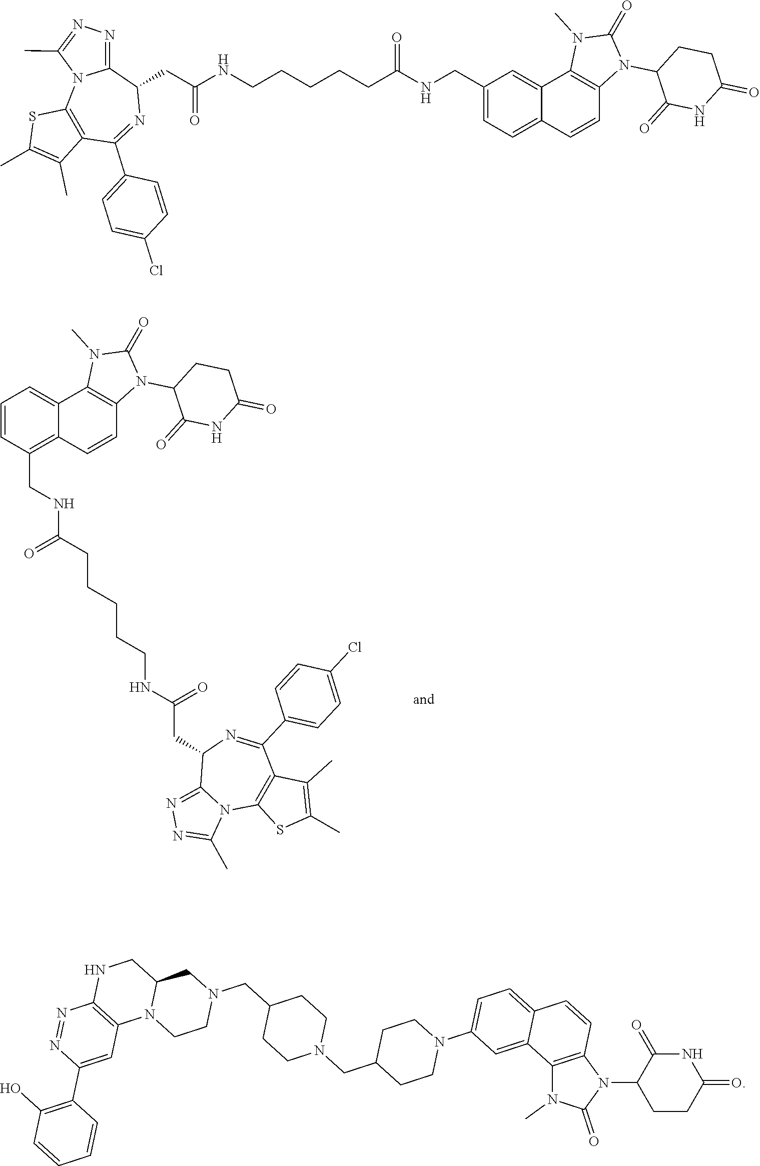 [SMILES: Cc1sc2c(c1C)C(c1ccc(Cl)cc1)=N[C@@H](CC(=O)NCCCCCC(=O)NCc1ccc3ccc4c(c3c1)n(C)c(=O)n4C1CCC(=O)NC1=O)c1nnc(C)n1-2 ; Cc1sc2c(c1C)C(c1ccc(Cl)cc1)=N[C@@H](CC(=O)NCCCCCC(=O)NCc1cccc3c1ccc1c3n(C)c(=O)n1C1CCC(=O)NC1=O)c1nnc(C)n1-2 ; Cn1c(=O)n(C2CCC(=O)NC2=O)c2ccc3ccc(N4CCC(CN5CCC(CN6CCN7c8cc(-c9ccccc9O)nnc8NC[C@H]7C6)CC5)CC4)cc3c21]

In another aspect, the present disclosure provides a pharmaceutical composition comprising the compound represented by formula (I) or the pharmaceutically acceptable salt thereof of the present disclosure and a pharmaceutically acceptable excipient.

In another aspect, the present disclosure provides a method for treating an abnormal cell proliferation disease in a mammal, which comprises administering to a mammal, preferably a human, in need of the treatment a therapeutically effective amount of the compound represented by formula (I) or the pharmaceutically acceptable salt thereof, or the pharmaceutical composition thereof.

In another aspect, the present disclosure provides use of the compound represented by formula (I) or the pharmaceutically acceptable salt thereof, or the pharmaceutical composition thereof in the preparation of a medicament for preventing or treating an abnormal cell proliferation disease.

In another aspect, the present disclosure provides use of the compound represented by formula (I) or the pharmaceutically acceptable salt thereof, or the pharmaceutical composition thereof in the prevention or treatment of an abnormal cell proliferation disease.

In another aspect, the present disclosure provides the compound represented by formula (I) or the pharmaceutically acceptable salt thereof, or the pharmaceutical composition thereof for use in preventing or treating an abnormal cell proliferation disease.

In some embodiments, the abnormal cell proliferation disease is cancer.

In some embodiments, the cancer is selected from a solid tumor, an adenocarcinoma, and a hematologic cancer.

In another aspect, the present disclosure also provides a compound represented by formula (III) or a pharmaceutically acceptable salt thereof:

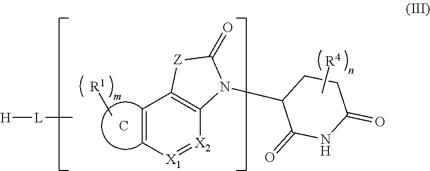

wherein X₁ and X₂ are independently selected from N and CH, wherein the CH is optionally substituted with R²; Z, R¹, R², R⁴, m, n, and L are as defined above;
ring C is selected from 5- to 6-membered heteroaromatic ring and benzene ring;
provided that the following compounds or pharmaceutically acceptable salts thereof are excluded:

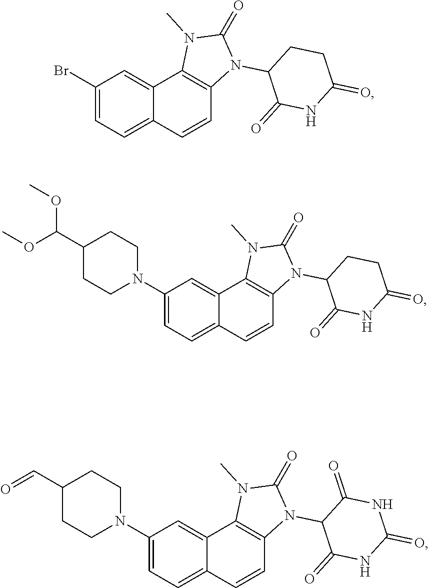 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

In some embodiments, the compound represented by formula (III) or the pharmaceutically acceptable salt thereof is selected from a compound represented by formula (III-1a) or formula (III-1b) or a pharmaceutically acceptable salt thereof:

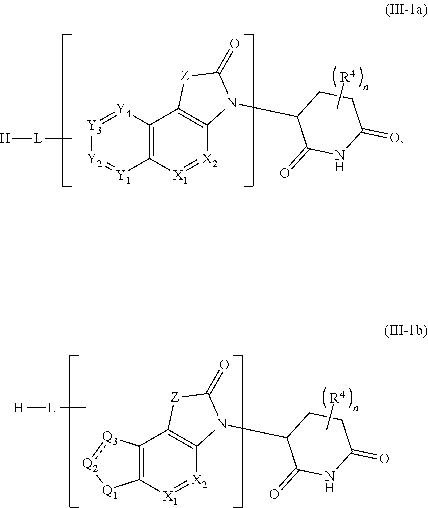

wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a double bond; Q₁, Q₂, and Q₃ are independently selected from O, S, NH, CH₂, N, and CH, wherein the NH, CH₂, or CH is optionally substituted with R¹; Z, R¹, R², R⁴, n, and L are as defined above;
provided that the following compounds or pharmaceutically acceptable salts thereof are excluded:

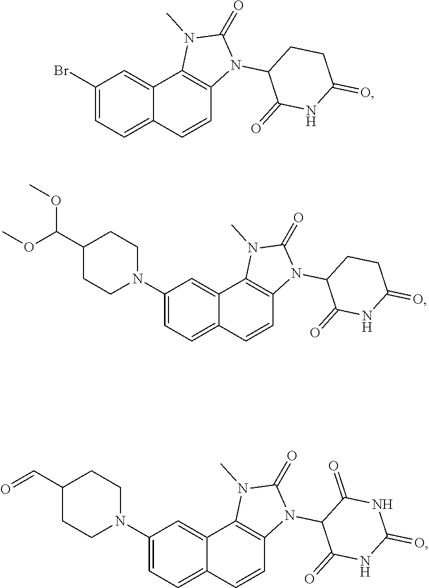 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

In some embodiments, the compound represented by formula (III) or the pharmaceutically acceptable salt thereof is selected from the following compounds or pharmaceutically acceptable salts thereof:

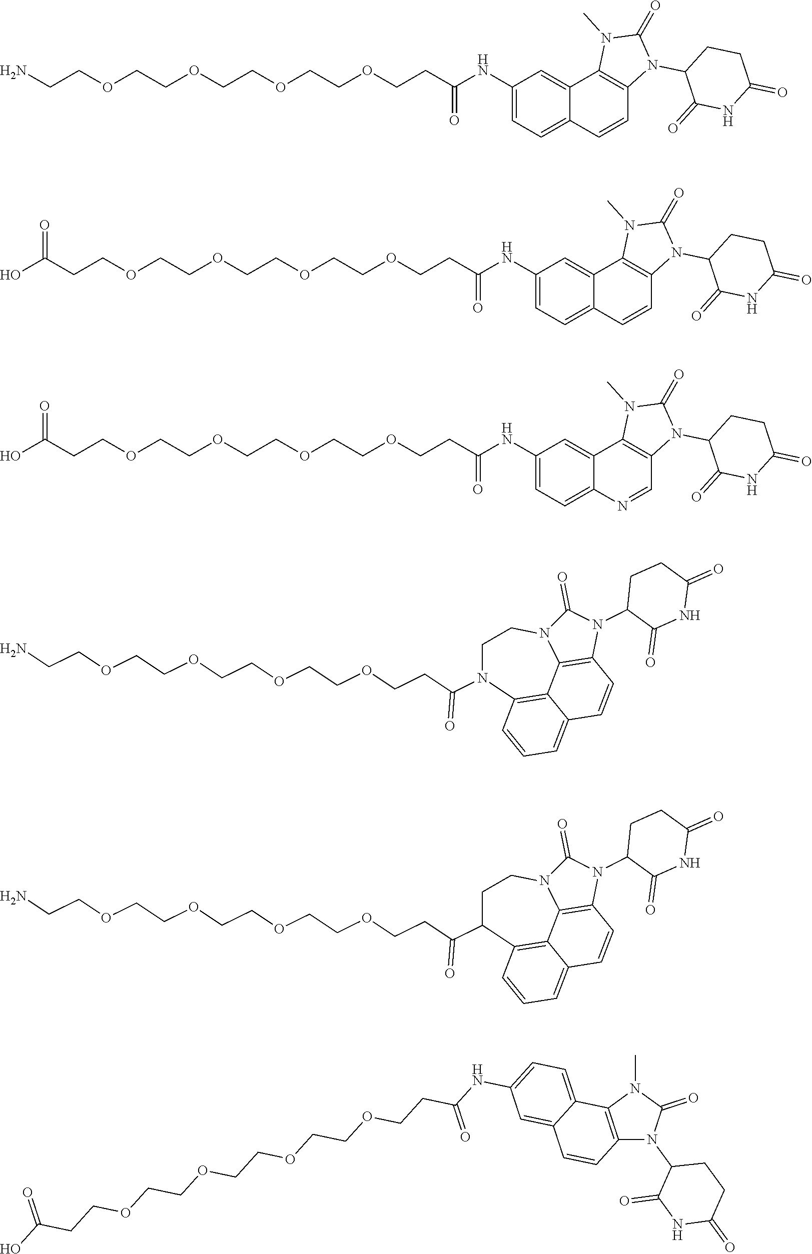 [SMILES: Cn1c(=O)n(C2CCC(=O)NC2=O)c2ccc3ccc(NC(=O)CCOCCOCCOCCOCCN)cc3c21 ; Cn1c(=O)n(C2CCC(=O)NC2=O)c2ccc3ccc(NC(=O)CCOCCOCCOCCOCCC(=O)O)cc3c21 ; Cn1c(=O)n(C2CCC(=O)NC2=O)c2cnc3ccc(NC(=O)CCOCCOCCOCCOCCC(=O)O)cc3c21 ; NCCOCCOCCOCCOCCC(=O)N1CCn2c(=O)n(C3CCC(=O)NC3=O)c3ccc4cccc1c4c32 ; NCCOCCOCCOCCOCCC(=O)C1CCn2c(=O)n(C3CCC(=O)NC3=O)c3ccc4cccc1c4c32 ; Cn1c(=O)n(C2CCC(=O)NC2=O)c2ccc3cc(NC(=O)CCOCCOCCOCCOCCC(=O)O)ccc3c21]

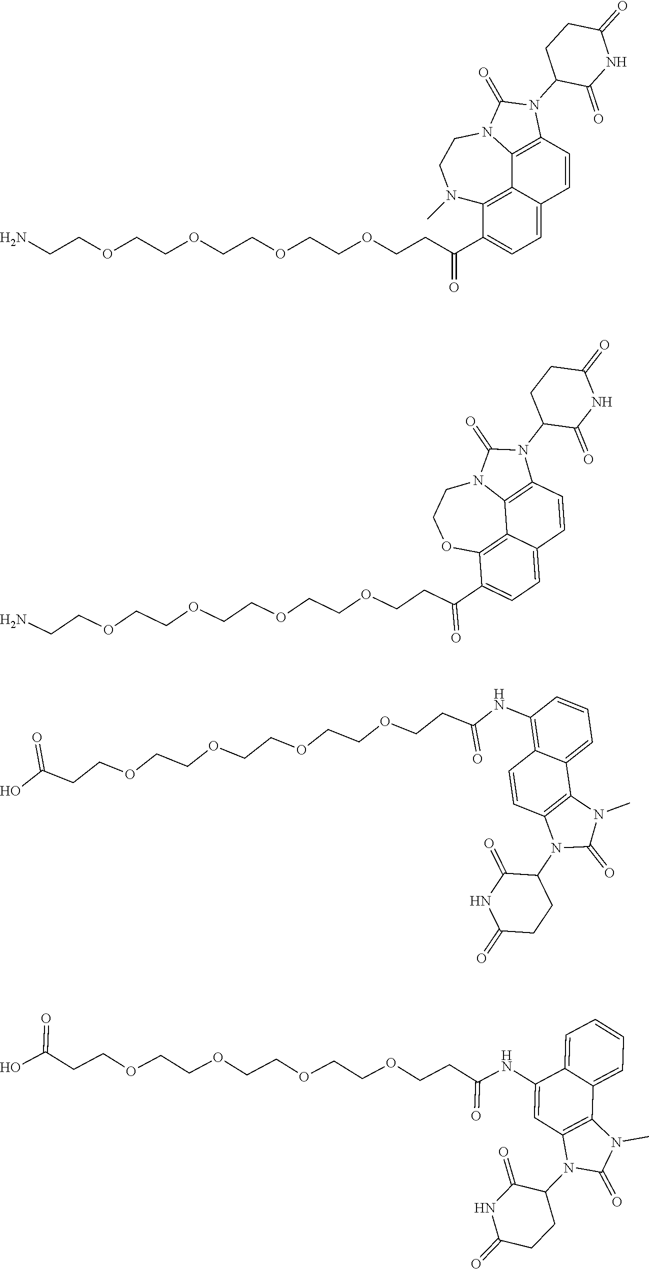 [SMILES: CN1CCn2c(=O)n(C3CCC(=O)NC3=O)c3ccc4ccc(C(=O)CCOCCOCCOCCOCCN)c1c4c32 ; NCCOCCOCCOCCOCCC(=O)c1ccc2ccc3c4c2c1OCCn4c(=O)n3C1CCC(=O)NC1=O ; Cn1c(=O)n(C2CCC(=O)NC2=O)c2ccc3c(NC(=O)CCOCCOCCOCCOCCC(=O)O)cccc3c21 ; Cn1c(=O)n(C2CCC(=O)NC2=O)c2cc(NC(=O)CCOCCOCCOCCOCCC(=O)O)c3ccccc3c21]

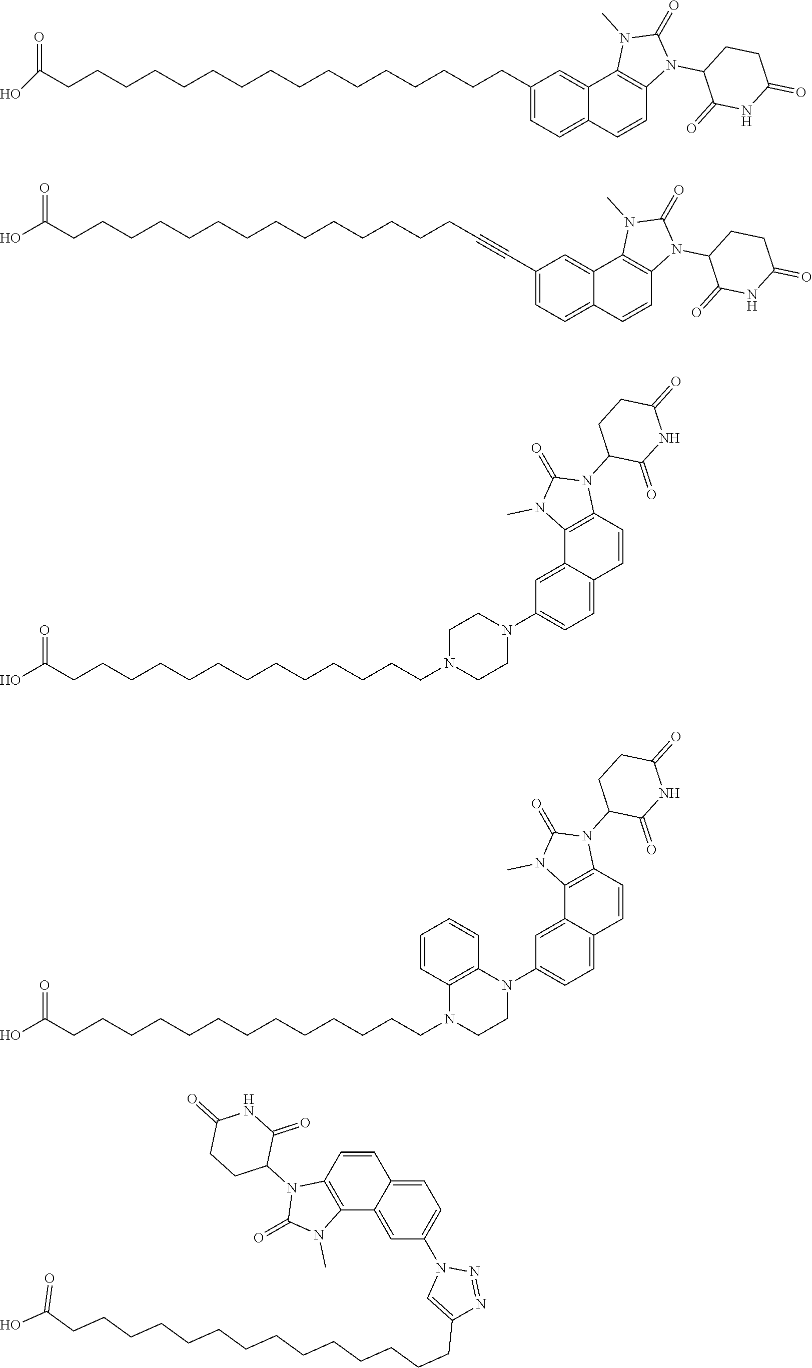

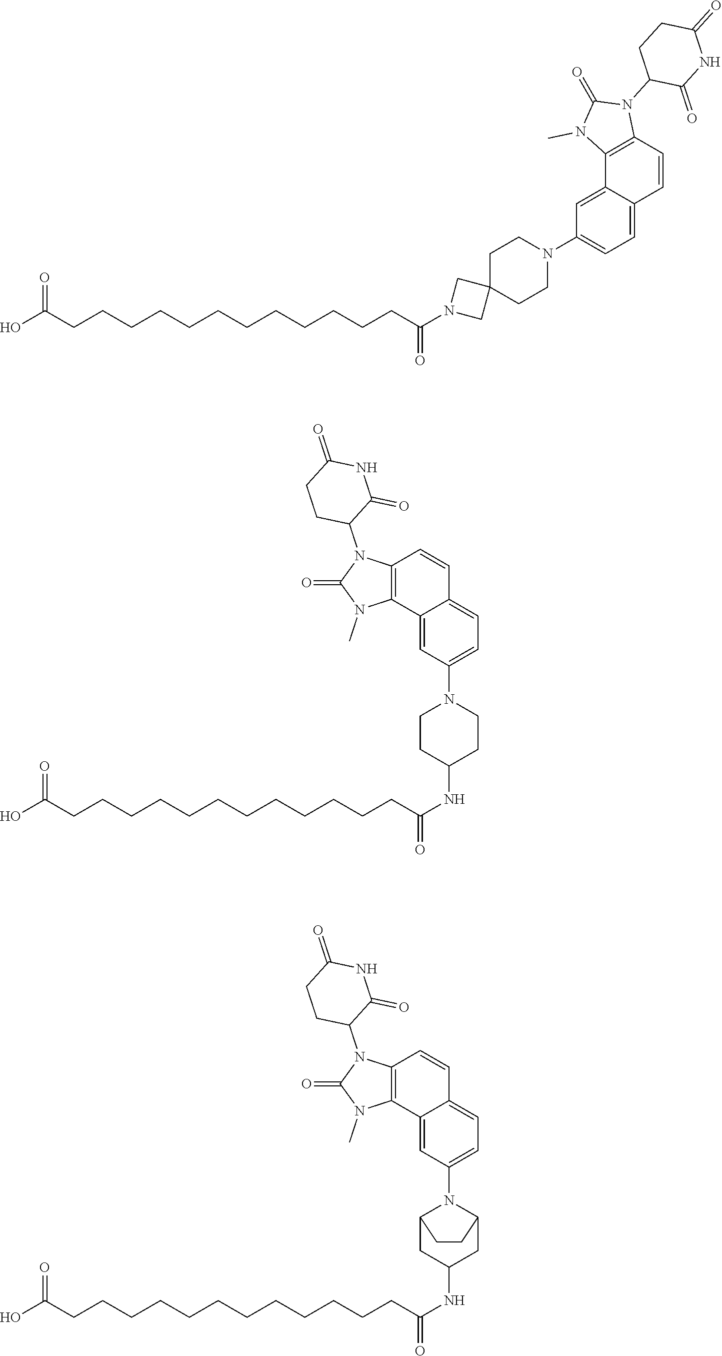 [SMILES: Cn1c(=O)n(C2CCC(=O)NC2=O)c2ccc3ccc(N4CCC5(CC4)CN(C(=O)CCCCCCCCCCCCC(=O)O)C5)cc3c21 ; Cn1c(=O)n(C2CCC(=O)NC2=O)c2ccc3ccc(N4CCC(NC(=O)CCCCCCCCCCCCC(=O)O)CC4)cc3c21 ; Cn1c(=O)n(C2CCC(=O)NC2=O)c2ccc3ccc(N4C5CCC4CC(NC(=O)CCCCCCCCCCCCC(=O)O)C5)cc3c21]

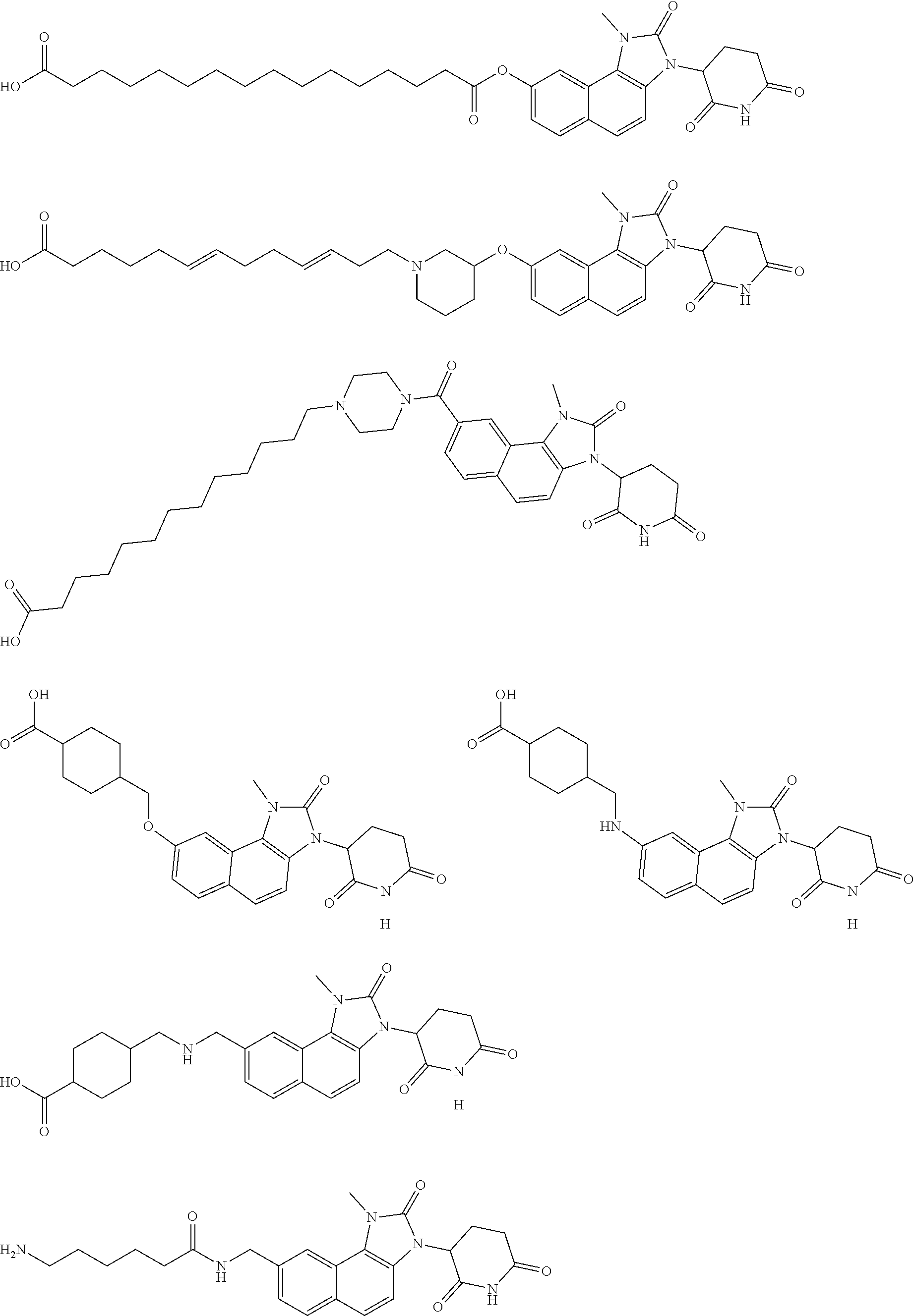

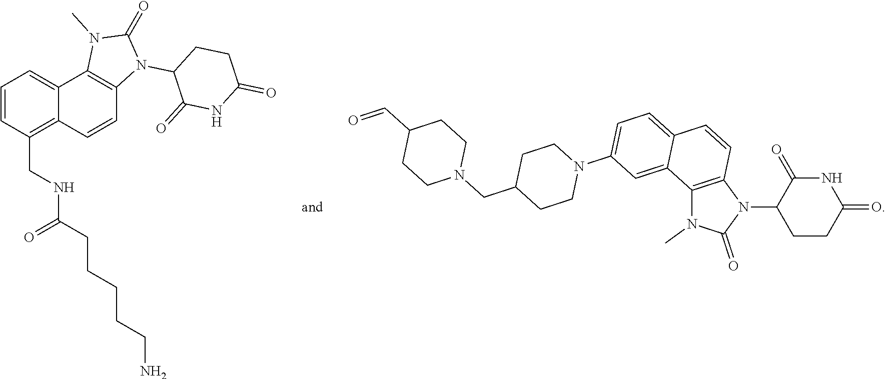 [SMILES: Cn1c(=O)n(C2CCC(=O)NC2=O)c2ccc3c(CNC(=O)CCCCCN)cccc3c21 ; Cn1c(=O)n(C2CCC(=O)NC2=O)c2ccc3ccc(N4CCC(CN5CCC(C=O)CC5)CC4)cc3c21]

In another aspect, the present disclosure also provides a compound represented by formula (IV) or a pharmaceutically acceptable salt thereof:

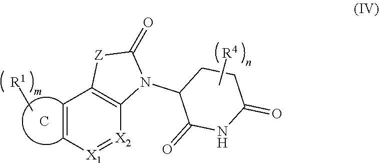

wherein X₁ and X₂ are independently selected from N and CH, wherein the CH is optionally substituted with R²; Z, R¹, R², R⁴, m, and n are as defined above;
ring C is selected from 5- to 6-membered heteroaromatic ring and benzene ring;
provided that the following compounds or pharmaceutically acceptable salts thereof are excluded:

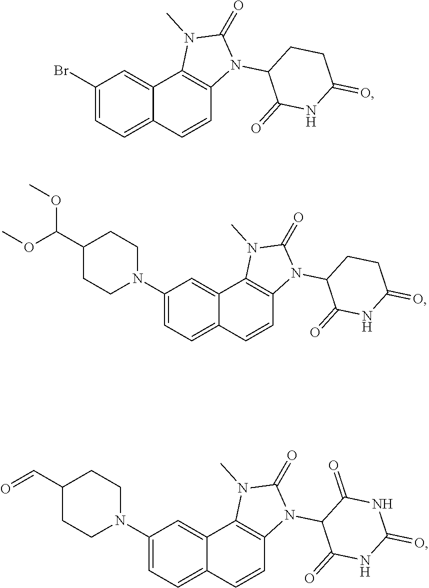 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

In some embodiments, the compound represented by formula (IV) or the pharmaceutically acceptable salt thereof is selected from a compound represented by formula (IV-1a) or formula (IV-1b) or a pharmaceutically acceptable salt thereof:

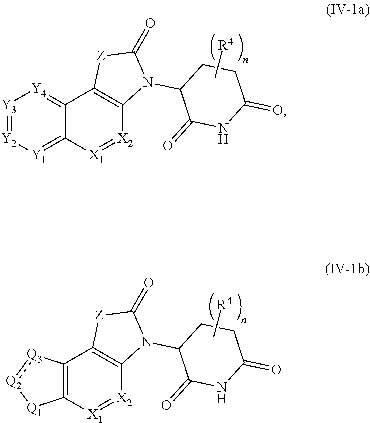

wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a double bond; Q₁, Q₂, and Q₃ are independently selected from O, S, NH, CH₂, N, and CH, wherein the NH, CH₂, or CH is optionally substituted with R¹; Z, R¹, R², R⁴, and n are as defined above;
provided that the following compounds or pharmaceutically acceptable salts thereof are excluded:

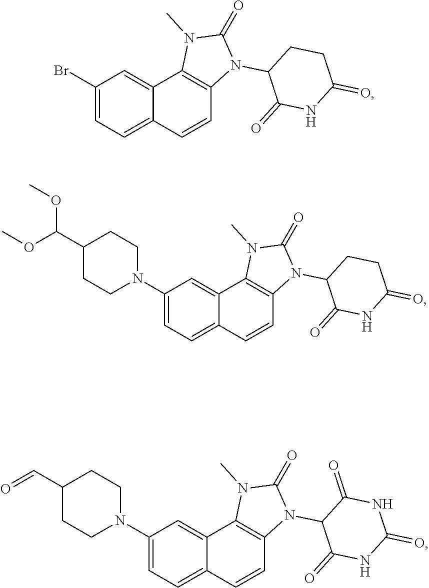 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

In another aspect, the present disclosure also provides a compound represented by formula (V) or a pharmaceutically acceptable salt thereof,

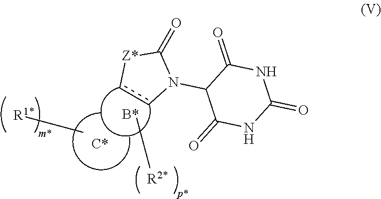

wherein
Z* is selected from C(R^{3*})₂, NR^{3*}, and O;
ring B* is selected from 5- to 6-membered heteroaromatic ring, 5- to 8-membered heterocyclic ring, benzene ring, and C₅-C₈ saturated or partially saturated carbon ring;
ring C* is selected from 5- to 6-membered heteroaromatic ring, 5- to 8-membered heterocyclic ring, benzene ring, and C₅-C₈ saturated or partially saturated carbon ring;
R^{1*}, R^{2*}, R^{3*}, and R^{5*} are each independently selected from the following groups:
(a) halogen, ═O, CN, NO₂, —OR^{b*}, —N(R^{b*})₂, —S(O)R^{b*}, —SO₂R^{b*}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a*};
or

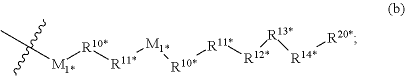

M_{1*} is selected from bond, —NR^{b*}—, —C(O)—, —C(O)O—, —SO₂—, —S(O)—, —O—, —S—, —C(O)NR^{b*}—, —C(═NR^{b*})—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroaryl, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R^{a*};
R^{10*}, R^{11*}, R^{12*}, R^{13*} and R^{14*} are independently selected from bond, —NR^{b*}—, —C(O)—, —C(O)O—, —SO₂—, —S(O)—, —O—, —S—, —NR^{b*}C(O)—, —C(═NR^{b*})—, —C(S)—, —P(O)(OR^{b*})O—, —P(O)(OR^{b*})—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R^{a*};
R^{20*} is selected from H, halogen, CN, —OR^{b*}, —N(R^{b*})₂, —S(O)R^{b*}, —SO₂R^{b*}, —C(O)R^{b*}, —C(O)OR^{b*}, —OC(O)R^{b*}, —C(O)N(R^{b*})₂, —NR^{b*}C(O)R^{b*}, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a*};
or R^{1*} and R^{3*}, together with the atoms linked thereto, form C₅-C₈ saturated or partially saturated carbon ring, 5- to 6-membered heteroaromatic ring, or 5- to 8-membered heterocyclic ring, wherein the C₅-C₈ saturated or partially saturated carbon ring, 5- to 6-membered heteroaromatic ring, or 5- to 8-membered heterocyclic ring is optionally substituted with R^{5*};
each R^{4*} is selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a*};
each R^{a*} is independently selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c*};
each R^{b*} is independently selected from H, halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c*};
each R^{c*} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d*};
each R^{d*} is independently selected from halogen, CN, OH, NH₂, and C₁-C₆ alkyl;
n* is independently selected from 0, 1, 2, 3, and 4;
m* and p* are independently selected from 0, 1, 2, 3, 4, 5, and 6.

In some embodiments, Z* is selected from NR^{3*} and O.

In some embodiments, ring B* is selected from 5- to 6-membered heteroaromatic ring, 5- to 6-membered heterocyclic ring, a benzene ring, and C₅-C₆ saturated or partially saturated carbon ring.

In some embodiments, ring B* is selected from 5- to 6-membered heteroaromatic ring and benzene ring.

In some embodiments, ring B* is selected from pyridine ring and benzene ring.

In some embodiments, ring C* is selected from 5- to 6-membered heteroaromatic ring, 5- to 6-membered heterocyclic ring, a benzene ring, and C₅-C₆ saturated or partially saturated carbon ring.

In some embodiments, ring C* is selected from 5- to 6-membered heteroaromatic ring and benzene ring.

In some embodiments, ring C* is selected from benzene ring, pyridine ring, pyrrole ring, and thiazole ring.

In some embodiments, R^{1*} and R^{2*} are independently selected from halogen, CN, NO₂, —OR^{b*}, —N(R^{b*})₂, —S(O)R^{b*}, —SO₂R^{b*}, C₁-C₁₀ alkyl, and C₃-C₁₀ cycloalkyl, wherein the C₁-C₁₀ alkyl or C₃-C₁₀ cycloalkyl is optionally substituted with R^{a*}.

In some embodiments, R^{1*} and R^{2*} are independently selected from halogen, CN, OH, NH₂, 2- to 10-membered heteroalkyl, C₁-C₁₀ alkyl, and C₃-C₁₀ cycloalkyl, wherein the 2- to 10-membered heteroalkyl, C₁-C₁₀ alkyl, or C₃-C₁₀ cycloalkyl is optionally substituted with R^{a*}.

In some embodiments, R^{3*} is selected from halogen, CN, OH, NH₂, 2- to 10-membered heteroalkyl, C₁-C₁₀ alkyl, and C₃-C₁₀ cycloalkyl, wherein the 2- to 10-membered heteroalkyl, C₁-C₁₀ alkyl, or C₃-C₁₀ cycloalkyl is optionally substituted with R^{a*}.

In some embodiments, R^{3*} is selected from 2- to 6-membered heteroalkyl, C₁-C₆ alkyl, and C₃-C₆ cycloalkyl, wherein the 2- to 6-membered heteroalkyl, C₁-C₆ alkyl, or C₃-C₆ cycloalkyl is optionally substituted with R^{a*}.

In some embodiments, R^{3*} is selected from C₁-C₆ alkyl and C₃-C₆ cycloalkyl, wherein the C₁-C₆ alkyl or C₃-C₆ cycloalkyl is optionally substituted with R^{a*}.

In some embodiments, R^{3*} is independently selected from CH₃, CH₂CH₃, CH(CH₃)₂, CHCF₃, and cyclopropyl.

In some embodiments, R^{5*} is selected from halogen, ═O, CN, OH, NH₂, C₁-C₁₀ alkyl, and C₃-C₁₀ cycloalkyl, wherein the C₁-C₁₀ alkyl or C₃-C₁₀ cycloalkyl is optionally substituted with R^{a*}.

In some embodiments, R^{5*} is selected from halogen, ═O, CN, OH, NH₂, C₁-C₆ alkyl, and C₃-C₆ cycloalkyl, wherein the C₁-C₆ alkyl or C₃-C₆ cycloalkyl is optionally substituted with R^{a*}.

In some embodiments, R^{5*} is selected from ═O and C₁-C₆ alkyl, wherein the C₁-C₆ alkyl is optionally substituted with R^{a*}.

In some embodiments, R^{1*} is selected from

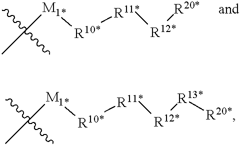

wherein M_{1*}, R^{10*}, R^{11*}, R^{12*}, R^{13*}, and R^{20*} are as defined above.

In some embodiments, R^{1*} is selected from

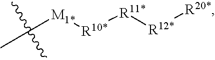

wherein M_{1*}, R^{10*}, R^{11*}, R^{12*}, and R^{20*} are as defined above.

In some embodiments, R^{2*} is selected from

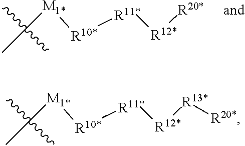

wherein M_{1*}, R^{10*}, R^{11*}, R^{12*}, R^{13*}, and R^{20*} are as defined above.

In some embodiments, R^{2*} is selected from

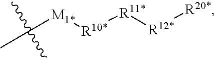

wherein M_{1*}, R^{10*}, R^{11*}, R^{12*}, and R^{20*} are as defined above.

In some embodiments, R^{3*} is selected from

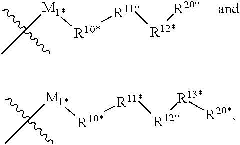

wherein M_{1*}, R^{10*}, R^{11*}, R^{12*}, R^{13*}, and R^{20*} are as defined above.

In some embodiments, R^{3*} is selected from

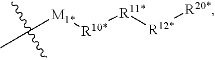

wherein M_{1*}, R^{10*}, R^{11*}, R^{12*}, and R^{20*} are as defined above.

In some embodiments, R^{5*} is selected from

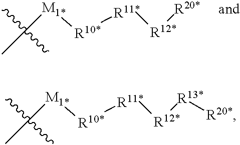

wherein M_{1*}, R^{10*}, R^{11*}, R^{12*}, R^{13*}, and R^{20*} are as defined above.

In some embodiments, R^{5*} is selected from

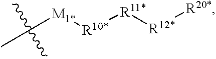

wherein M_{1*}, R^{10*}, R^{11*}, R^{12*}, and R^{20*} are as defined above.

In some embodiments, R^{1*}, R^{2*}, R^{3*}, and R^{5*} are independently selected from

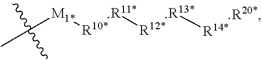

wherein R^{11*}, R^{12*}, and R^{13*} are independently selected from a bond, —C(O)—, —C(O)O—, —O—, —S—, —C(O)NR^{b*}—, and —NR^{b*}—; M_{1*}, R^{10*}, R^{11*}, R^{20*}, and R^{b*} are as defined above.

In some embodiments, R^{1*}, R^{2*}, R^{3*}, and R^{5*} are independently selected from

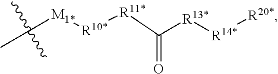

wherein M_{1*}, R^{10*}, R^{11*}, R^{13*}, R^{14*}, and R^{20*} are as defined above.

In some embodiments, R^{1*}, R^{2*}, R^{3*}, and R^{5*} are independently selected from

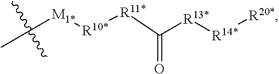

wherein R^{11*} and R^{13*} are independently selected from bond, —C(O)—, —C(O)O—, —O—, —S—, —C(O)NR^{b*}—, and —NR^{b*}—; M_{1*}, R^{10*}, R^{14*}, R^{20*}, and R^{b*} are as defined above.

In some embodiments, M_{1*} is selected from bond, —NH—, —CH₂—, —CH₂CH₂—, —C(O)—, —C(O)O—, —O—, —S—, and —C(O)NH—.

In some embodiments, M_{1*} is selected from —CH₂— and —CH₂CH₂—.

In some embodiments, R^{10*}, R^{11*}, R^{12*}, R^{13*}, and R^{14*} are independently selected from bond, —C(O)—, —C(O)O—, —O—, —S—, —C(O)NR^{b*}—, —NR^{b*}—, 2- to 10-membered heteroalkylene, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R^{a*}.

In some embodiments, R^{20*} is selected from H, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a*}.

In some embodiments, R^{20*} is selected from H, C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, and 5- to 10-membered heteroaryl, wherein the C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, or 5- to 10-membered heteroaryl is optionally substituted with R^{a*}.

In some embodiments, R^{1*} and R^{3*}, together with the atoms linked thereto, form 5-, 6-, 7-, or 8-membered heterocyclic ring, wherein the 5-, 6-, 7-, or 8-membered heterocyclic ring is optionally substituted with R^{5*}.

In some embodiments, R^{1*} and R^{3*}, together with the atoms linked thereto, form 5-, 6-, 7-, or 8-membered heterocyclic ring, wherein the 5-, 6-, 7-, or 8-membered heterocyclic ring contains 1, 2, or 3 heteroatoms or heteroatom groups selected from N, O, and S, and the 5-, 6-, 7-, or 8-membered heterocyclic ring is optionally substituted with R^{5*}.

In some embodiments, R^{4*} is independently selected from halogen, CN, OH, NH₂, 2- to 10-membered heteroalkyl, and C₁-C₁₀ alkyl, wherein the OH, NH₂, 2- to 10-membered heteroalkyl, or C₁-C₁₀ alkyl is optionally substituted with R^{a*}.

In some embodiments, each R^{a*} is independently selected from halogen, CN, OH, NH₂, C₁-C₁₀ alkyl, C₃-C₁₀ cycloalkyl, and 4- to 8-membered heterocyclyl, wherein the OH, NH₂, C₁-C₁₀ alkyl, C₃-C₁₀ cycloalkyl, or 4- to 8-membered heterocyclyl is optionally substituted with R^{c*}.

In some embodiments, each R^{a*} is independently selected from halogen, CN, OH, NH₂, and C₁-C₁₀ alkyl, wherein the OH, NH₂, or C₁-C₁₀ alkyl is optionally substituted with R^{c*}.

In some embodiments, each R^{a*} is independently selected from halogen and C₁-C₆ alkyl, wherein the C₁-C₆ alkyl is optionally substituted with R^{c*}.

In some embodiments, each R^{a*} is independently selected from F, Cl, CH₃, and CF₃.

In some embodiments, each R^{b*} is independently selected from H, C₁-C₆ alkyl, C₃-C₁₀ cycloalkyl, 4- to 8-membered heterocyclyl, C₆-C₁₀ aryl, and 5- to 10-membered heteroaryl, wherein the C₁-C₆ alkyl, C₃-C₁₀ cycloalkyl, 4- to 8-membered heterocyclyl, C₆-C₁₀ aryl, or 5- to 10-membered heteroaryl is optionally substituted with R^{c*}.

In some embodiments, each R^{b*} is independently selected from H and C₁-C₆ alkyl, wherein the C₁-C₆ alkyl is optionally substituted with R^{c*}.

In some embodiments, each R^{c*} is independently selected from halogen, CN, OH, NH₂, and C₁-C₆ alkyl.

In some embodiments, m* and p* are independently selected from 0, 1, 2, 3, and 4.

In some embodiments, m* and p* are independently selected from 1, 2, 3, and 4.

In some embodiments, m* and p* are independently selected from 1 and 2.

In some embodiments, n* is selected from 0 and 1.

In some embodiments, n* is selected from 0.

In some embodiments, the compound represented by formula (V) or the pharmaceutically acceptable salt thereof of the present disclosure is selected from a compound represented by formula (VI) or a pharmaceutically acceptable salt thereof,

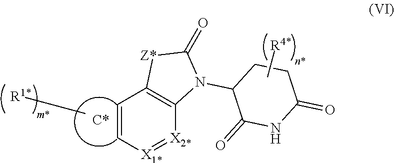

wherein X_{1*} and X_{2*} are independently selected from N and CH, wherein the CH is optionally substituted with R^{2*}; ring C, Z*, R^{1*}, R^{2*}, R^{4*}, m*, and n* are as defined above.

In some embodiments, the compound represented by formula (VI) or the pharmaceutically acceptable salt thereof of the present disclosure is selected from a compound represented by formula (VI-1a) or formula (VI-1b) or a pharmaceutically acceptable salt thereof,

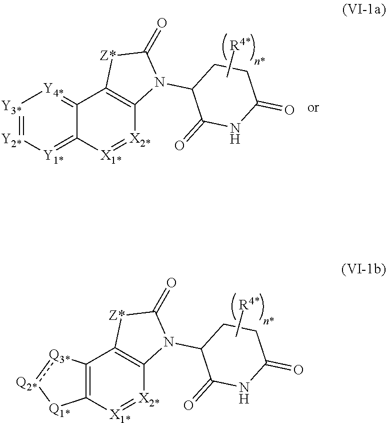

wherein X_{1*} and X_{2*} are independently selected from N and CH, wherein the CH is optionally substituted with R^{2*}; Y_{1*}, Y_{2*}, Y_{3*}, and Y_{4*} are independently selected from N and CH, wherein the CH is optionally substituted with R^{1*}; Q_{1*}, Q_{2*}, and Q_{3*} are independently selected from O, S, NH, CH₂, N, and CH, wherein the NH, CH₂, or CH is optionally substituted with R¹*; Z*, R^{1*}, R^{2*}, R^{4*}, and n* are as defined above.

Without conflict, it should be understood that the embodiments described above may be combined arbitrarily, forming technical solutions that include the features of the combined embodiments. Such combined technical solutions are within the scope of the present disclosure.

In some embodiments, the compound represented by formula (III) or formula (IV) or formula (V) or the pharmaceutically acceptable salt thereof is selected from the following compounds or pharmaceutically acceptable salts thereof,

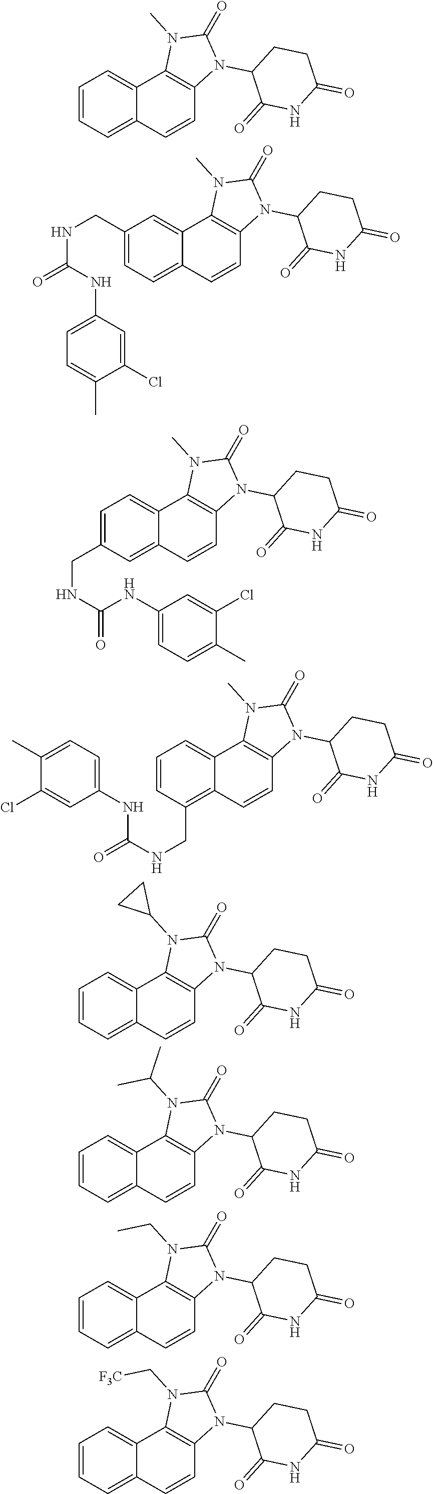

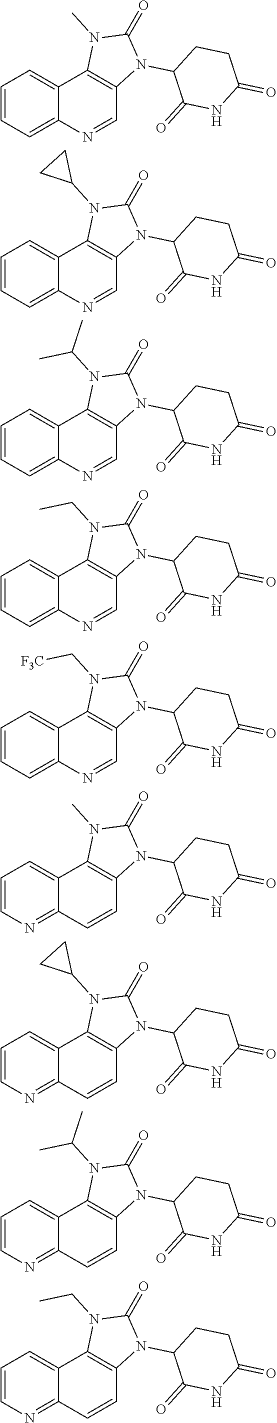

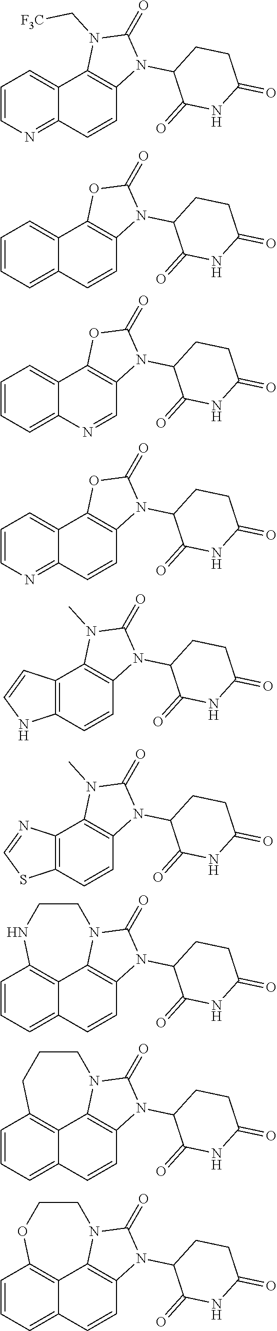

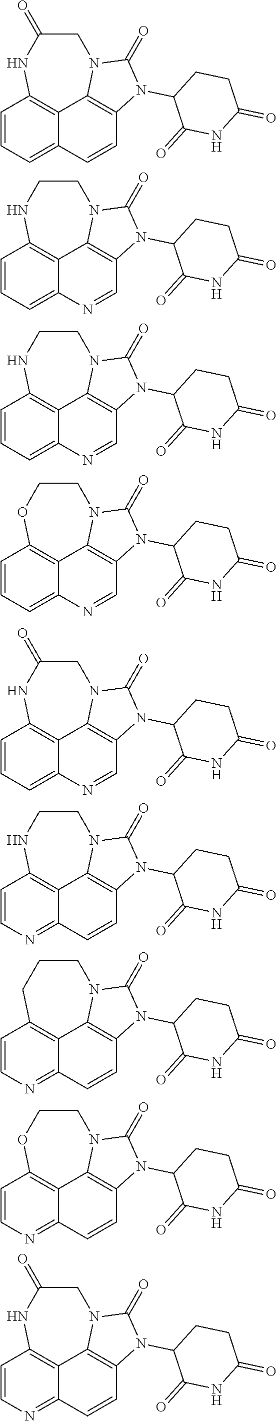

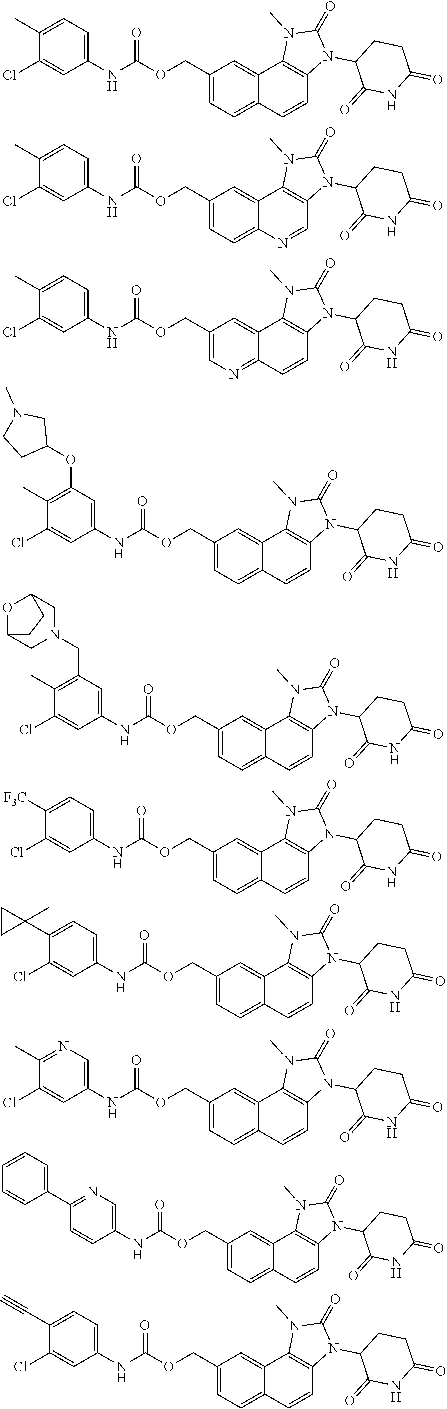

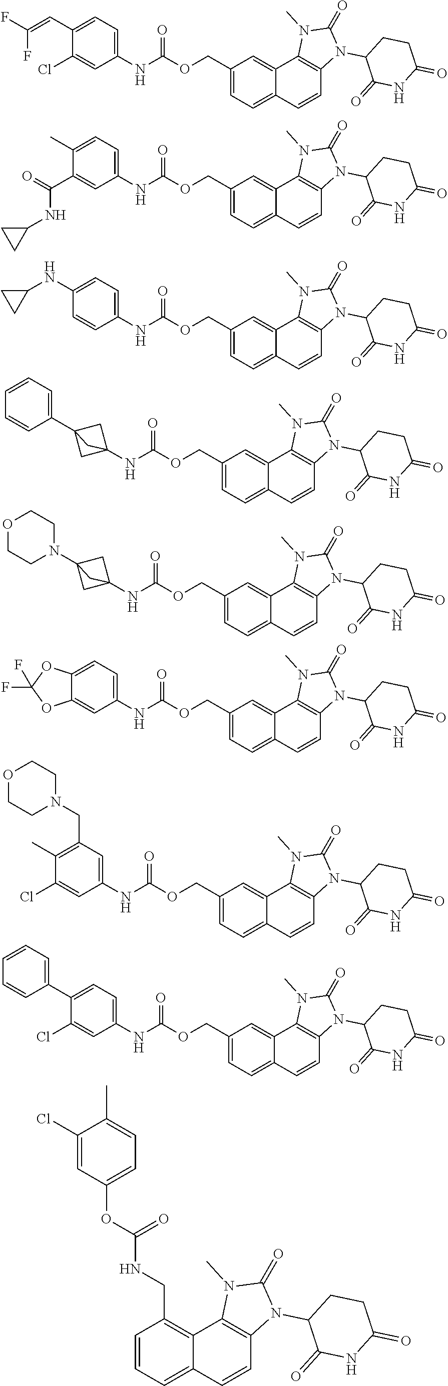

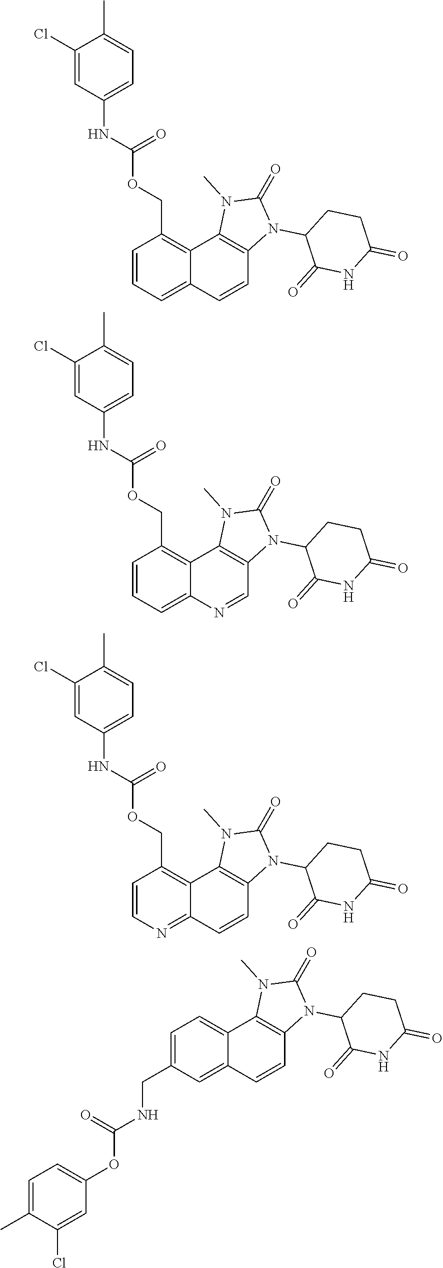

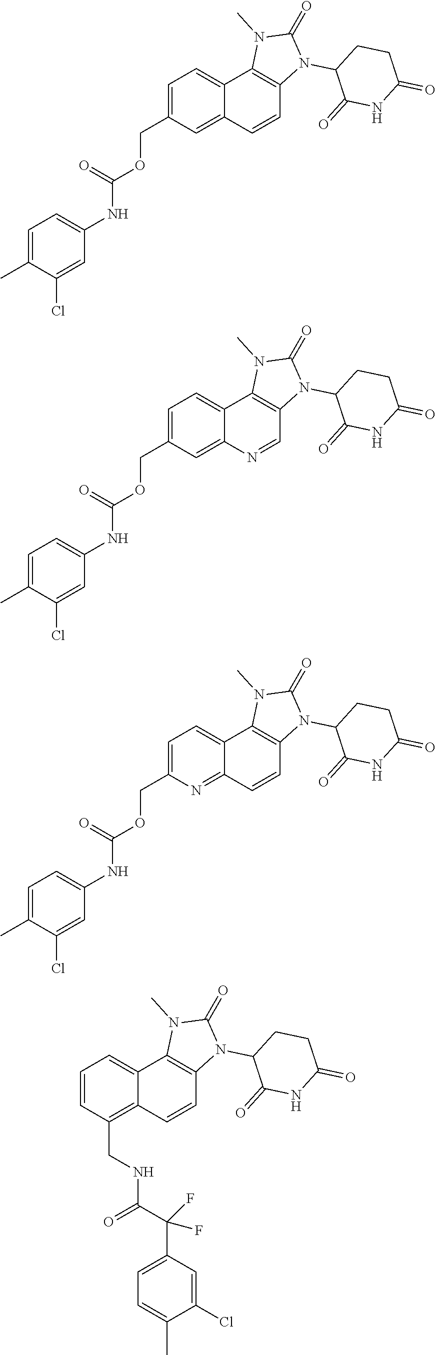 [SMILES: Cc1ccc(NC(=O)OCc2ccc3c(ccc4c3n(C)c(=O)n4C3CCC(=O)NC3=O)c2)cc1Cl ; Cc1ccc(NC(=O)OCc2ccc3c(c2)ncc2c3n(C)c(=O)n2C2CCC(=O)NC2=O)cc1Cl ; Cc1ccc(NC(=O)OCc2ccc3c(ccc4c3n(C)c(=O)n4C3CCC(=O)NC3=O)n2)cc1Cl ; Cc1ccc(C(F)(F)C(=O)NCc2cccc3c2ccc2c3n(C)c(=O)n2C2CCC(=O)NC2=O)cc1Cl]

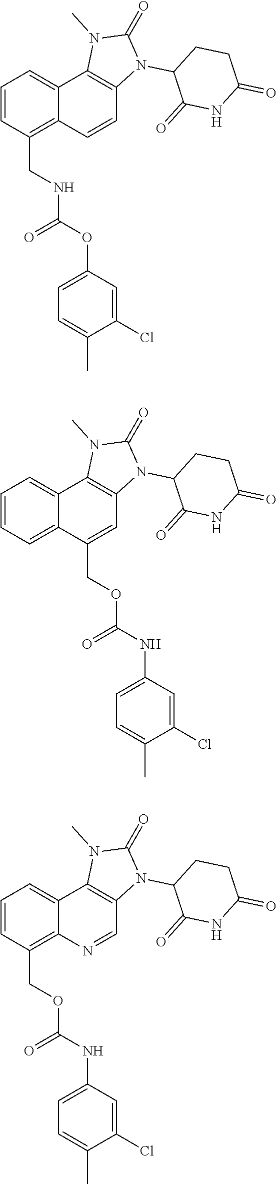

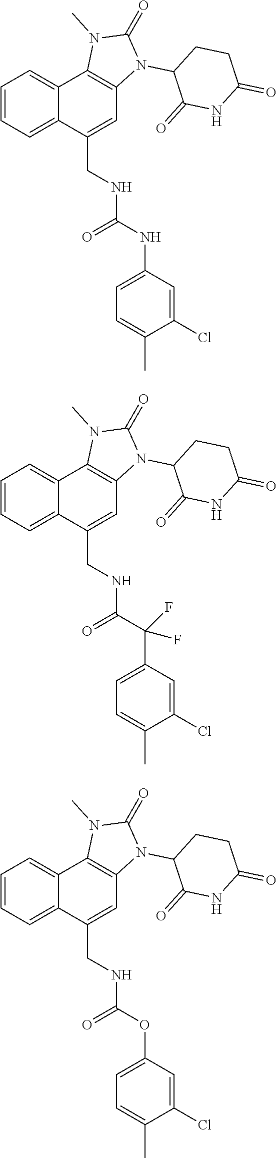

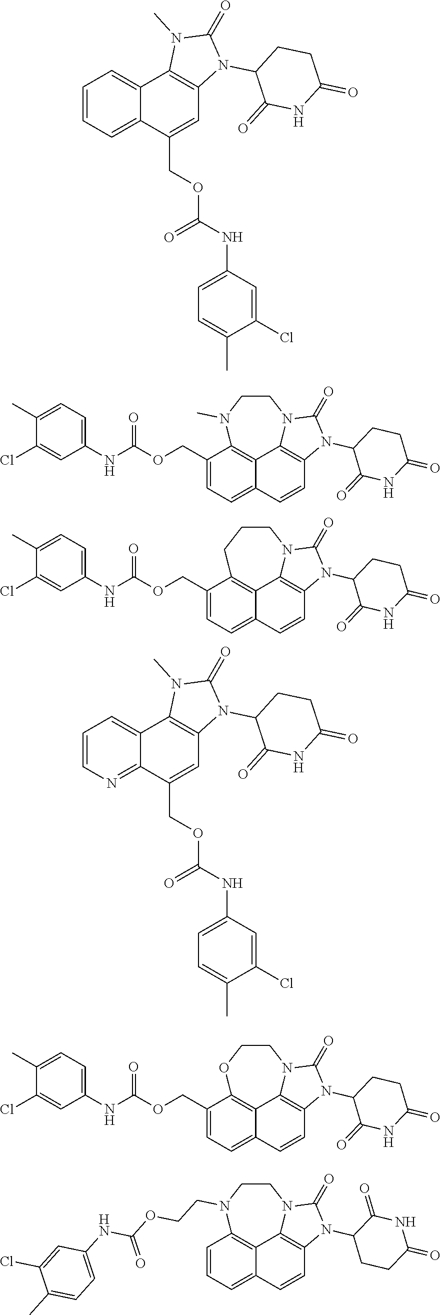

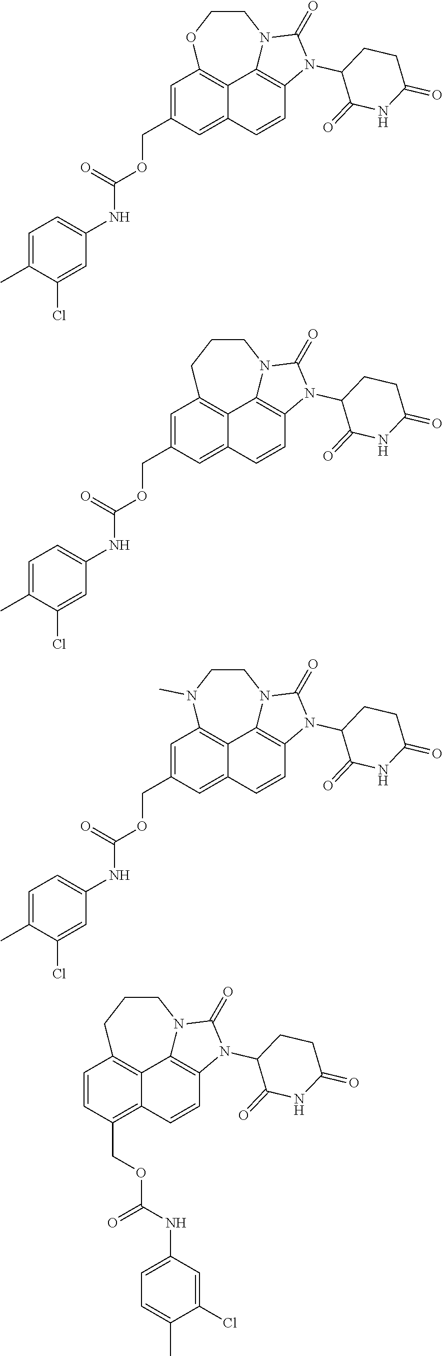 [SMILES: Cc1ccc(NC(=O)OCc2cc3c4c(ccc5c4n(c(=O)n5C4CCC(=O)NC4=O)CCO3)c2)cc1Cl ; Cc1ccc(NC(=O)OCc2cc3c4c(ccc5c4n(c(=O)n5C4CCC(=O)NC4=O)CCC3)c2)cc1Cl ; Cc1ccc(NC(=O)OCc2cc3c4c(ccc5c4n(c(=O)n5C4CCC(=O)NC4=O)CCN3C)c2)cc1Cl ; Cc1ccc(NC(=O)OCc2ccc3c4c2ccc2c4n(c(=O)n2C2CCC(=O)NC2=O)CCC3)cc1Cl]

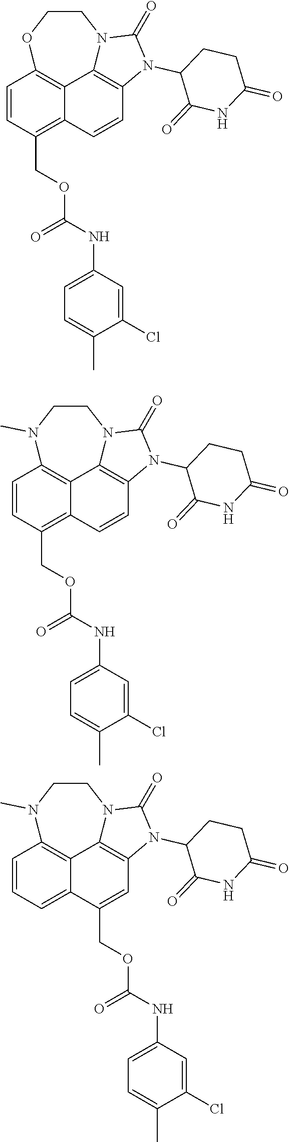

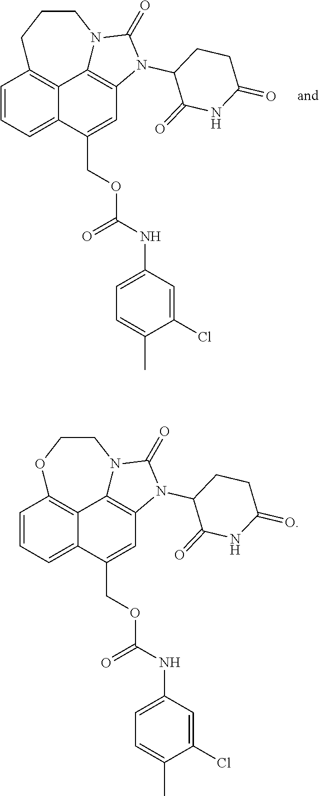 [SMILES: Cc1ccc(NC(=O)OCc2cc3c4c5c(cccc25)CCCn4c(=O)n3C2CCC(=O)NC2=O)cc1Cl ; Cc1ccc(NC(=O)OCc2cc3c4c5c(cccc25)OCCn4c(=O)n3C2CCC(=O)NC2=O)cc1Cl]

In another aspect, the present disclosure provides a pharmaceutical composition comprising the compound represented by formula (V) or the pharmaceutically acceptable salt thereof of the present disclosure and a pharmaceutically acceptable excipient.

In another aspect, the present disclosure provides a method for treating an abnormal cell proliferation disease in a mammal, which comprises administering to a mammal, preferably a human, in need of the treatment a therapeutically effective amount of the compound represented by formula (V) or the pharmaceutically acceptable salt thereof, or the pharmaceutical composition thereof.

In another aspect, the present disclosure provides use of the compound represented by formula (V) or the pharmaceutically acceptable salt thereof, or the pharmaceutical composition thereof in the preparation of a medicament for preventing or treating an abnormal cell proliferation disease.

In another aspect, the present disclosure provides use of the compound represented by formula (V) or the pharmaceutically acceptable salt thereof, or the pharmaceutical composition thereof in the prevention or treatment of an abnormal cell proliferation disease.

In another aspect, the present disclosure provides the compound represented by formula (V) or the pharmaceutically acceptable salt thereof, or the pharmaceutical composition thereof for use in preventing or treating an abnormal cell proliferation disease.

In some embodiments, the abnormal cell proliferation disease is selected from cancers.

In some embodiments, the cancer is selected from a solid tumor, an adenocarcinoma, and a hematologic tumor.

In another aspect, the present disclosure also provides use of the compound represented by formula (III), formula (IV), or formula (V) or the pharmaceutically acceptable salt thereof in the preparation of a medicament for targeted protein degradation.

In another aspect, the present disclosure also provides use of the compound represented by formula (III), formula (IV), or formula (V) or the pharmaceutically acceptable salt thereof as an intermediate in the preparation of a medicament for targeted protein degradation.

Without conflict, it should be understood that the embodiments described above may be combined arbitrarily, forming technical solutions that include the features of the combined embodiments. Such combined technical solutions are within the scope of the present disclosure.

### TERMINOLOGY AND DEFINITIONS

Unless otherwise stated, the terms used in the present disclosure have the following meanings, and the definitions of groups and terms described in the present disclosure, including definitions thereof as examples, exemplary definitions, preferred definitions, definitions documented in tables, definitions of specific compounds in the examples, and the like, may be arbitrarily combined and incorporated with each other. A certain term, unless otherwise specifically defined, should not be considered uncertain or unclear, but should be understood according to its common meaning in the field. When referring to a trade name, it is intended to refer to its corresponding commercial product or its active ingredient.

Herein,

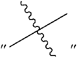

represents a connection site. For

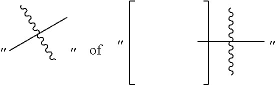

used herein, when

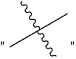

is not connected to a fixed ring or atom, it indicates that

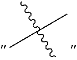

can connect to a group resulting from the loss of a hydrogen atom at any site within the structure denoted by “[ ]” that contains the substitutable hydrogen atom (including hydrogen atoms directly connected to ring atoms, hydrogen atoms on non-hydrogen substituents of ring atoms, and hydrogen atoms on further substituents of those substituents). For example, the connection sites for

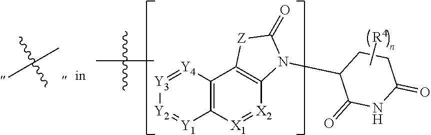

include, but are not limited to, Y¹, Y², Y³, Y⁴, X¹, X², Z, substituents thereof, etc.

The illustration method for the racemic or enantiomerically pure compounds herein is from Maehr, J. Chem. Ed. 1985, 62:114-120. Unless otherwise stated, the absolute configuration of a stereogenic center is represented by a wedged bond and a wedged dashed bond ( [symbol] and [symbol] ), and the relative configuration of a stereogenic center (e.g., cis- or trans-configuration of alicyclic compounds) is represented by a black solid bond and a dashed bond ( [symbol] and [symbol] ).

The term “tautomer” refers to functional isomers resulting from the rapid movement of an atom in a molecule between two positions. The compounds of the present disclosure may exhibit the tautomerism. Tautomeric compounds may exist in two or more interconvertible forms. Tautomers generally exist in an equilibrium form. Trying to separate a single tautomer usually leads to a mixture, the physicochemical properties of which are consistent with the mixture of the compound. The position of the equilibrium depends on the chemical properties of the molecule. For example, in many aliphatic aldehydes and ketones such as acetaldehyde, the keto form predominates; whereas in phenol, the enol form predominates. In the present disclosure, all tautomeric forms of the compounds are included.

The term “stereoisomer” refers to isomers resulting from different spatial arrangements of atoms in a molecule, including cis-trans isomers, enantiomers, and diastereoisomers.

The compounds of the present disclosure may have an asymmetric atom such as a carbon atom, a sulfur atom, a nitrogen atom, and a phosphorus atom, or an asymmetric double bond, and thus the compounds of the present disclosure may exist in the form of a particular geometric isomer or stereoisomer. The form of a particular geometric isomer or stereoisomer may be cis and trans isomers, E and Z geometric isomers, (−)- and (+)-enantiomers, (R)- and (S)-enantiomers, diastereoisomers, (D)-isomers, (L)-isomers, and racemic mixtures or other mixtures thereof, such as an enantiomer or diastereoisomer enriched mixture, and all of the above isomers, as well as mixtures thereof, are encompassed within the definition scope of the compounds of the present disclosure. An additional asymmetric carbon atom, asymmetric sulfur atom, asymmetric nitrogen atom, or asymmetric phosphorus atom may be present in substituents such as alkyl. All of these isomers and mixtures thereof referred to in the substituents are also encompassed within the definition scope of the compounds of the present disclosure. The compounds containing asymmetric atoms of the present disclosure can be separated in an optically active pure form or in a racemic form. The optically active pure form can be obtained by resolving a racemic mixture or by synthesis using chiral starting materials or chiral reagents.

The term “substituted” means that any one or more hydrogen atoms on a specific atom are substituted with substituents, as long as the valence of the specific atom is normal and the compound resulting from the substitution is stable. When the substituent is oxo (namely ═O), it means that two hydrogen atoms are substituted, and oxo is not available in an aromatic group.

The term “optional” or “optionally” means that the subsequently described event or circumstance may, but not necessarily, occur. The description includes instances where the event or circumstance occurs and instances where the event or circumstance does not. For example, ethyl being “optionally” substituted with halogen means that the ethyl may be unsubstituted (CH₂CH₃), monosubstituted (CH₂CH₂F, CH₂CH₂Cl, or the like), polysubstituted (CHFCH₂F, CH₂CHF₂, CHFCH₂Cl, CH₂CHCl₂, or the like), or fully substituted (CF₂CF₃, CF₂CCl₃, CCl₂CCl₃, or the like). It will be understood by those skilled in the art that for any group comprising one or more substituents, no substitution or substituting pattern that is spatially impossible and/or cannot be synthesized will be introduced.

When any variable (e.g., R^{a} or R^{b}) occurs more than once in the constitution or structure of a compound, the variable is independently defined in each case. For example, if a group is substituted with two R^{b}, the definition of each R^{b} is independent.

When one of variables is selected from a chemical bond or is absent, it means that the two groups which it links are linked directly. For example, when L in A-L-Z represents a bond, it means that the structure is actually A-Z.

When the linking direction of the linking group referred to herein is not specified, the linking direction is arbitrary. For example, when L¹ in the structural unit

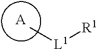

is selected from “C₁-C₃ alkylene-O”, then L¹ may link ring Q and R¹ in a direction from left to right to constitute “ring Q-C₁-C₃ alkylene-O—R¹”, or link ring Q and R¹ in a direction from right to left to constitute “ring Q-O—C₁-C₃ alkylene-R¹”.

When a bond of a substituent is cross-linked to two atoms on a ring, the substituent can be bonded to any atom on the ring. For example, the structural unit

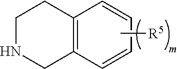

represents that R⁵ may be substituted at any position on the benzene ring.

Cₘ-Cₙ used herein means that the portion has an integer number of carbon atoms in the range of m-n. For example, “C₁-C₁₀” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

Herein, the bond “ [symbol] ” depicted by a solid line and a dotted line represents a single bond or a double bond. For example, the structural unit

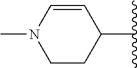

comprises

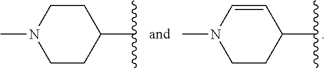

The term “alkyl” refers to a hydrocarbon group with a general formula of CₙH₂ₙ₊₁. The alkyl may be linear or branched. The term “C₁₋₁₀ alkyl” may be understood to represent a linear or branched saturated hydrocarbon group having 1, 2, 3, 4, 5, 6, 7, 8, 9, or 10 carbon atoms. Specific examples of the alkyl include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, and the like. The term “C₁₋₆ alkyl” may be understood to represent an alkyl group having 1, 2, 3, 4, 5, or 6 carbon atoms. Specific examples include, but are not limited to, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like. The term “C₁-C₃ alkyl” may be understood to represent a linear or branched saturated alkyl group having 1, 2, or 3 carbon atoms. The “C₁-C₁₀ alkyl” may include the ranges of “C₁-C₆ alkyl”, “C₁-C₃ alkyl”, and the like, and the “C₁-C₆ alkyl” may further include “C₁-C₃ alkyl”.

The term “alkylene” refers to a divalent group derived from an “alkyl” group as defined herein.

The term “heteroalkyl” refers to an alkyl group containing 1, 2, 3, 4, or 5 heteroatoms or heteroatom groups, including but not limited to, N, O, S, B, P, —S(═O)₂—, —S(═O)—, —NH—, and the like. The term “2- to 10-membered heteroalkyl” may be understood to represent a heteroalkyl group having 2, 3, 4, 5, 6, 7, 8, 9, or 10 atoms (carbon and heteroatoms other than hydrogen). The term “2- to 6-membered heteroalkyl” may be understood to represent a heteroalkyl group having 2, 3, 4, 5, or 6 atoms (carbon and heteroatoms other than hydrogen). The heteroalkyl may be linked to other groups through heteroatoms or carbon atoms therein. The heteroatom may be located at any internal position of the heteroalkyl (including the position at which the heteroalkyl is linked to other groups), i.e., the heteroalkyl does not include hydroxyalkyl (e.g., —CH₂OH or —CH(CH₃)OH), aminoalkyl (e.g., —CH₂NH₂ or —CH(CH₃)NH₂), and the like. Examples of the heteroalkyl include, but are not limited to, —OCH₃, —OCH₂CH₃, —OCH₂(CH₃)₂, —CH₂—CH₂—O—CH₃, —NHCH₃, —N(CH₃)₂, —NHCH₂CH₃, —CH₂—CH₂—NH—CH₃, —OCH₂—CH₂—NH—CH₃, —OCH₂—CH₂—NH—CH(CH₃)₂, —SCH₃, —SCH₂CH₃, —S(═O) —CH₃, —CH₂—S(═O)₂—CH₃, —CH₂—C(═O)NH—CH₂—O—CH₃.

The term “heteroalkylene” refers to a divalent group derived from a “heteroalkyl” group as defined herein.

The term “alkenyl” refers to a linear or branched unsaturated aliphatic hydrocarbon group consisting of carbon atoms and hydrogen atoms and having at least one double bond. The term “C₂-C₁₀ alkenyl” may be understood to represent a linear or branched unsaturated hydrocarbyl group comprising one or more double bonds and having 2, 3, 4, 5, 6, 7, 8, 9, or 10 carbon atoms, and the “C₂-C₁₀ alkenyl” is preferably “C₂-C₆ alkenyl”, further preferably “C₂-C₄ alkenyl”, and still further preferably “C₂ or C₃ alkenyl”. It can be understood that in the case that the alkenyl comprises more than one double bond, the double bonds can be separated from one another or conjugated. Specific examples of the alkenyl include, but are not limited to, vinyl, allyl, (E)-2-methylvinyl, (Z)-2-methylvinyl, (E)-but-2-enyl, (Z)-but-2-enyl, (E)-but-1-enyl, (Z)-but-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, and the like.

The term “alkenylene” refers to a divalent group derived from an “alkenyl” group as defined herein.

The term “alkynyl” refers to a linear or branched unsaturated aliphatic hydrocarbon group consisting of carbon atoms and hydrogen atoms and having at least one triple bond. The term “C₂-C₁₀ alkynyl” may be understood to represent a linear or branched unsaturated hydrocarbyl group comprising one or more triple bonds and having 2, 3, 4, 5, 6, 7, 8, 9, or 10 carbon atoms. Examples of the “C₂-C₁₀ alkynyl” include, but are not limited to, ethynyl (—C≡CH), propynyl (—C≡CCH₃ or —CH₂C≡CH), but-1-ynyl, but-2-ynyl, and but-3-ynyl. The “C₂-C₁₀ alkynyl” may include “C₂-C₃ alkynyl”, and examples of the “C₂-C₃ alkynyl” include ethynyl (—C≡CH), prop-1-ynyl (—C≡CCH₃), and prop-2-ynyl (—CH₂C≡CH).

The term “alkynylene” refers to a divalent group derived from an “alkynyl” group as defined herein.

The term “cycloalkyl” refers to a fully saturated carbon ring that exists in the form of a monocycle ring, fused ring, bridged ring, spiro ring, and the like. Unless otherwise specified, the carbon ring is generally a 3- to 10-membered ring. The term “C₃-C₁₀ cycloalkyl” may be understood to represent a saturated monocyclic ring, fused ring, spiro ring, or bridged ring having 3, 4, 5, 6, 7, 8, 9, or 10 carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo [2.2.1]heptyl), bicyclo[2.2.2]octyl, adamantyl, spiro[4.5]decyl, and the like. The term “C₃-C₁₀ cycloalkyl” may include “C₃-C₆ cycloalkyl”, and the term “C₃-C₆ cycloalkyl” may be understood to represent a saturated monocyclic or bicyclic hydrocarbon ring having 3, 4, 5, or 6 carbon atoms.

Specific examples include, but are not limited to, cyclopropyl, cyclobutyl, cyclopentyl, cyclohexyl, and the like. The term “C₅-C₈ cycloalkyl” may be understood to represent a saturated monocyclic or bicyclic hydrocarbon ring having 5, 6, 7, or 8 carbon atoms, and may also represent a C₅-C₈ saturated carbon ring.

The term “cycloalkylene” refers to a divalent group derived from a “cycloalkyl” group as defined herein.

The term “heterocyclyl” refers to a fully saturated or partially saturated (non-aromatic heteroaromatic group on the whole) monocyclic ring, fused ring, spiro ring, or bridged ring group, and ring atoms of the group include 1, 2, 3, 4, or 5 heteroatoms or heteroatom groups (i.e., heteroatom-containing atom groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —P(═O)₂—, —P(═O)—, —NH—, —S(═O)(═NH)—, —C(═O)NH—, —NHC(═O)NH—, and the like. The term “3- to 10-membered heterocyclyl” refers to a heterocyclyl group with 3, 4, 5, 6, 7, 8, 9, or 10 ring atoms, and ring atoms of the group include 1, 2, 3, 4, or 5 heteroatoms or heteroatom groups independently selected from those described above. “3- to 10-membered heterocyclyl” includes “4- to 7-membered heterocyclyl”, wherein specific examples of 4-membered heterocyclyl include, but are not limited to, azetidinyl, thietanyl, and oxetanyl; specific examples of 5-membered heterocyclyl include, but are not limited to, tetrahydrofuranyl, dioxolyl, pyrrolidinyl, imidazolidinyl, pyrazolidinyl, pyrrolinyl, 4,5-dihydrooxazolyl, and 2,5-dihydro-TH-pyrrolyl; specific examples of 6-membered heterocyclyl include, but are not limited to, tetrahydropyranyl, piperidyl, morpholinyl, dithianyl, thiomorpholinyl, piperazinyl, trithianyl, tetrahydropyridinyl, and 4H-[1,3,4]thiadiazinyl; specific examples of 7-membered heterocyclyl include, but are not limited to, diazepanyl. The heterocyclyl may also be a bicyclic group, wherein specific examples of 5,5-membered bicyclic groups include, but are not limited to, hexahydrocyclopenta[c]pyrrol-2(1H)-yl; specific examples of 5,6-membered bicyclic groups include, but are not limited to, hexahydropyrrolo[1,2-a]pyrazin-2(1H)-yl, 5,6,7,8-tetrahydro-[1,2,4]triazolo[4,3-a]pyrazinyl, and 5,6,7,8-tetrahydroimidazo[1,5-a]pyrazinyl. Optionally, the heterocyclyl may be a benzo-fused ring group of the 4- to 7-membered heterocyclyl described above. Specific examples include, but are not limited to, dihydroisoquinolyl and the like. “4- to 10-membered heterocyclyl” may include the ranges of “5- to 10-membered heterocyclyl”, “4- to 7-membered heterocyclyl”, “5- to 6-membered heterocyclyl”, “6- to 8-membered heterocyclyl”, “4- to 10-membered heterocycloalkyl”, “5- to 10-membered heterocycloalkyl”, “4- to 7-membered heterocycloalkyl”, “5- to 6-membered heterocycloalkyl”, “6- to 8-membered heterocycloalkyl”, and the like, and the “4- to 7-membered heterocyclyl” may further include the ranges of “4- to 6-membered heterocyclyl”, “5- to 6-membered heterocyclyl”, “4- to 7-membered heterocycloalkyl”, “4- to 6-membered heterocycloalkyl”, “5- to 6-membered heterocycloalkyl”, and the like. Although some bicyclic heterocyclyl groups herein comprise, in part, one benzene ring or one heteroaromatic ring, the heterocyclyl is still non-aromatic on the whole.

The term “heterocyclylene” refers to a divalent group derived from a “heterocyclyl” group as defined herein.

The term “aryl” refers to an aromatic all-carbon monocyclic or fused polycyclic group with a conjugated π-electron system. Aryl may have 6-20 carbon atoms, 6 to 14 carbon atoms, or 6 to 12 carbon atoms. The term “C₆-C₂₀ aryl” may be understood as an aryl group having 6 to 20 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl, or a ring having 13 carbon atoms (“C₁₃ aryl”), such as fluorenyl, or a ring having 14 carbon atoms (“C₁₄ aryl”), such as anthracenyl. The term “C₆-C₁₀ aryl” may be understood as an aryl group having 6 to 10 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl. The term “C₆-C₂₀ aryl” may include “C₆-C₁₀ aryl”.

The term “arylene” refers to a divalent group derived from an “aryl” group as defined herein.

The term “heteroaryl” refers to an aromatic cyclic group having an aromatic monocyclic or fused polycyclic system, which contains at least one ring atom selected from N, O, and S, with the remaining ring atoms being C. The term “5- to 10-membered heteroaryl” may be understood to include an aromatic monocyclic or bicyclic ring system, which has 5, 6, 7, 8, 9, or 10 ring atoms, particularly 5, 6, 9, or 10 ring atoms, and comprises 1, 2, 3, 4, or 5, preferably 1, 2, or 3, heteroatoms independently selected from N, O, and S. In particular, the heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, and the like, and benzo derivatives thereof, such as benzofuranyl, benzothienyl, benzothiazolyl, benzoxazolyl, benzoisoxazolyl, benzimidazolyl, benzotriazolyl, indazolyl, indolyl, isoindolyl, and the like; and pyridyl, pyridazinyl, pyrimidinyl, pyrazinyl, triazinyl, and the like, and benzo derivatives thereof, such as quinolyl, quinazolinyl, isoquinolyl, and the like; and azocinyl, indolizinyl, purinyl, and the like, and benzo derivatives thereof; and cinnolinyl, phthalazinyl, quinazolinyl, quinoxalinyl, naphthyridinyl, pteridinyl, carbazolyl, acridinyl, phenazinyl, phenothiazinyl, phenoxazinyl, and the like. The term “5- to 6-membered heteroaryl” refers to an aromatic ring system, which has 5 or 6 ring atoms and comprises 1, 2, or 3, preferably 1-2, heteroatoms independently selected from N, O, and S.

The term “heteroarylene” refers to a divalent group derived from a “heteroaryl” group as defined herein.

The term “halo” or “halogen” refers to fluorine, chlorine, bromine, or iodine.

The term “therapeutically effective amount” means:
an amount of the compound of the present disclosure for (i) treating a specific disease, condition, or disorder; (ii) alleviating, ameliorating, or eliminating one or more symptoms of a specific disease, condition, or disorder; or (iii) delaying the onset of the one or more symptoms of the specific disease, condition, or disorder described herein.

The amount of the compound of the present disclosure constituting the “therapeutically effective amount” varies depending on the compound, the disease state and its severity, the administration regimen, and the age of the mammal to be treated, but can be determined routinely by those skilled in the art in accordance with their knowledge and the present disclosure.

The term “pharmaceutically acceptable” is used herein for those compounds, materials, compositions, and/or dosage forms that are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to salts of pharmaceutically acceptable acid addition or base addition salts, including salts formed from the compound and an inorganic or organic acid, and salts formed from the compound and an inorganic or organic base.

The term “pharmaceutical composition” refers to a mixture consisting of one or more of the compounds or the salts thereof of the present disclosure and a pharmaceutically acceptable excipient.

The pharmaceutical composition is intended to facilitate the administration of the compound of the present disclosure to an organism.

The term “pharmaceutically acceptable excipient” refers to those that do not have a significant irritating effect on an organism and do not impair the biological activity and properties of the active compound. Suitable excipients are well known to those skilled in the art, such as carbohydrate, wax, water-soluble and/or water-swellable polymers, hydrophilic or hydrophobic materials, gelatin, oil, solvent, water, and the like.

The word “comprise” and variations thereof such as “comprises” or “comprising” may be understood in an open and non-exclusive sense, i.e., “including but not limited to”.

The present disclosure also includes isotopically labeled compounds of the present disclosure which are identical to those documented herein but have one or more atoms replaced by an atom having an atomic mass or mass number different from the atomic mass or mass number usually found in nature.

Examples of isotopes that can be incorporated into the compounds of the present disclosure include isotopes of hydrogen, carbon, nitrogen, oxygen, phosphorus, sulfur, fluorine, iodine, and chlorine, such as ²H, ³H, ¹¹C, ¹³C, ⁴C, ¹³N, ¹⁵N, ¹⁵O, ¹⁷O, ¹⁸O, ³¹P, ³²P. ³⁵S, ¹⁸F, ¹²³I, ¹²⁵I, and ³⁶Cl.

Certain isotopically labeled compounds of the present disclosure (e.g., those labeled with ³H and ¹⁴C) can be used to analyze compounds and/or substrate tissue distribution. Tritiated (i.e., ³H) and carbon-14 (i.e., ¹⁴C) isotopes are particularly preferred for their ease of preparation and detectability. Positron emitting isotopes, such as ¹⁵O, ¹³N, ¹¹C, and ¹⁸F, can be used in positron emission tomography (PET) studies to determine substrate occupancy. Isotopically labeled compounds of the present disclosure can generally be prepared by following procedures analogous to those disclosed in the schemes and/or examples below while substituting a non-isotopically labeled reagent with an isotopically labeled reagent.

The pharmaceutical composition of the present disclosure can be prepared by combining the compound of the present disclosure with a suitable pharmaceutically acceptable excipient, and can be formulated, for example, into a solid, semisolid, liquid, or gaseous formulation such as tablet, pill, capsule, powder, granule, ointment, emulsion, suspension, suppository, injection, inhalant, gel, microsphere, aerosol, and the like.

Typical routes of administration of the compound or the pharmaceutically acceptable salt thereof or the pharmaceutical composition thereof of the present disclosure include, but are not limited to, oral, rectal, local, inhalation, parenteral, sublingual, intravaginal, intranasal, intraocular, intraperitoneal, intramuscular, subcutaneous, and intravenous administration.

The pharmaceutical composition of the present disclosure can be manufactured by methods well known in the art, such as conventional methods of mixing, dissolving, granulating, emulsifying, lyophilizing, and the like.

In some embodiments, the pharmaceutical composition is in an oral form. For oral administration, the pharmaceutical composition can be formulated by mixing the active compounds with pharmaceutically acceptable excipients well known in the art. These excipients enable the compounds of the present disclosure to be formulated into tablets, pills, lozenges, dragees, capsules, liquids, gels, slurries, suspensions, and the like, for oral administration to patients.

A solid oral composition can be prepared by conventional mixing, filling, or tableting. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to: binders, diluents, disintegrants, lubricants, glidants, flavoring agents, and the like.

The pharmaceutical compositions may also be suitable for parenteral administration, such as sterile solutions, suspensions, or lyophilized products in suitable unit dosage forms.

In all of the administration methods of the compound represented by general formula (I) described herein, the daily administration dose is from 0.01 mg/kg to 200 mg/kg of body weight, given in individual or separated doses.

### DETAILED DESCRIPTION

The present disclosure will be described in detail with reference to examples, but the following examples should not be construed as limiting the scope of the present disclosure.

The structures of the compounds are determined by nuclear magnetic resonance (NMR) and/or mass spectrometry (MS). NMR shifts are given in 106 (ppm). The solvents for NMR determination are deuterated dimethyl sulfoxide, deuterated chloroform, deuterated methanol, and the like, and the internal standard is tetramethylsilane (TMS); “IC₅₀” refers to the half maximal inhibitory concentration, which is the concentration at which half of the maximal inhibitory effect is achieved.

Unless otherwise stated, the ratios expressed for mixed solvents are volume mixing ratios.

Unless otherwise stated, % refers to wt %.

The following eluents may be mixed eluents formed by two or more solvents in a ratio of volume ratios of the respective solvents, such as “water:acetonitrile=1:1” represents that the volume ratio of water to acetonitrile in the mixed eluent is 1:1 during elution.

### Abbreviation

ACN: acetonitrile; TEA: triethylamine; DPPA: diphenylphosphoryl azide; DMF: N,N-dimethylformamide; THF: tetrahydrofuran; HOBT: 1-hydroxybenzotriazole; DCM: dichloromethane; Trifluoroacetamide: trifluoroacetamide; EDCI: 1-(3-dimethylaminopropyl)-3-ethylcarbodiimide hydrochloride; 2-MeTHF: 2-methyltetrahydrofuran; Glyoxal: glyoxal; t-BuOK or KOtBu: potassium tert-butoxide; toluene: toluene; MsOH: methanesulfonic acid; Pd(OAc)₂: palladium(II) acetate; Catacxium: di(1-adamantyl)-n-butylphosphine; Dioxane: 1,4-dioxane; DIEA: N,N-diisopropylethylamine; HATU: O-(7-azabenzotriazol-1-yl)-N,N,N′,N′-tetramethyluronium hexafluorophosphate; NBS: N-bromosuccinimide; Ephos Pd G4: methanesulfonato{dicyclohexyl[3-(1-methylethoxy)-2′,4′,6′-tris(1-methylethyl)-1,1′-biphenyl-2-yl]phosphine}(2′-methylamino-1,1′-biphenyl-2-yl)palladium(II); Ephos: dicyclohexyl(3-isopropoxy-2′,4′,6′-triisopropyl-[1,1′-biphenyl]-2-yl)phosphane; triphosgene: triphosgene; TFA: trifluoroacetic acid; TfOH: trifluoromethanesulfonic acid; Pd-PEPPSI-IPent-Cl: [1,3-bis(2,6-di-3-pentylphenyl)imidazol-2-ylidene](3-chloropyridyl)dichloropalladium(II); FA: formic acid; AcOH: acetic acid; NaOAc: sodium acetate; NaBH(OAc)₃: sodium triacetoxyborohydride; PMB: p-methoxybenzyl; OTf: trifluoromethanesulfonyl.

### Example 1: 3-(1-Methyl-2-oxo-1,2-dihydro-3H-naphtho[1,2-d]imidazol-3-yl)piperidine-2,6-dione

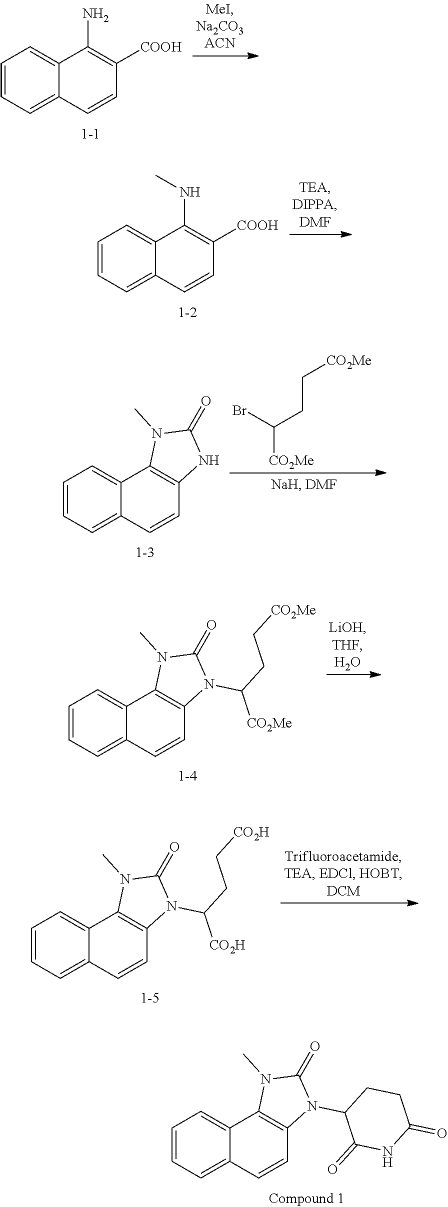

### Step 1: 1-(methylamino)-2-naphthoic acid (compound 1-2)

Compound 1-1 (0.5 g, 2.67 mmol) and sodium carbonate (424 mg, 4.01 mmol) were dissolved in acetonitrile (10 mL), followed by the addition of methyl iodide (341 mg, 2.40 mmol). The reaction mixture was stirred in an oil bath at 90° C. for 1 h. After the reaction was completed, the reaction mixture was concentrated to give a crude product, which was purified by reversed-phase column chromatography (eluent: water:acetonitrile=1:1) to give compound 1-2 (100 mg, yield: 18%). m/z (ESI): 202 [M+H]⁺.

### Step 2: 1-methyl-1,3-dihydro-2H-naphtho[1,2-d]imidazol-2-one (compound 1-3)

Triethylamine (60 mg, 0.59 mmol) was added to a solution of compound 1-2 (40 mg, 0.20 mmol) in N,N-dimethylformamide (2 mL) under a nitrogen atmosphere, and the reaction mixture was stirred at room temperature for 0.5 h. Diphenylphosphoryl azide (58 mg, 0.24 mmol) was then added and stirred in an oil bath at 60° C. for 1 h. After the reaction was completed, the reaction mixture was concentrated to give a crude product, which was purified by reversed-phase column chromatography (eluent: water:acetonitrile=1:1) to give compound 1-3 (39 mg, yield: 99%). m/z (ESI): 199 [M+H]⁺.

### Step 3: dimethyl 2-(1-methyl-2-oxo-1,2-dihydro-3H-naphtho[1,2-d]imidazol-3-yl)pentanedioate (Compound 1-4)

Sodium hydride (10 mg, 0.25 mmol) was slowly added to a solution of compound 1-3 (39 mg, 0.19 mmol) in N,N-dimethylformamide (2 mL) under a nitrogen atmosphere, and the reaction mixture was stirred at room temperature for reaction for 15 min. Dimethyl 2-bromopentanedioate (70 mg, 0.29 mmol) was then added and reacted at room temperature for 1 h. After the reaction was completed, water (0.5 mL) was added to the reaction mixture to give a crude product, which was purified by reversed-phase column chromatography (eluent: water:acetonitrile=1:1) to give compound 1-4 (60 mg, yield: 85%). m/z (ESI): 357 [M+H]⁺.

### Step 4: 2-(1-methyl-2-oxo-1,2-dihydro-3H-naphtho[1,2-d]imidazol-3-yl)pentanedioic acid (Compound 1-5)

Compound 1-4 (60 mg, 0.16 mmol) was dissolved in tetrahydrofuran (1 mL) and water (1 mL), and lithium hydroxide (12 mg, 0.50 mmol) was added. The reaction mixture was stirred at room temperature for reaction for 2 h. After the reaction was completed, the pH was adjusted to about 3 using concentrated hydrochloric acid, and lyophilization was performed to give a crude product of compound 1-5 (60 mg). m/z (ESI): 329 [M+H]⁺.

### Step 5: 3-(1-methyl-2-oxo-1,2-dihydro-3H-naphtho[1,2-d]imidazol-3-yl)piperidine-2,6-dione (Compound 1)

Compound 1-5 (60 mg, 0.18 mmol), 1-hydroxybenzotriazole (54 mg, 0.40 mmol), and triethylamine (55 mg, 0.54 mmol) were dissolved in dichloromethane (2 mL) at 0° C. under a nitrogen atmosphere, followed by the addition of trifluoroacetamide (20 mg, 0.18 mmol) and 1-(3-dimethylaminopropyl)-3-ethylcarbodiimide hydrochloride (73 mg, 0.38 mmol), and the reaction mixture was stirred at room temperature for reaction for 48 h. After the reaction was completed, the reaction mixture was concentrated to give a crude product, which was purified by reversed-phase column chromatography (eluent: water:acetonitrile=1:1) to give compound 1 (5 mg, yield: 8%). m/z (ESI): 310 [M+H]⁺. ¹H NMR (600 MHz, DMSO-d₆) δ 11.15 (s, 1H), 8.43 (d, J=8.7 Hz, 1H), 7.98 (d, J=8.2 Hz, 1H), 7.70 (d, J=8.7 Hz, 1H), 7.57 (t, J=7.7 Hz, 1H), 7.52 (d, J=8.8 Hz, 1H), 7.44 (t, J=7.5 Hz, 1H), 5.54 (dd, J=12.9, 5.4 Hz, 1H), 3.88 (s, 3H), 2.94 (ddd, J=17.5, 13.7, 5.4 Hz, 1H), 2.81 (qd, J=13.0, 4.3 Hz, 1H), 2.70-2.61 (m, 1H), 2.13-2.06 (m, 1H).

### Example 2: 6-(2-((S)-4-(4-Chlorophenyl)-2,3,9-trimethyl-6H-thieno[3,2-f][1,2,4]triazolo[4,3-a][1,4]diazepin-6-yl)acetamido)-N-((3-(2,6-dioxopiperidin-3-yl)-1-methyl-2-oxo-2,3-dihydro-1H-naphtho[1,2-d]imidazol-8-yl)methyl)hexanamide

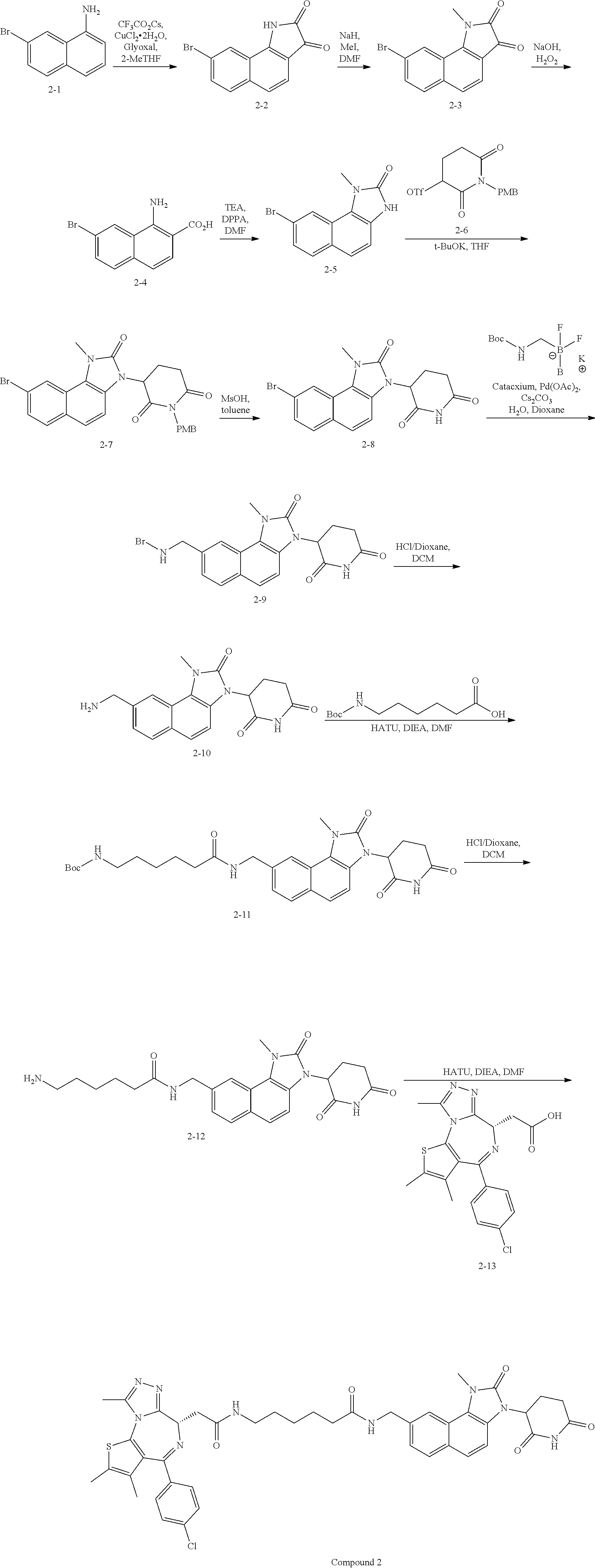

### Step 1: 8-bromo-1H-benzo[g]indole-2,3-dione (compound 2-2)

Cesium trifluoroacetate (1.1 g, 4.5 mmol) and copper(II) chloride dihydrate (3.0 g, 18 mmol) were added to a reaction mixture of compound 2-1 (1.0 g, 4.5 mmol) and glyoxal (1.7 g, 9.0 mmol) dissolved in 2-methyltetrahydrofuran (30 mL) under a nitrogen atmosphere. The reaction mixture was stirred at 70° C. for reaction for 5 h. After the reaction was completed, the reaction mixture was concentrated to give a crude product, which was purified by column chromatography (petroleum ether:ethyl acetate=1:1) to give compound 2-2 (0.6 g, yield: 48%). m/z (ESI): 277 [M+H]⁺.

### Step 2: 8-bromo-1-methyl-1H-benzo[g]indole-2,3-dione (compound 2-3)

Sodium hydride (0.1 g, 2.6 mmol) was slowly added to a reaction mixture of compound 2-2 (0.6 g, 2.1 mmol) in N,N-dimethylformamide (5 mL), and the reaction mixture was stirred at 0° C. for reaction for 0.5 h, followed by the addition of methyl iodide (0.3 g, 2.3 mmol). The reaction mixture was stirred at room temperature for reaction for 1 h. After the reaction was completed, a saturated aqueous ammonium chloride solution (10 mL) was added, and extraction was performed with ethyl acetate. The organic phase was concentrated to give a crude product of compound 2-3 (0.6 g), which was used directly in the next step. m/z (ESI): 290 [M+H]⁺.

### Step 3: 7-bromo-1-(methylamino)-2-naphthoic acid (compound 2-4)

Compound 2-3 (0.6 g, 2.0 mmol) was dissolved in an aqueous sodium hydroxide solution (1 M, 10 mL) and tetrahydrofuran (5 mL), and hydrogen peroxide (1.1 g, 10 mmol) was slowly added. The reaction mixture was reacted at room temperature for 1 h. After the reaction was completed, the pH was adjusted to about 3 with hydrochloric acid (1 M), and extraction was performed with ethyl acetate. The organic phase was concentrated to give a crude product of compound 2-4 (0.4 g), which was used directly in the next step. m/z (ESI): 280 [M+H]⁺.

### Step 4: 8-bromo-1-methyl-1,3-dihydro-2H-naphtho[1,2-d]imidazol-2-one (compound 2-5)

Triethylamine (0.43 g, 4.2 mmol) was added to a reaction mixture of compound 2-4 (0.4 g, 1.4 mmol) in N,N-dimethylformamide (5 mL) under a nitrogen atmosphere, and the reaction mixture was stirred at room temperature for 0.5 h. Diphenylphosphoryl azide (0.41 g, 1.7 mmol) was added, and the reaction mixture was stirred at room temperature for 0.5 h and then stirred at 60° C. for reaction for 1 h. After the reaction was completed, the reaction mixture was purified by reversed-phase column chromatography (eluent: water:acetonitrile=1:1) to give compound 2-5 (0.2 g, yield: 50%). m/z (ESI): 277 [M+H]⁺.

### Step 5: 3-(8-bromo-1-methyl-2-oxo-1,2-dihydro-3H-naphtho[1,2-d]imidazol-3-yl)-1-(4-methoxybenzyl)piperidine-2,6-dione (compound 2-7)

Potassium tert-butoxide (91 mg, 0.8 mmol) was added to a reaction mixture of compound 2-5 (150 mg, 0.5 mmol) in tetrahydrofuran (1 mL) under a nitrogen atmosphere, and the reaction mixture was stirred at 0° C. for reaction for 0.5 h. 1-(4-Methoxybenzyl)-2,6-dioxopiperidin-3-yl trifluoromethanesulfonate (2-6, 0.25 g, 0.65 mmol) was dissolved in tetrahydrofuran (2 mL) and slowly added dropwise to the reaction mixture, and the reaction mixture was stirred at 0° C. for reaction for 1 h. After the reaction was completed, extraction was performed with ethyl acetate, and the organic phase was concentrated to give a crude product, which was purified by reversed-phase column chromatography (eluent: water:acetonitrile=1:1) to give compound 2-7 (150 mg, yield: 54%).

m/z (ESI): 508 [M+H]⁺.

### Step 6: 3-(8-bromo-1-methyl-2-oxo-1,2-dihydro-3H-naphtho[1,2-d]imidazol-3-yl)piperidine-2,6-dione (compound 2-8)

Compound 2-7 (150 mg, 0.29 mmol) was dissolved in methanesulfonic acid (0.5 mL) and toluene (1 mL), and the reaction mixture was stirred at 120° C. for reaction for 2 h. After the reaction was completed, ice water (10 mL) was added, and extraction was performed with ethyl acetate. The organic phase was concentrated to give a crude product, which was slurried in ethyl acetate to give compound 2-8 (100 mg, yield: 87%). m/z (ESI): 388 [M+H]⁺.

### Step 7: tert-butyl ((3-(2,6-dioxopiperidin-3-yl)-1-methyl-2-oxo-2,3-dihydro-1H-naphtho[1,2-d]imidazol-8-yl)methyl)carbamate (compound 2-9)

Compound 2-8 (10 mg, 0.025 mmol), potassium (((tert-butoxycarbonyl)amino)methyl)trifluoroborate (7 mg, 0.03 mmol), palladium(II) acetate (1 mg, 0.005 mmol), di(1-adamantyl)-n-butylphosphine (2 mg, 0.007 mmol), and cesium carbonate (25 mg, 0.077 mmol) were dissolved in 1,4-dioxane (2 mL) and water (0.2 mL) under a nitrogen atmosphere, and the reaction mixture was stirred at 100° C. for reaction for 1 h. After the reaction was completed, the reaction mixture was purified by reversed-phase column chromatography (eluent: water:acetonitrile=1:1) to give compound 2-9 (10 mg, yield: 88%). m/z (ESI): 439 [M+H]⁺.

### Step 8: 3-(8-(aminomethyl)-1-methyl-2-oxo-1,2-dihydro-3H-naphtho[1,2-d]imidazol-3-yl)piperidine-2,6-dione (compound 2-10)

A solution of hydrochloric acid in dioxane (1 mL, 4 M) was added to a reaction mixture of compound 2-9 (10 mg, 0.022 mmol) in dichloromethane (2 mL), and the reaction mixture was stirred at room temperature for reaction for 5 h. After the reaction was completed, the reaction mixture was concentrated to give a crude product of compound 2-10 (8 mg), which was used directly in the next step. m/z (ESI): 339 [M+H]⁺.

### Step 9: tert-butyl (6-(((3-(2,6-dioxopiperidin-3-yl)-1-methyl-2-oxo-2,3-dihydro-1H-naphtho[1,2-d]imidazol-8-yl)methyl)amino)-6-oxohexyl)carbamate (compound 2-11)

N,N-Diisopropylethylamine (8 mg, 0.064 mmol) was added to a reaction mixture of compound 2-10 (8 mg, 0.021 mmol), 6-((tert-butoxycarbonyl)amino)hexanoic acid (5 mg, 0.022 mmol), and O-(7-azabenzotriazol-1-yl)-N,N,N′,N′-tetramethyluronium hexafluorophosphate (8 mg, 0.023 mmol) dissolved in N,N-dimethylformamide (2 mL). The reaction mixture was stirred at room temperature for reaction for 1 h. After the reaction was completed, the reaction mixture was purified by reversed-phase column chromatography (eluent: water:acetonitrile=1:1) to give compound 2-11 (7 mg, yield: 59%). m/z (ESI): 552 [M+H]⁺.

### Step 10: 6-amino-N-((3-(2,6-dioxopiperidin-3-yl)-1-methyl-2-oxo-2,3-dihydro-1H-naphtho[1,2-d]imidazol-8-yl)methyl)hexanamide (compound 2-12)

A solution of hydrochloric acid in dioxane (1 mL, 4 M) was added to a reaction mixture of compound 2-11 (7 mg, 0.012 mmol) in dichloromethane (2 mL), and the reaction mixture was stirred at room temperature for reaction for 2 h. After the reaction was completed, the reaction mixture was concentrated to give a crude product of compound 2-12 (6 mg), which was used directly in the next step. m/z (ESI): 452 [M+H]⁺.

### Step 11: 6-(2-((S)-4-(4-chlorophenyl)-2,3,9-trimethyl-6H-thieno[3,2-f][1,2,4]triazolo[4,3-a][1,4]diazepin-6-yl)acetamido)-N-((3-(2,6-dioxopiperidin-3-yl)-1-methyl-2-oxo-2,3-dihydro-1H-naphtho[1,2-d]imidazol-8-yl)methyl)hexanamide (compound 2)

Compound 2-12 (6 mg, 0.012 mmol), (S)-2-(4-(4-chlorophenyl)-2,3,9-trimethyl-6H-thieno[3,2-f][1,2,4]triazolo[4,3-a][1,4]diazepin-6-yl)acetic acid (4 mg, 0.011 mmol), and O-(7-azabenzotriazol-1-yl)-N,N,N′,N′-tetramethyluronium hexafluorophosphate (4 mg, 0.012 mmol) were dissolved in N,N-dimethylformamide (2 mL), and N,N-diisopropylethylamine (4 mg, 0.033 mmol) was added. The reaction mixture was reacted at 25° C. for 1 h. After the reaction was completed, the reaction mixture was purified by reversed-phase column chromatography (eluent: water:acetonitrile=1:1) to give compound 2 (4 mg, yield: 42%). m/z (ESI): 834 [M+H]⁺.

¹H NMR (400 MHz, DMSO-d₆) δ 11.15 (s, 1H), 8.49-8.40 (m, 2H), 8.24 (s, 1H), 8.19 (t, J=5.6 Hz, 1H), 7.92 (d, J=8.6 Hz, 1H), 7.64 (d, J=8.7 Hz, 1H), 7.51-7.47 (m, 2H), 7.41 (d, J=8.4 Hz, 2H), 7.31 (dd, J=8.5, 1.5 Hz, 1H), 5.52 (dd, J=12.7, 5.4 Hz, 1H), 4.53-4.46 (m, 3H), 3.85 (s, 3H), 3.27-3.18 (m, 3H), 3.17-3.02 (m, 2H), 2.67 (p, J=1.8 Hz, 1H), 2.59 (s, 3H), 2.40 (s, 3H), 2.33 (q, J=1.9 Hz, 1H), 2.19 (t, J=7.5 Hz, 2H), 2.12-2.05 (m, 1H), 1.61 (s, 3H), 1.57 (d, J=7.6 Hz, 2H), 1.45 (q, J=7.2 Hz, 2H), 1.33 (q, J=7.9 Hz, 2H).

### Example 3: 6-(2-((S)-4-(4-Chlorophenyl)-2,3,9-trimethyl-6H-thieno[3,2-f][1,2,4]triazolo[4,3-a][1,4]diazepin-6-yl)acetamido)-N-((3-(2,6-dioxopiperidin-3-yl)-1-methyl-2-oxo-2,3-dihydro-1H-naphtho[1,2-d]imidazol-6-yl)methyl)hexanamide

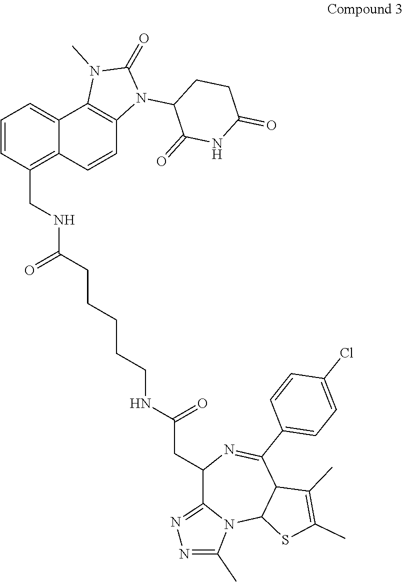 [SMILES: CC1=C(C)C2C(c3ccc(Cl)cc3)=NC(CC(=O)NCCCCCC(=O)NCc3cccc4c3ccc3c4n(C)c(=O)n3C3CCC(=O)NC3=O)c3nnc(C)n3C2S1]

Referring to the synthetic route for compound 2 the starting material compound 2-1 in the synthetic route of Example 2 was replaced with 5-bromo-1-naphthylamine to give compound 3. m/z (ESI): 834 [M+H]⁺.

¹H NMR (400 MHz, DMSO-d₆) δ 11.14 (s, 1H), 8.41-8.31 (m, 2H), 8.18 (t, J=5.7 Hz, 1H), 7.81 (d, J=9.0 Hz, 1H), 7.57-7.46 (m, 4H), 7.44-7.39 (m, 2H), 7.36 (d, J=7.1 Hz, 1H), 5.59-5.49 (m, 1H), 4.73 (d, J=5.6 Hz, 2H), 4.53-4.47 (m, 1H), 3.86 (s, 3H), 3.28-3.15 (m, 2H), 3.08 (p, J=6.9 Hz, 2H), 2.92-2.78 (m, 1H), 2.64 (d, J=16.3 Hz, 2H), 2.58 (s, 3H), 2.40 (s, 3H), 2.16 (t, J=7.4 Hz, 2H), 2.10-2.03 (m, 1H), 1.61 (s, 3H), 1.57 (t, J=7.5 Hz, 2H), 1.43 (q, J=7.3 Hz, 2H), 1.30 (q, J=8.0 Hz, 2H).

### Example 4: 3-(8-(4-((4-(((S)-2-(2-Hydroxyphenyl)-5,6,6a,7,9,10-hexahydro-8H-pyrazino[1′,2′:4,5]pyrazino[2,3-c]pyridazin-8-yl)methyl)piperidin-1-yl)methyl)piperidin-1-yl)-1-methyl-2-carbonyl-1,2-dihydro-3H-naphtho[1,2-d]imidazol-3-yl)piperidine-2,6-dione

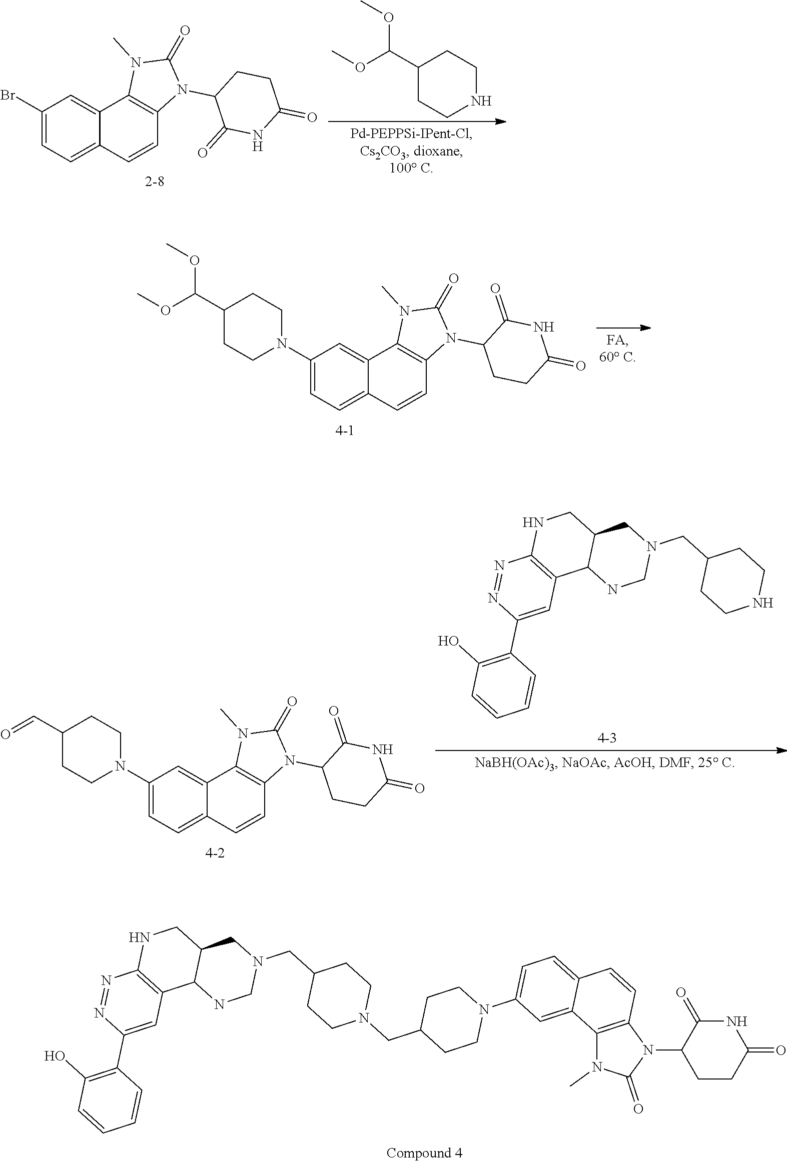

### Step 1: 3-(8-(4-(dimethoxymethyl)piperidin-1-yl)-1-methyl-2-oxo-1,2-dihydro-3H-naphtho[1,2-d]imidazol-3-yl)piperidine-2,6-dione (compound 4-1)

Compound 2-8 (160 mg, 412.14 μmol) and 4-(dimethoxymethyl)piperidine (98.44 mg, 618.21 μmol) were dissolved in dioxane (5 mL), and [1,3-bis(2,6-di-3-pentylphenyl)imidazol-2-ylidene](3-chloropyridyl)dichloropalladium(II) (17.73 mg, 20.61 μmol) and cesium carbonate (268.57 mg, 824.29 μmol) were added. The reaction mixture was stirred at 100° C. for reaction for 2 h under a nitrogen atmosphere. After the reaction was completed as detected by LCMS, the reaction mixture was diluted with water (5 mL) and extracted with ethyl acetate (20×3 mL). The organic phase was dried over anhydrous sodium sulfate and filtered, and the filtrate was concentrated under reduced pressure. The concentrate was purified by column chromatography (tetrahydrofuran:petroleum ether=1:2) to give compound 4-1 (97 mg). MS m/z (ESI): 467.3 [M+H]⁺.

### Step 2: 1-(3-(2,6-dioxopiperidin-3-yl)-1-methyl-2-oxo-2,3-dihydro-1H-naphtho[1,2-d]imidazol-8-yl)piperidine-4-carbaldehyde (compound 4-2)

Compound 4-1 (87 mg, 186.48 μmol) was dissolved in formic acid (2 mL), and the reaction mixture was stirred at 60° C. for reaction for 2 h. After the reaction was completed as detected by LCMS, the reaction mixture was concentrated under reduced pressure to give compound 4-2 (78 mg), which was used directly in the next step. MS m/z (ESI): 421.3 [M+H]⁺.

### Step 3: 3-(8-(4-((4-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)methyl)piperidin-1-yl)-1-methyl-2-carbonyl-1,2-dihydro-3H-naphtho[1,2-d]imidazol-3-yl)piperidine-2,6-dione (compound 4)

Compound 4-3 (15 mg, 35.98 μmol) and compound 4-2 (18.46 mg, 39.57 μmol) were dissolved in N,N-dimethylformamide (1 mL), and acetic acid (6.48 mg, 107.93 μmol), sodium acetate (8.85 mg, 107.93 μmol), and sodium triacetoxyborohydride (22.87 mg, 107.93 μmol) were added. The reaction mixture was stirred at 25° C. for reaction for 1 h. After the reaction was completed as detected by LCMS, the reaction mixture was quenched with water (0.1 mL) and concentrated to dryness under reduced pressure. The concentrate was purified by high performance liquid chromatography (Phenomenex Gemini NX, 5 μm silica, 30 mm diameter, 150 mm length; a mixture (1:1) of water (containing 0.05% ammonia water) and acetonitrile was used as the eluent) to give compound 4 (1.67 mg).

MS m/z (ESI): 785.5[M+H]⁺.

¹H NMR (400 MHz, DMSO-d₆) δ=14.75 (br s, 1H), 11.12 (br s, 1H), 7.91 (br d, J=8.1 Hz, 1H), 7.77 (d, J=9.2 Hz, 1H), 7.55 (s, 1H), 7.49 (d, J=8.1 Hz, 1H), 7.34 (br d, J=2.9 Hz, 1H), 7.28-7.18 (m, 4H), 6.87-6.81 (m, 2H), 5.47 (dd, J=5.0, 12.2 Hz, 1H), 4.04 (br d, J=11.0 Hz, 2H), 3.87 (s, 5H), 3.52-3.39 (m, 7H), 3.23-3.11 (m, 1H), 3.02-2.95 (m, 2H), 2.91-2.74 (m, 5H), 2.23-2.02 (m, 5H), 1.92-1.79 (m, 4H), 1.70 (br t, J=10.7 Hz, 3H), 1.26 (br d, J=10.6 Hz, 2H), 1.14 (br d, J=10.8 Hz, 2H).

### Example 5: 1-(3-Chloro-4-methylphenyl)-3-((3-(2,6-dioxopiperidin-3-yl)-1-methyl-2-oxo-2,3-dihydro-1H-naphtho[1,2-d]imidazol-8-yl)methyl)urea

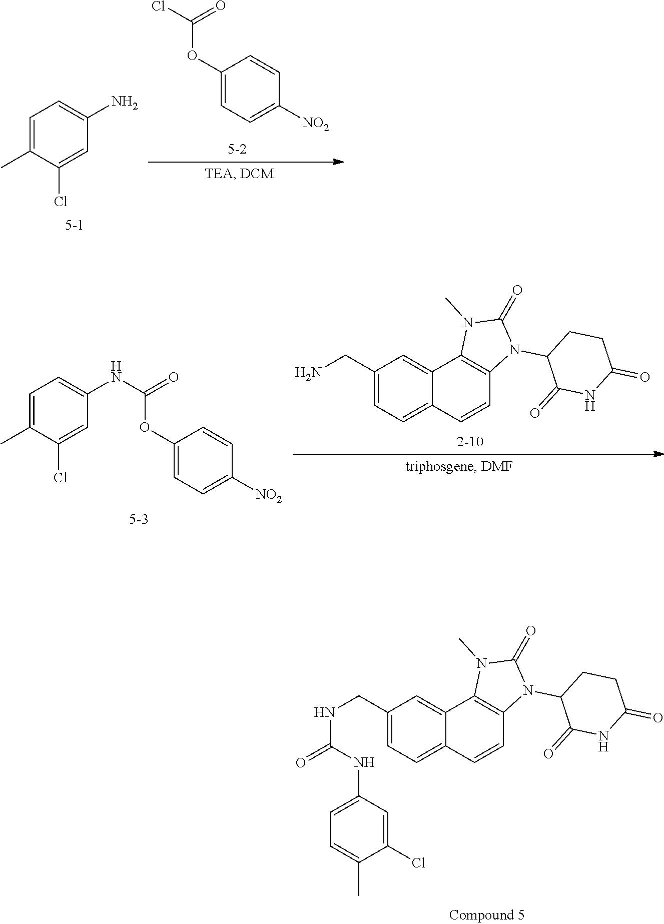

### Step 1: 4-nitrophenyl (3-chloro-4-methylphenyl)carbamate (compound 5-3)

Compound 5-1 (100 mg, 0.71 mmol) was dissolved in dichloromethane (3 mL), triethylamine (215 mg, 2.13 mmol) was added, and compound 5-2 (171 mg, 0.85 mmol) was added in an ice bath. The reaction mixture was warmed to room temperature and stirred for 1 h. After the reaction was completed, the reaction mixture was concentrated and extracted with ethyl acetate and water. The organic phase was concentrated to give a crude product of compound 5-3 (134 mg, yield: 62%). m/z (ESI): 308 [M+H]⁺.

### Step 2: 1-(3-chloro-4-methylphenyl)-3-((3-(2,6-dioxopiperidin-3-yl)-1-methyl-2-oxo-2,3-dihydro-1H-naphtho[1,2-d]imidazol-8-yl)methyl)urea (compound 5)

Compound 2-10 (132 mg, 0.39 mmol), triphosgene (97 mg, 0.96 mmol), and N,N-dimethylformamide (1 mL) were added to a flask, and the system was vacuumized and purged with nitrogen. A solution of compound 5-3 (100 mg, 0.32 mmol) in N,N-dimethylformamide was added to the flask in an ice bath, and after the addition, the reaction mixture was stirred at room temperature for 1 h. After the reaction was completed, the reaction mixture was cooled by stirring in an ice bath at 0° C., quenched with water slowly, and extracted with ethyl acetate and water. The organic phase reaction mixture was concentrated to give a crude product, which was purified by reversed-phase column chromatography (eluent: water:acetonitrile=1:1) to give compound 5 (142 mg, yield: 88%). m/z (ESI): 506[M+H]⁺.

¹H NMR (400 MHz, DMSO-d₆) δ 11.13 (s, 1H), 8.83 (s, 1H), 8.30 (s, 1H), 7.94 (d, J=8.6 Hz, 1H), 7.72-7.66 (m, 2H), 7.46 (d, J=8.8 Hz, 1H), 7.38 (d, J=8.5 Hz, 1H), 7.25-7.15 (m, 2H), 6.89 (t, J=6.0 Hz, 1H), 5.52 (dd, J=12.6, 5.3 Hz, 1H), 4.51 (d, J=5.9 Hz, 2H), 3.86 (s, 3H), 3.00-2.73 (m, 2H), 2.69-2.65 (m, 1H), 2.23 (s, 3H), 2.11-2.05 (m, 1H).

### Example 6: 1-(3-Chloro-4-methylphenyl)-3-((3-(2,6-dioxopiperidin-3-yl)-1-methyl-2-oxo-2,3-dihydro-1H-naphtho[1,2-d]imidazol-7-yl)methyl)urea

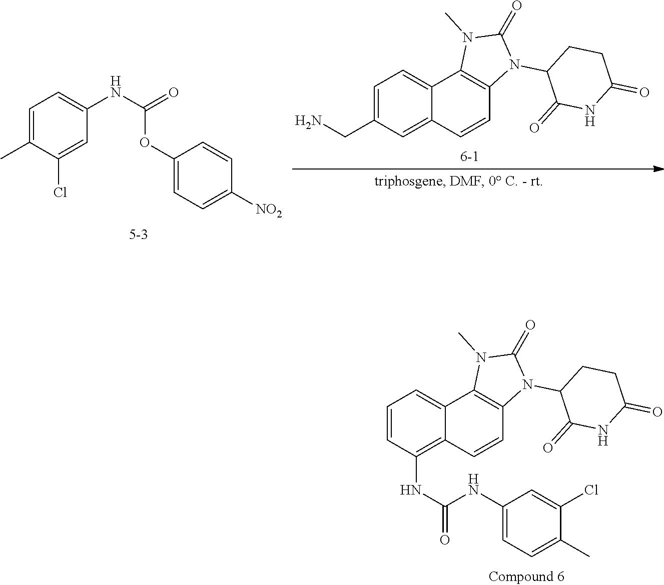 [SMILES: Cc1ccc(NC(=O)Oc2ccc([N+](=O)[O-])cc2)cc1Cl ; Cn1c(=O)n(C2CCC(=O)NC2=O)c2ccc3cc(CN)ccc3c21 ; Cc1ccc(NC(=O)Nc2cccc3c2ccc2c3n(C)c(=O)n2C2CCC(=O)NC2=O)cc1Cl]

Step 1: 3-(7-(aminomethyl)-1-methyl-2-carbonyl-1,2-dihydro-3H-naphtho[1,2-d]imidazol-3-yl)piperidine-2,6-dione (compound 6-1) Referring to the synthetic route for compound 2-10, the starting material compound 2-1 in the synthetic route of Example 2 was replaced with 6-bromo-1-naphthylamine to give compound 6-1. m/z (ESI): 339 [M+H]⁺.

### Step 2: 1-(3-chloro-4-methylphenyl)-3-((3-(2,6-dioxopiperidin-3-yl)-1-methyl-2-oxo-2,3-dihydro-1H-naphtho[1,2-d]imidazol-7-yl)methyl)urea (compound 6)

Referring to the last step in the synthetic route of Example 5, compound 2-10 was replaced with compound 6-1, and synthesis was performed to give compound 6 (128 mg, yield: 79%) m/z (ESI): 506[M+H]⁺.

¹H NMR (400 MHz, DMSO-d₆) δ 11.12 (s, 1H), 8.72 (s, 1H), 8.39 (d, J=8.9 Hz, 1H), 7.83 (s, 1H), 7.70-7.62 (m, 2H), 7.55-7.46 (m, 2H), 7.22-7.13 (m, 2H), 6.78 (t, J=6.0 Hz, 1H), 5.51 (dd, J=12.6, 5.4 Hz, 1H), 4.45 (d, J=6.0 Hz, 2H), 3.85 (s, 3H), 2.91 (s, 1H), 2.86-2.72 (m, 1H), 2.67 (s, 1H), 2.23 (s, 3H), 2.12-2.06 (m, 1H).

### Example 7: 1-(3-Chloro-4-methylphenyl)-3-((3-(2,6-dioxopiperidin-3-yl)-1-methyl-2-oxo-2,3-dihydro-1H-naphtho[1,2-d]imidazol-6-yl)methyl)urea

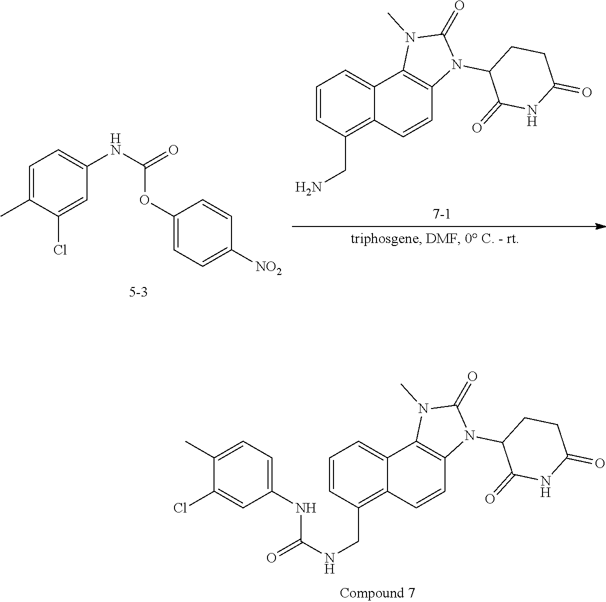 [SMILES: Cc1ccc(NC(=O)Oc2ccc([N+](=O)[O-])cc2)cc1Cl ; Cn1c(=O)n(C2CCC(=O)NC2=O)c2ccc3c(CN)cccc3c21 ; Cc1ccc(NC(=O)NCc2cccc3c2ccc2c3n(C)c(=O)n2C2CCC(=O)NC2=O)cc1Cl]

### Step 1: 3-(6-(aminomethyl)-1-methyl-2-carbonyl-1,2-dihydro-3H-naphtho[1,2-d]imidazol-3-yl)piperidine-2,6-dione (compound 7-1)

Referring to the synthetic route for compound 2-10, the starting material compound 2-1 in the synthetic route of Example 2 was replaced with 5-bromo-1-naphthylamine to give compound 7-1. m/z (ESI): 339 [M+H]⁺.

### Step 2: 1-(3-chloro-4-methylphenyl)-3-((3-(2,6-dioxopiperidin-3-yl)-1-methyl-2-oxo-2,3-dihydro-1H-naphtho[1,2-d]imidazol-6-yl)methyl)urea (compound 7)

Referring to the last step in the synthetic route of Example 5, compound 2-10 was replaced with compound 7-1, and synthesis was performed to give compound 7 (128 mg, yield: 79%). m/z (ESI): 506[M+H]⁺.

¹H NMR (400 MHz, DMSO-d₆) δ 11.12 (s, 1H), 8.79 (s, 1H), 8.39 (d, J=8.7 Hz, 1H), 7.89 (d, J=9.0 Hz, 1H), 7.68 (d, J=2.2 Hz, 1H), 7.63-7.50 (m, 2H), 7.43 (d, J=7.0 Hz, 1H), 7.17 (d, J=8.4 Hz, 1H), 7.12 (dd, J=8.3, 2.2 Hz, 1H), 6.85 (t, J=5.8 Hz, 1H), 5.54 (dd, J=12.7, 5.4 Hz, 1H), 4.76 (d, J=5.9 Hz, 2H), 3.87 (s, 3H), 3.01-2.74 (m, 2H), 2.71-2.62 (m, 1H), 2.23 (s, 3H), 2.14-2.03 (m, 1H).

### Comparative Example 1: 3-(1-Methyl-2-oxo-1,2,6,7,8,9-hexahydro-3H-imidazo[4,5-h]isoguinolin-3-yl)piperidine-2,6-dione

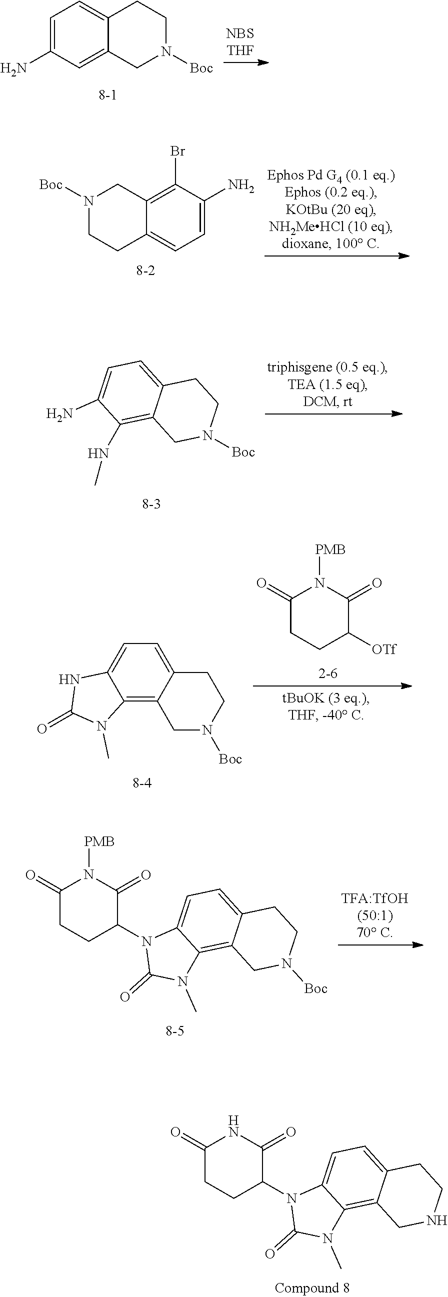

### Step 1: tert-butyl 7-amino-8-bromo-3,4-dihydroisoquinoline-2(1H)-carboxylate (compound 8-2)

Compound 8-1 (1 g, 4 mmol) was dissolved in tetrahydrofuran (5 mL), and N-bromosuccinimide (778 mg, 4.4 mmol) was added in batches in an ice bath. The reaction mixture was stirred at room temperature for 1 h. After the reaction was completed, the reaction mixture was concentrated and extracted with ethyl acetate and water. The organic phase was concentrated to give a crude product, which was purified by reversed-phase column chromatography (eluent: water:acetonitrile=1:1) to give compound 8-2 (1.22 g, yield: 91%). m/z (ESI): 327 [M+H]⁺.

### Step 2: tert-butyl 7-amino-8-(methylamino)-3,4-dihydroisoquinoline-2(1H)-carboxylate (Compound 8-3)

Compound 8-2 (1 g, 3.05 mmol), methanesulfonato{dicyclohexyl[3-(1-methylethoxy)-2′,4′,6′-tris(1-methylethyl)-1,1′-biphenyl-2-yl]phosphine}(2′-methylamino-1,1′-biphenyl-2-yl)palladium(II) (280 mg, 0.305 mmol), dicyclohexyl(3-isopropoxy-2′,4′,6′-triisopropyl-[1,1′-biphenyl]-2-yl)phosphane (325 mg, 0.610 mmol), potassium tert-butoxide (6.83 g, 61 mmol), and methylamine hydrochloride (2.05 g, 30.5 mmol) were placed in a flask, and the system was vacuumized and purged with nitrogen. 1,4-Dioxane (100 mL) was then added, and the reaction mixture was stirred in an oil bath at 100° C. for 5 h. After the reaction was completed, the reaction mixture was cooled by stirring in an ice bath at 0° C., quenched with water slowly, and extracted with ethyl acetate and water. The organic phase reaction mixture was concentrated to give a crude product, which was purified by reversed-phase column chromatography (eluent: water:acetonitrile=1:1) to give compound 8-3 (524 mg, yield: 61%). m/z (ESI): 278 [M+H]⁺.

### Step 3: tert-butyl 1-methyl-2-oxo-1,2,3,6,7,9-hexahydro-8H-imidazo[4,5-h]isoquinoline-8-carboxylate (compound 8-4)

Compound 8-3 (500 mg, 1.79 mmol) and triethylamine (268 mg, 2.68 mmol) were dissolved in dichloromethane (5 mL). Triphosgene (264 mg, 0.89 mmol) was dissolved in dichloromethane (2 mL), and the solution was slowly added to the reaction mixture in an ice bath. The reaction mixture was stirred at room temperature for 1 h. After the reaction was completed, the reaction mixture was concentrated and extracted with ethyl acetate and water. The organic phase was concentrated to give a crude product, which was purified by reversed-phase column chromatography (eluent: water:acetonitrile=1:1) to give compound 8-4 (293 mg, yield: 54%). m/z (ESI): 304 [M+H]⁺.

### Step 4: tert-butyl 3-(1-(4-methoxybenzyl)-2,6-dioxopiperidin-3-yl)-1-methyl-2-oxo-1,2,3,6,7,9-hexahydro-8H-imidazo[4,5-h]isoquinoline-8-carboxylate (compound 8-5)

Compound 8-4 (50 mg, 0.16 mmol) and potassium tert-butoxide (53 mg, 0.48 mmol) were dissolved in tetrahydrofuran (2 mL) and stirred for 10 min in a dry ice bath. Compound 2-6 (91 mg, 0.24 mmol) was dissolved in tetrahydrofuran (1 mL), and the solution was slowly added to the reaction mixture and stirred for 1 h. After the reaction was completed, the reaction was quenched with ice water, and extraction was performed with ethyl acetate and water. The organic phase was concentrated to give a crude product, which was purified by reversed-phase column chromatography (eluent: water:acetonitrile=1:1) to give compound 8-5 (51 mg, yield: 60%). m/z (ESI): 535 [M+H]⁺.

### Step 5: 3-(1-methyl-2-oxo-1,2,6,7,8,9-hexahydro-3H-imidazo[4,5-h]isoquinolin-3-yl)piperidine-2,6-dione (compound)

Compound 8-5 (50 mg, 0.093 mmol) was dissolved in trifluoroacetic acid (2 mL), and trifluoromethanesulfonic acid (0.04 mL) was added. The reaction mixture was stirred at 70° C. for 2 h. After the reaction was completed, the reaction mixture was purified by reversed-phase column chromatography (eluent: water:acetonitrile=1:1) to give compound 8 (18 mg, yield: 60%). m/z (ESI): 315 [M+H]⁺.

¹H NMR (400 MHz, DMSO-d₆) δ 11.08 (s, 1H), 7.08 (d, J=8.2 Hz, 1H), 6.92 (d, J=8.2 Hz, 1H), 5.36 (dd, J=12.6, 5.4 Hz, 1H), 4.77 (t, J=4.7 Hz, 2H), 3.56 (s, 4H), 3.38-3.31 (m, 2H), 3.03 (t, J=6.2 Hz, 2H), 2.95-2.84 (m, 1H), 2.72 (td, J=12.9, 4.3 Hz, 1H), 2.63 (dt, J=16.4, 2.8 Hz, 1H), 2.04-1.96 (m, 1H).

### Test Examples for Biological Activity and Related Properties

### Test Example 1: Cereblon Binding Experiment

### 1. Experimental Instruments and Materials

| Instrument | Manufacturer | Model |
|---|---|---|
| Oscillator | Boxun | BSD-YX3400 |
| Plate reader | PerkinElmer | Envision |
| Centrifuge | Eppendorf | Microplate Shaker, |
| | | Catalog#5353 |
| Compound dilution and | PerkinElmer | Echo |
| loading instrument | | |

The detection kit (HTRF Human Cereblon Binding Kits) used in the experiment was a detection method for the quantitative measurement of Cereblon WT ligands using HTRF® technology. The detection principle was based on HTRF technology, where the specifically labeled GST antibody (Euroum Cryptate, donor) simultaneously bound to the GST-labeled human Cereblon WT ligand and the XL665-labeled lenalidomide tracer (acceptor). The donor was excited by a light source to induce fluorescence resonance energy transfer (FRET) to the acceptor, and the acceptor emitted fluorescence at a specific wavelength of 665 nm. After the addition of a compound, the compound competed with the XL665-labeled lenalidomide to prevent FRET from occurring. The FRET signal ratio was inversely proportional to the concentration of the compound.

The information on the additional reagent and consumable required for the experiment was as follows:

| Reagent | Brand | Catalog No. |
|---|---|---|
| HTRF Human Cereblon Binding Kits | Promega | 64BDCRBNPEG |
| 384-well plate | PerkinElmer | 6007299 |
| Lenalidomide | Accela | SY047646 |

### 2. Experimental Procedures

The compounds of the present disclosure were dissolved in DMSO at a stock concentration of 10 mM. The dose-response program of the compound dilution and loading instrument (Echo) was used to perform gradient dilution of the compound stock solution. The total experimental volume of the dilution program was 20 μL, with an initial concentration of 100 μM for the compound to be tested and 200 μM for the standard. A 4-fold dilution was performed, resulting in 8 concentration points, and the DMSO content was 1%. After the program was completed, 5 μL of the 1×9 #diluent from the kit was added to each well, followed by the addition of 5 μL of the GST-labeled human Cereblon WT ligand. After thorough mixing, 10 μL of the HTRF detection reagent was added, and the mixture was incubated at room temperature for 3 h. The HTRF signal in each well was measured using an Envision plate reader. 100% binding inhibition was defined as the signal ratio under treatment with 200 μM standard lenalidomide.

### 3. Data Analysis

The ratio of acceptor to donor emission signals was calculated for each well:

$$Ratio = signal at 665 nm / signal at 620 nm$$

$$Coefficient of deviation ( \% ) = standard deviation / average ratio$$

$$Cereblon binding inhibition ratio ( \% ) = 100 \% - 100 \% \times ( Sample - L ) / ( H - L )$$

wherein:
Sample=Ave (test sample group);
H=Ave (DMSO-treated group);
L=Ave (200 μM lenalidomide standard-treated group).

Data analysis was performed by GraphPad Prism 9. The concentration-response curves were obtained using non-linear four-parameter fitting, and the IC₅₀ for the compound was calculated:

$$Y = Bottom + ( Top - Bottom ) / ( 1 + 10 \hat{} ( ( Log {IC}_{50} - X ) \times HillSlope ) )$$

where:
X: Log of the compound concentration;
Y: inhibition rate (%);
Bottom is the minimum percent inhibition;
Top is the maximum percent inhibition; and
HillSlope is the slope factor of the curve.

The binding ability of the compounds of the present disclosure to Cereblon was determined through the above test, and the measured IC₅₀ values were shown in Table 1.

TABLE 1

| Compound No. | IC₅₀ (μM) |
|---|---|
| Compound 1 | 0.188 |
| Compound 8 | 8.7 |

### Test Example 2: Experiment for Antiproliferative Activity of MV-4-11 Cells

### 1. Experimental Instruments and Materials

### Instrument and Device

| Instrument | Model | Manufacturer |
|---|---|---|
| Centrifuge | 5800 | Eppendorf |
| Cell counter | IC1000 | Count star |
| Inverted microscope | TS2 | Nikon |
| Cell incubator | 1IVIOS 250i | Thermo |
| Envision microplate reader | Envision2105 | Perkin Elmer |

### Experimental Reagent and Consumable

| Name | Catalog No. | Brand |
|---|---|---|
| IMDM medium | 12440-053 | Gibco |
| FBS | 10099-141C | Gibco |
| Trypsin-EDTA (0.25%), phenol red | 25200056 | Gibco |
| PBS | SH30256.01 | Cytiva |
| CellTiter-Glo ® Cell Viability Assay | G9241 | Promega |
| 96-well black/clear round bottom ultralow- | 4515 | Corning |
| adsorption plate | | |
| dBET6 | B880855 | Macklin |

### 2. Experimental Procedures

### (1) Cell Plating

The medium of the target cells MV-4-11 (CBP60522, Cobioer) was removed, and PBS was added for rinsing once, followed by digestion with trypsin (Trypsin-EDTA (0.25%)) for 5 min. After the digestion, 10 mL of a complete medium (IMDM+10% FBS) was added to neutralize the trypsin. The cells were then pipetted and collected, centrifuged at 1000 rpm for 5 min, and counted. The cell density was adjusted to 30,000 cells/mL. 90 μL of the cell suspension was added to a 96-well low-adsorption plate. 200 μL of PBS was added to the edge wells of the 96-well plate. The plate was centrifuged at 1000 rpm for 5 min to form cell spheroids, which were then incubated in a cell incubator overnight.

### (2) Drug Addition to Cells

The compounds of the present disclosure were dissolved in DMSO at a stock concentration of 10 mM. Before drug administration, the compound was subjected to a 5-fold gradient dilution using DMSO, resulting in 8 gradient working solutions. 2 μL of working solutions at different concentrations were each added to a dilution plate containing 198 μL of a medium, and the mixtures were mixed thoroughly by pipetting. 10 μL of the medium containing the compound was then transferred from the dilution plate to the cell plate in which 90 μL of the cell suspension had been plated the day before. The final concentrations of the compound at each gradient were 1000 nM, 200 nM, 40 nM, 8 nM, 1.6 nM, 0.32 nM, 0.064 nM, and 0.0128 nM. The positive control compound was dBET6. The diluted compound was added at 10 μL/well, and the cell plate was incubated at 37° C. with 5% CO₂ for 3 days.

### (3) CellTiter-Glo® Cell Viability Assay Detection

The cells were removed from the incubator and allowed to return to room temperature within 30 min. 50 μL of the CellTiter-Glo® Cell Viability Assay reagent was added, and the plate was shaken to mix thoroughly for 10 min before it was read on the Envision microplate reader.

### 3. Data Analysis

The antiproliferative activity of the compounds of the present disclosure for MV-4-11 cells was determined through the above test, and the cell growth inhibition rate for each sample well was calculated based on the raw data.

$$Inhibition rate ( \% ) = 100 \% \times ( 1 - sample reading / reference average reading )$$

Data analysis was performed by GraphPad Prism 9. The concentration-response curves were obtained using non-linear four-parameter fitting, and the IC₅₀ for the compound was calculated:

$$Y = Bottom + ( Top - Bottom ) / ( 1 + 10 \hat{} ( ( Log {IC}_{50} - X ) \times HillSlope ) )$$

wherein:
X: Log of the compound concentration;
Y: inhibition rate (%);
Bottom is the minimum percent inhibition;
Top is the maximum percent inhibition; and
HillSlope is the slope factor of the curve.

The antiproliferative activity of the compounds of the present disclosure for MV-4-11 cells is shown in Table 2.

TABLE 2
Antiproliferative activity of compounds of
the present disclosure for MV-4-11 cells

| Compound No. | IC₅₀ (nM) |
|---|---|
| Compound 2 | 3.80 |
| Compound 3 | 14.14 |

### Test Example 3: Experiment for Degradation Activity for BRD4 Proteins in MV-4-11 Cells

### 1. Experimental Instruments and Materials

### Instrument and Device

| Instrument | Manufacturer | Model |
|---|---|---|
| Centrifuge | Eppendorf | 5800 |
| Cell counter | Count star | IC1000 |
| Inverted microscope | Nikon | TS2 |
| Cell incubator | Thermo | 1IVIOS 250i |
| Electrophoresis apparatus | Bio-Rad | 1645050 |
| Transfer apparatus | Bio-Rad | Trans-Blot Turbo |
| | | (1704150) |
| Azure WB imaging system | Azure biosystems | Sapphire |

### Experimental Reagent and Consumable

| Name | Catalog No. | Brand |
|---|---|---|
| IMDM medium | 12440-053 | Gibco |
| FBS | 10099-141C | Gibco |
| Trypsin-EDTA (0.25%), phenol red | 25200056 | Gibco |
| PBS | SH30256.01 | Cytiva |
| β-actin antibody | ab8227 | Abcam |
| BRD4(E2A7X) Rabbit mAb | 13440S | Cell Signaling |
| | | Technology |
| Goat Anti-Rabbit IgG H&L (HRP) | 500ug/ | Abcam |
| | ab205718 | |
| RIPA lysis buffer | 89900 | Thermo Fisher |
| 4-15% Mini-PROTEAN ® TGX ™ | 4561086 | Bio-Rad |
| Precast Protein Gels, 15-well, 15 μL | | |
| Pierce BCA protein detection kit | 23227 | Thermo Fisher |
| Bovine Serum Albumin (BSA) | V900933-100G | Sigma |
| Tris-buffered saline with 0.1% | C520009-0001 | Sangon Biotech |
| Tween ® 20 Detergent (TBST) | | |
| SuperSignal West Atto | A38554r | Thermo Fishe |
| 5 × SDS-PAGE protein loading buffer | C508320-0010 | Sangon Biotech |
| 12-well plate | 3512 | Corning |

### 2. Experimental Procedures

### (1) Cell Plating

The medium of MV-4-11 (CBP60522, Cobioer) was removed, and PBS was added for rinsing once, followed by digestion with trypsin (Trypsin-EDTA (0.25%)) for 5 min. After the digestion, 10 mL of a complete medium (an IMDM medium containing 10% FBS) was added to neutralize the trypsin. The cells were then pipetted and collected, centrifuged at 1000 rpm for 5 min, and counted. The cell density was adjusted to 500,000 cells/mL. 2 mL of the cell suspension was added to a 12-well plate, and the plate was incubated in a cell incubator overnight.

### (2) Drug Addition to Cells

The compounds of the present disclosure were dissolved in DMSO at a stock concentration of 10 mM. The compound stock solution was subjected to gradient dilution (4-fold dilution), resulting in 8 concentration points. DMSO was used as the negative control group, and the DMSO content was 0.1%. After mixing the compound with the cell suspension thoroughly, the mixture was incubated at 37° C. with 5% CO₂ for 6 h.

### (3) Western Blot

Six hours after drug treatment, the supernatant was discarded, and the cells were washed with 1000 μL of pre-cooled PBS. 100 μL of RIPA lysis buffer (25 mM Tris-HCl, pH=7.5, 150 mM NaCl, 1% NP-40, 1 mM EDTA, pH=8.0, 1 mM PMSF, 1 mM Na₃VO₄, and 1× Protease Inhibitor Cocktail-P2714, Sigma) was added and placed on ice for lysis for 10 min. The lysed cell samples were collected and centrifuged at 12000 rpm at 4° C. for 30 min. The concentration of each protein sample was determined using the Pierce BCA kit for protein quantification. An appropriate amount of 5×SDS-PAGE protein loading buffer was added to the collected protein samples, and the mixtures were then heated in a metal bath at 100° C. for 10 min to fully denature the proteins. The protein samples were loaded into the SDS-PAGE gel loading wells and electrophoresed at a constant voltage of 120 V for 60 min. After the electrophoresis, the gel was transferred to a PVDF membrane using a Bio-bad transfer apparatus. After the transfer, the band was cut according to the size of the target protein and blocked with 5% BSA (prepared by dissolving 5 g of BSA in 100 mL of TBST) at room temperature for 1 h. Subsequently, BRD4 (1:3000) and -actin (1:3000) antibodies were added and incubated at 4° C. overnight. The next day, the antibodies were recovered, and the band was washed 3 times with PBST (PBS containing 0.1% Tween-20) for 10 min each time. After washing, the diluted Goat Anti-Rabbit IgG H&L (Goat Anti-Rabbit secondary antibody) (1:3000) was added and incubated at room temperature for 1 h. The band was washed 3 times with PBST (PBS containing 0.10% Tween-20). The proteins were detected using the SuperSignal West Atto ultra-sensitive ECL luminescent liquid.

### 3. Data Analysis

The degradation activity for BRD4 proteins in MV-4-11 cells by the compounds of the present disclosure was determined through the above test. The gray values of the target sample bands were read using Image J software, and the protein degradation rate for each sample well was calculated based on the raw data.

$$Degradation rate ( \% ) = 100 \% \times ( 1 - sample reading / DMSO reference reading )$$

wherein:
All raw data were processed for data analysis using GraphPad Prism 9. The concentration-response curves were obtained using non-linear four-parameter fitting, and the DC₅₀ for the compound was calculated:

$$Y = Bottom + ( Top - Bottom ) / ( 1 + 10 \hat{} ( ( Log {IC}_{50} - X ) \times HillSlope ) )$$

where: X: Log of the compound concentration;
Y: degradation rate (%);
Bottom is the minimum percent degradation;
Top is the maximum percent degradation; and
HillSlope is the slope factor of the curve.

The measured DC₅₀ values for the compounds of the present disclosure are shown in Table 3.

TABLE 3
BRD4 protein degradation activity of
compounds of the present disclosure

| Compound No. | Maximum degradation rate (%) | DC₅₀ (nM) |
|---|---|---|
| Compound 2 | 85.60 | 2.19 |
| Compound 3 | 74.34 | 11.19 |

### Test Example 4: Detection of BRM and BRG1 Protein Degradation Activity of Compound Using HiBiT Detection Technology

### 1. Cell Line Construction:

The effect of the compound on the degradation of BRM and BRG1 proteins was examined using the HiBiT detection technology. The HiBiT tag was inserted after the start codon or before the stop codon of BRM and BRG1 proteins. When the compound degraded the BRM or BRG1 proteins, the degradation of the BRM and BRG1 proteins can be quantified by measuring the expression level of the HiBiT tag. Based on the insertion site of HiBiT, sgRNA and donor DNA for BRM and BRG1 were designed. A Cas9 transfection reagent (TrueCut™ Cas9 Protein v2, A36498, Invitrogen; Lipofectamine™ CRISPRMAX™ Cas9 transfection reagent, CMAX00003, Invitrogen; Opti-MEM™ I Reduced Serum Medium, 31985070, Thermofisher) was prepared. According to the Cas9 transfection reagent instruction, the required amounts of various reagents, sgRNA, and donor DNA were calculated. According to the Cas9 transfection protocol, transfection was performed on SW1573 cells, and after 24 h, the cells were cultured in a normal medium until 3 days after transfection. Cells from each group were expanded, and the expression level of the HiBiT tag in the pool was detected using the Nano Glo HiBiT Lytic Detection System (N3050, Promega). The cell pool with a high HiBiT tag expression level was seeded in a 96-well plate in a monoclonal form for expansion, and the expression level of the HiBiT tag in monoclones was detected using the Nano Glo HiBiT Lytic Detection System (N3050, Promega). If the detected HiBiT tag fluorescence value from the expanded monoclonal cells was very high, it indicated that the HiBiT tag might have been successfully connected to the group of cells. These monoclonal cells were expanded and sequenced to confirm that the HiBiT sequence was successfully connected and was homozygous, making them suitable for the subsequent degradation experiment.

### 2. Cell Seeding and Compound Administration:

SW1573 HiBiT knock-in cells constructed using the method described above were digested and pipetted in a cell culture flask and resuspended in a medium (a DMEM medium (Thermo Fisher, Catalog No. 11995073) containing 10% FBS (Thermo Fisher, Catalog No. 10099141C) and 1% Penicillin-Streptomycin Solution (Thermo Fisher, Catalog No. 15140-122)). The cell concentration (SW1573 HiBiT knock-in cells, 9000 cells/well) was adjusted, and the cells were seeded in a 384-well plate, which was then incubated at 37° C. with 5% CO₂ overnight. The compound to be tested was formulated into a 10 mM stock solution with DMSO, and a small amount of the stock solution was diluted to 1 mM with DMSO for later use. A 1 mM compound solution and DMSO were prepared. Using the Echo 650 Series Acoustic Liquid Handlers (Beckman, model: Echo 650), the 1 mM compound solution to be tested was subjected to a 3-fold gradient dilution, resulting in 10 concentration points. 30 nL of the compound was transferred to a 384-well cell culture plate containing the medium and subjected to a 1000-fold dilution. The resulting final concentrations of the compound were 1000 nM, 333.33 nM, 111.11 nM, 37.04 nM, 12.35 nM, 4.12 nM, 1.37 nM, 0.46 nM, 0.15 nM, and 0.05 nM, respectively. The 384-well cell culture plate with the added compound was then incubated at 37° C. with 5% CO₂ for another 16-18 h.

### 3. Protein Degradation Detection:

15 μL of the reaction mixture (Nano Glo HiBiT Lytic Detection System (N3050, Promega) was prepared according to the protocol) was added to each well of the 384-well plate, and the plate was incubated at room temperature for 10 min and scanned and analyzed using the Envision multifunctional microplate reader (purchased from PerkinElmer).

### 4. Calculation of Degradation DC₅₀

The degradation rate of proteins at each concentration was calculated using EXCEL XLfit5.4.0, with 2 replicates for each concentration point. Protein degradation curves were generated, and DC₅₀ and Dₘₐₓ were calculated.

$$Protein degradation rate ( \% ) = ( High control - corresponding well reading ) / ( High control - Low control ) \times 100 \%$$

High control=0.1% DMSO, defined as 0% degradation
Low control=PBS (cell-free), defined as 100% degradation

A degradation curve was generated from the degradation rates at 10 concentration points, and the DC₅₀ and Dₘₐₓ values were calculated.

TABLE 4

| | Degradation of BRM | | Degradation of BRG1 | |
|---|---|---|---|---|
| | (SW1573 cells) | | (SW1573 cells) | |
| Compound | DC₅₀ (nM) | Dₘₐₓ (%) | DC₅₀ (nM) | Dₘₐₓ (%) |
| 4 | 0.27 | 96 | 3.77 | 73 |

The test results showed a compound as the activity of degrading BRM and RG1 of SW1573 cells.

1. A compound represented by formula (I) or a pharmaceutically acceptable salt thereof: 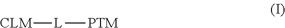
wherein,
CLM has the structure shown below: 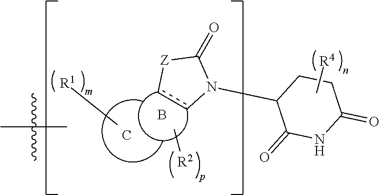
“ [symbol] ” is selected from a single bond and a double bond;
Z is selected from C(R³)₂, NR³, and O;
ring B is selected from 5- to 6-membered heteroaromatic ring, 5- to 8-membered heterocyclic ring, benzene ring, and C₅-C₈ saturated or partially saturated carbon ring;
ring C is selected from 5- to 6-membered heteroaromatic ring, 5- to 8-membered heterocyclic ring, benzene ring, and C₅-C₈ saturated or partially saturated carbon ring;
R¹, R², and R⁵ are each independently selected from halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
each R³ is independently selected from H, halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
or R¹ and R³, together with the atoms linked thereto, form C₅-C₈ saturated or partially saturated carbon ring, 5- to 6-membered heteroaromatic ring, or 5- to 8-membered heterocyclic ring, wherein the C₅-C₈ saturated or partially saturated carbon ring, 5- to 6-membered heteroaromatic ring, or 5- to 8-membered heterocyclic ring is optionally substituted with R⁵;
each R⁴ is independently selected from halogen, CN, NO₂, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₂-C₁₀ alkenyl, C₂-C₁₀ alkynyl, C₃-C₁₀ cycloalkyl, and 4- to 8-membered heterocyclyl, wherein the 2- to 10-membered heteroalkyl, C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and p are independently selected from 0, 1, 2, 3, 4, 5, and 6;
L represents a linking unit of CLM and PTM;
PTM is selected from binding moieties of targeted proteins.

2. The compound represented by formula (I) or the pharmaceutically acceptable salt thereof according to claim 1, wherein Z is selected from NR³ and O; or
wherein ring B is selected from 5- to 6-membered heteroaromatic ring, 5- to 6-membered heterocyclic ring, benzene ring, and C₅-C₆ saturated or partially saturated carbon ring; or
wherein ring C is selected from 5- to 6-membered heteroaromatic ring, 5- to 6-membered heterocyclic ring, benzene ring, and C₅-C₆ saturated or Partially saturated carbon ring.

3. (canceled)

4. (canceled)

5. The compound represented by formula (I) or the pharmaceutically acceptable salt thereof according to claim 1, wherein CLM has a structure represented by formula (II): 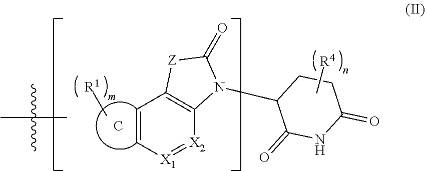
wherein X₁ and X₂ are independently selected from N and CH, wherein the CH is optionally substituted with R²; ring C, Z, R¹, R², R⁴, m, and n are as defined in claim 1.

6. The compound represented by formula (I) or the pharmaceutically acceptable salt thereof according to claim 1, wherein
CLM is selected from structures represented by formulas (II-1a) and (II-1b): 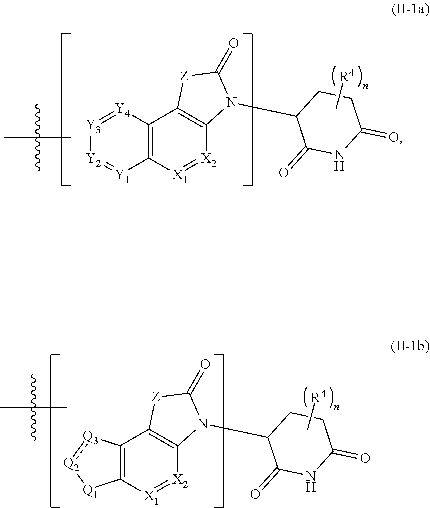
wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a single bond and a double bond; Q₁, Q₂, and Q₃ are independently selected from O, S, NH, CH₂, N, and CH, wherein the NH, CH₂, or CH is optionally substituted with R¹; Z, R¹, R², R⁴, and n are as defined in claim 1.

7. The compound represented by formula (I) or the pharmaceutically acceptable salt thereof according to claim 1, wherein R¹ and R² are independently selected from halogen, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}; or
wherein R⁵ is independently selected from halogen, ═O, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}; or
wherein R³ is independently selected from H, halogen, CN, OH, NH₂, C₁-C₁₀ alkyl, C₂-C₁₀ alkenyl, C₂-C₁₀ alkynyl, and C₃-C₁₀ cycloalkyl, wherein the OH, NH₂, C₁-C₁₀ alkyl, C₂-C₁₀ alkenyl, C₂-C₁₀ alkynyl, or C₃-C₁₀ cycloalkyl is optionally substituted with R^{a}; or
wherein R¹ and R³, together with the atoms linked thereto, form 5-, 6-, 7-, or 8-membered heterocyclic ring, wherein the 5-, 6-, 7-, or 8-membered heterocyclic ring is optionally substituted with R⁵; or
wherein each R⁴ is independently selected from halogen, CN, OH, NH₂, and C₁-C₆ alkyl, wherein the OH, NH₂, or C₁-C₆ alkyl is optionally substituted with R^{a}; or
wherein each R^{a} is independently selected from halogen, CN, OH, NH₂, C₁-C₁₀ alkyl, C₂-C₁₀ alkenyl, C₂-C₁₀ alkynyl, C₃-C₁₀ cycloalkyl, and 4- to 8-membered heterocyclyl, wherein the C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c}; or
wherein m and P are independently selected from 0, 1, 2, 3, and 4; or
wherein n is selected from 0 and 1.

8.-14. (canceled)

15. The compound represented by formula (I) or the pharmaceutically acceptable salt thereof according to claim 1, wherein L is selected from 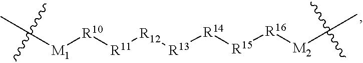
wherein M₁ and M₂ are independently selected from bond, —NR²⁰—, —C(O)—, —C(O)O—, —SO₂—, —S(O)—, —O—, —S—, —C(═S)—, —C(O)NR²⁰—, —NR²⁰C(O)O—, —NR²⁰S(O)₂—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹;
R¹⁰, R¹¹, R¹², R¹³, R¹⁴, R¹⁵, and R¹⁶ are independently selected from bond, —(O—CH₂CH₂)ₖ—, —C(O)—, —C(O)O—, —SO₂—, —S(O)—, —O—, —S—, —C(S)—, —C(═NR²⁰)—, —C(O)NR²⁰—, —NR²⁰—, —NR²⁰C(O)O—, —NR²⁰S(O)₂—, —P(O)R²⁰—, —P(O)(OR²⁰)O—, —P(O)(OR²⁰)—, 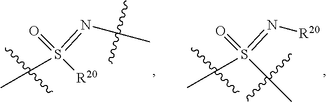
2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹;
k is independently selected from 1, 2, 3, 4, 5, and 6;
R²⁰ is selected from H, halogen, CN, OH, NH₂, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{f};
R²¹ is selected from halogen, CN, OH, NH₂, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{f};
each R^{f} is independently selected from halogen, CN, OH, NH₂, and C₁-C₆ alkyl.

16. The compound represented by formula (I) or the pharmaceutically acceptable salt thereof according to claim 15, wherein L is selected from 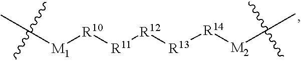
wherein M₁, M₂, R¹⁰, R¹¹, R¹², R¹³, and R¹⁴ are as defined in claim 15; or
wherein L is selected from 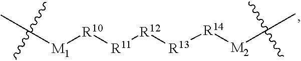
wherein R¹⁰, R¹¹, R¹², R¹³, and R¹⁴ are independently selected from bond, —(O—CH₂CH₂)ₖ—, —C(O)—, —C(O)O—, —O—, —C(O)NR²⁰—, —NR²⁰—, —NR²⁰C(O)O—, —NR²⁰S(O)₂—, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the C₁-C₁₀ alkylene, C₂-C₁₀ alkenylene, C₂-C₁₀ alkynylene, 4- to 9-membered heterocyclylene, C₆-C₁₀ arylene, or 5- to 10-membered heteroarylene is optionally substituted with R²¹; M₁, M₂, R²⁰, R²¹, and k are as defined in claim 15; or
wherein L is selected from 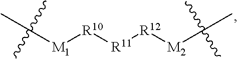
wherein M₁, M₂, R¹⁰, R¹¹, and R¹² are as defined in claim 15.

17. (canceled)

18. (canceled)

19. The compound represented by formula (I) or the pharmaceutically acceptable salt thereof according to claim 15, wherein M₁ and M₂ are independently selected from bond, —NR²⁰—, —C(O)—, —C(O)O—, —O—, —S—, —C(O)NR²⁰—, —NR²⁰C(O)O—, —NR²⁰S(O)₂—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹, wherein R²⁰ and R²¹ are as defined in claim 15; or
wherein R¹⁰, R¹¹, R¹², R¹³, R¹⁴, R¹⁵, and R¹⁶ are independently selected from bond, —(O—CH₂CH₂)ₖ—, —C(O)—, —C(O)O—, —SO₂—, —S(O)—, —O—, —S—, —C(O)NR²⁰—, —NR²⁰—, —NR²⁰C(O)O—, —NR²⁰S(O)₂—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R²¹, wherein k, R²⁰, and R²¹ are as defined in claim 15.

20. (canceled)

21. The compound represented by formula (I) or the pharmaceutically acceptable salt thereof according to claim 1, wherein PTM is selected from binding moieties of the following targeted proteins: ALK, AR, BET1, BRAF, BRCA2, BRD4, BRD9, BTK, BRM, CBL, CCNE1, CCNE2, CCR4, CCR7, CCR9, CD47, CLDN18, CYP, DDR1, DMPK, EGFR, ERBB2, ERBB3, ERBB4, FGFR1, FGFR2, FGFR3, FGFR4, GSPT1, JAK1, JAK3, KIF18A, KRAS, LCK, MET, NTRK1, NTRK2, NTRK3, PCSK9, PKMYT1, PARP7, PARP14, RAD51, RBM10, RET, RORA, STAT3, SOS1, TYK2, USP1, and USP14.

22. The compound represented by formula (I) or the pharmaceutically acceptable salt thereof according to claim 1, wherein the compound represented by formula (I) or the pharmaceutically acceptable salt is selected from:

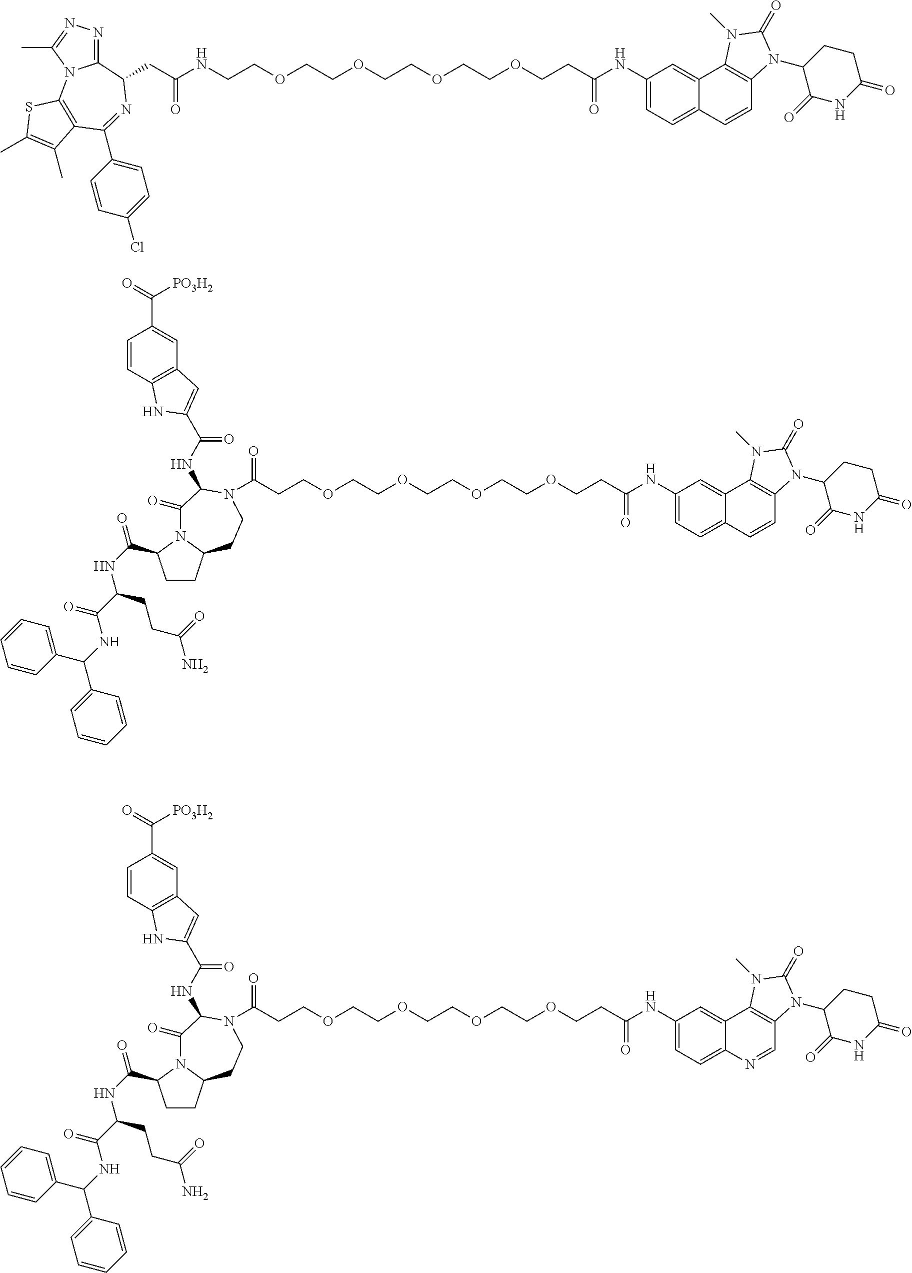

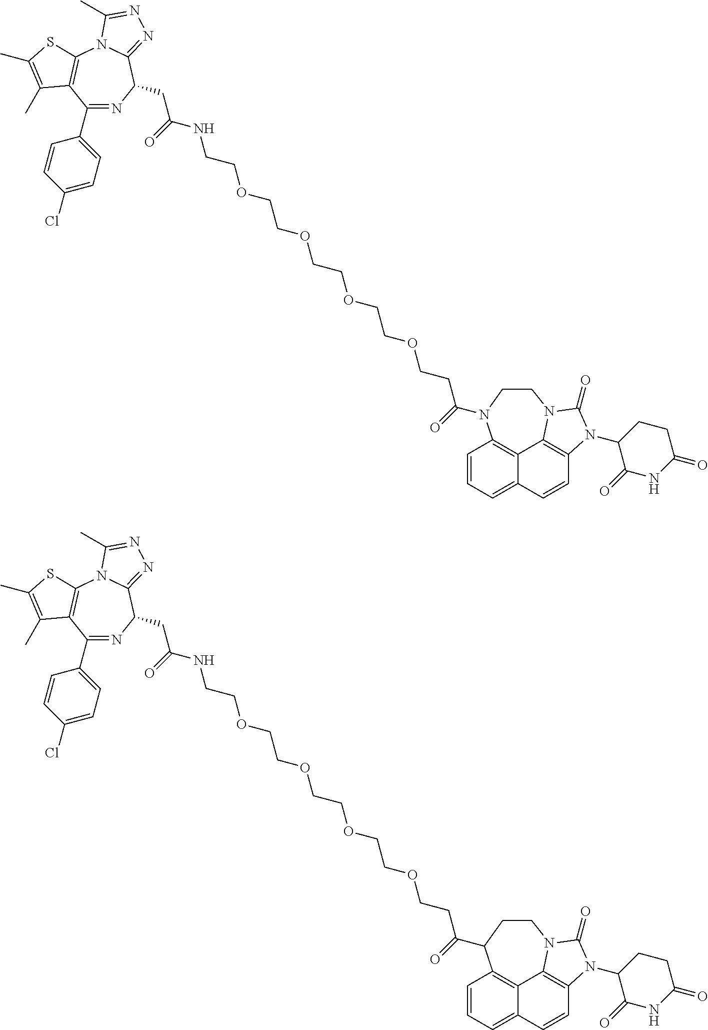 [SMILES: Cc1sc2c(c1C)C(c1ccc(Cl)cc1)=N[C@@H](CC(=O)NCCOCCOCCOCCOCCC(=O)N1CCn3c(=O)n(C4CCC(=O)NC4=O)c4ccc5cccc1c5c43)c1nnc(C)n1-2 ; Cc1sc2c(c1C)C(c1ccc(Cl)cc1)=N[C@@H](CC(=O)NCCOCCOCCOCCOCCC(=O)C1CCn3c(=O)n(C4CCC(=O)NC4=O)c4ccc5cccc1c5c43)c1nnc(C)n1-2]

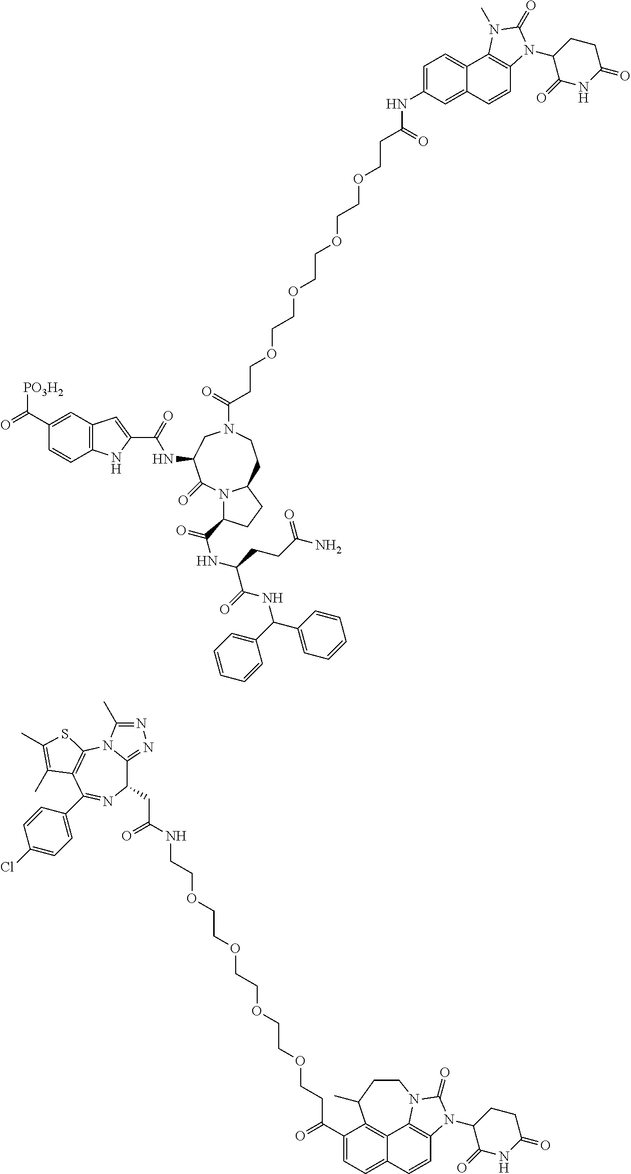

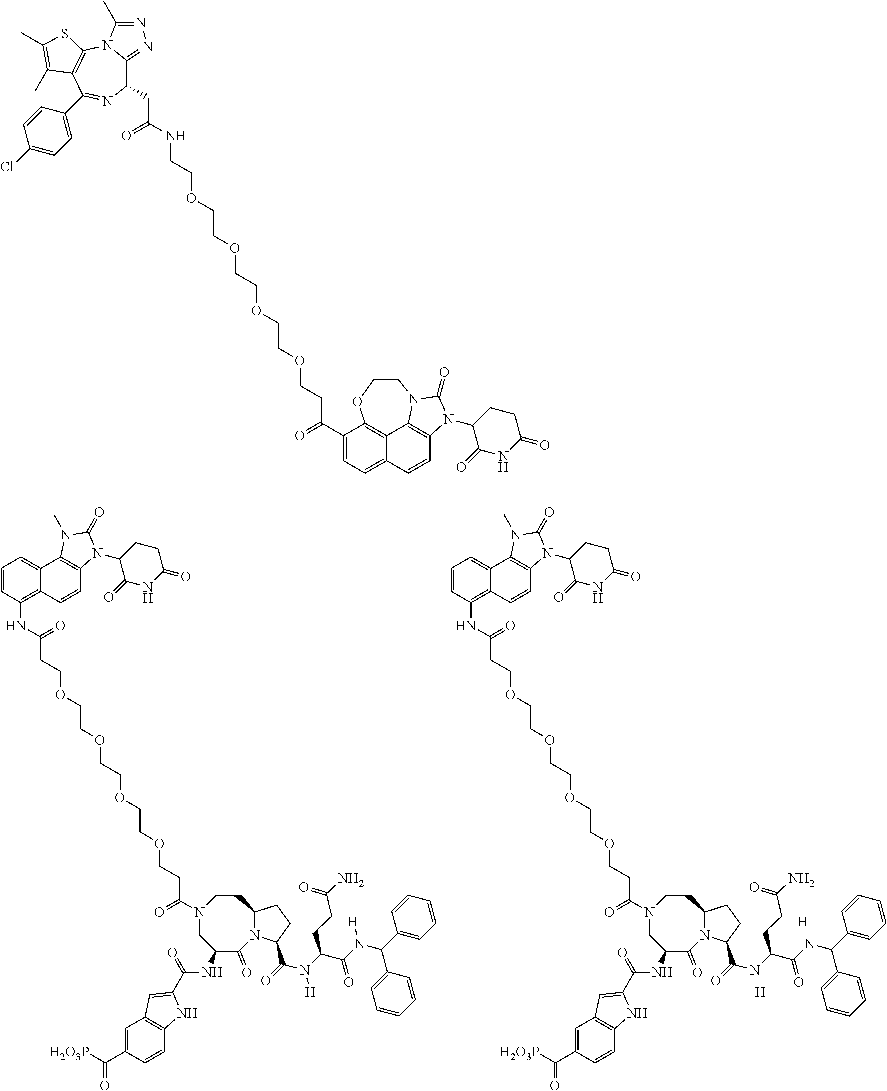

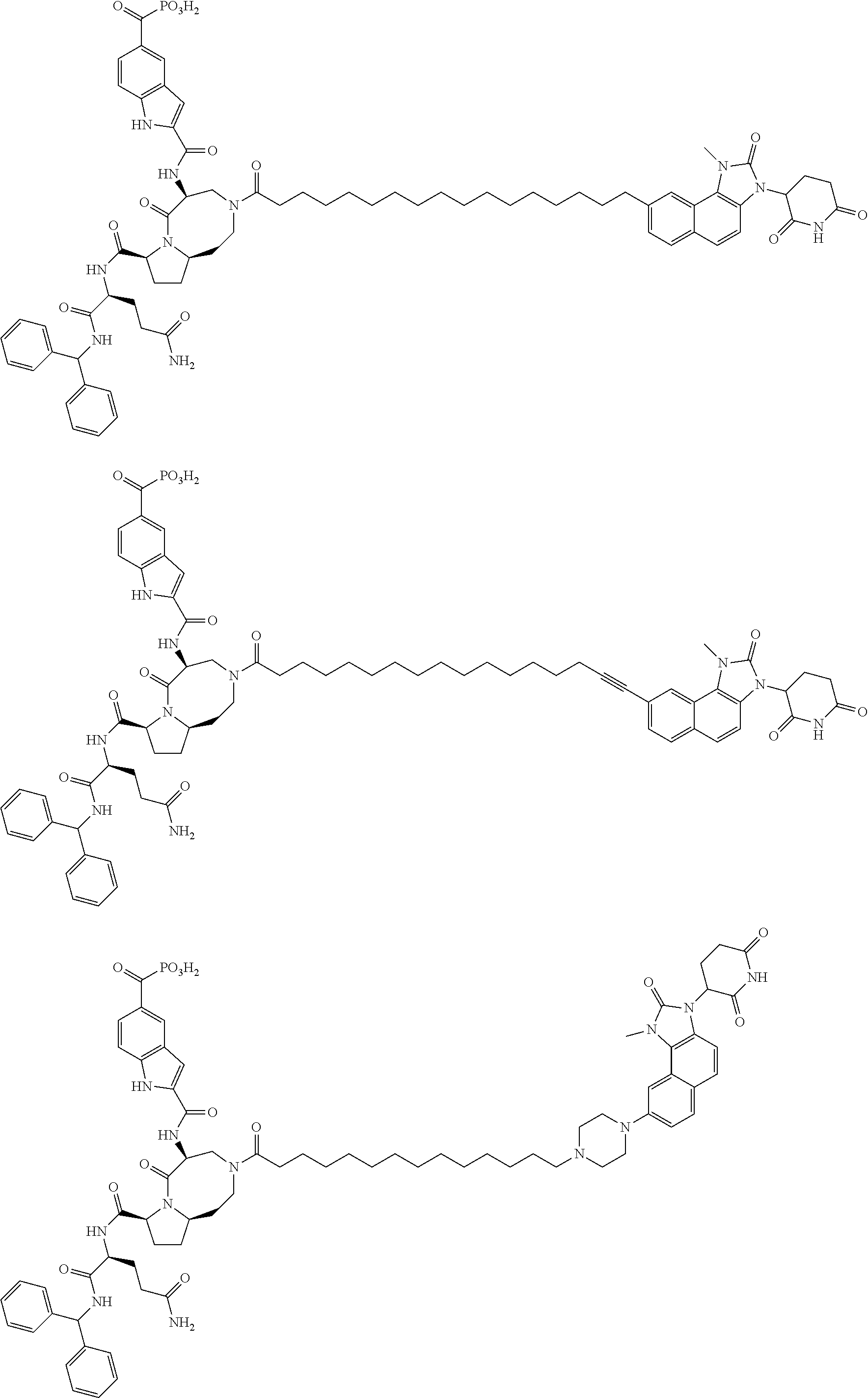

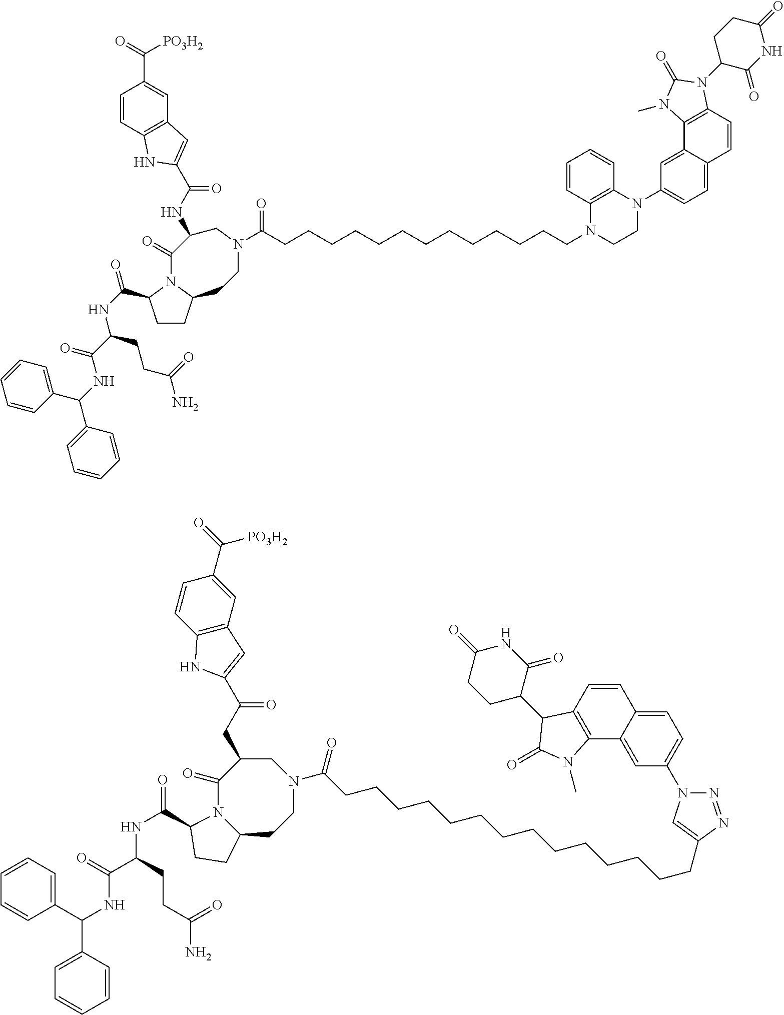

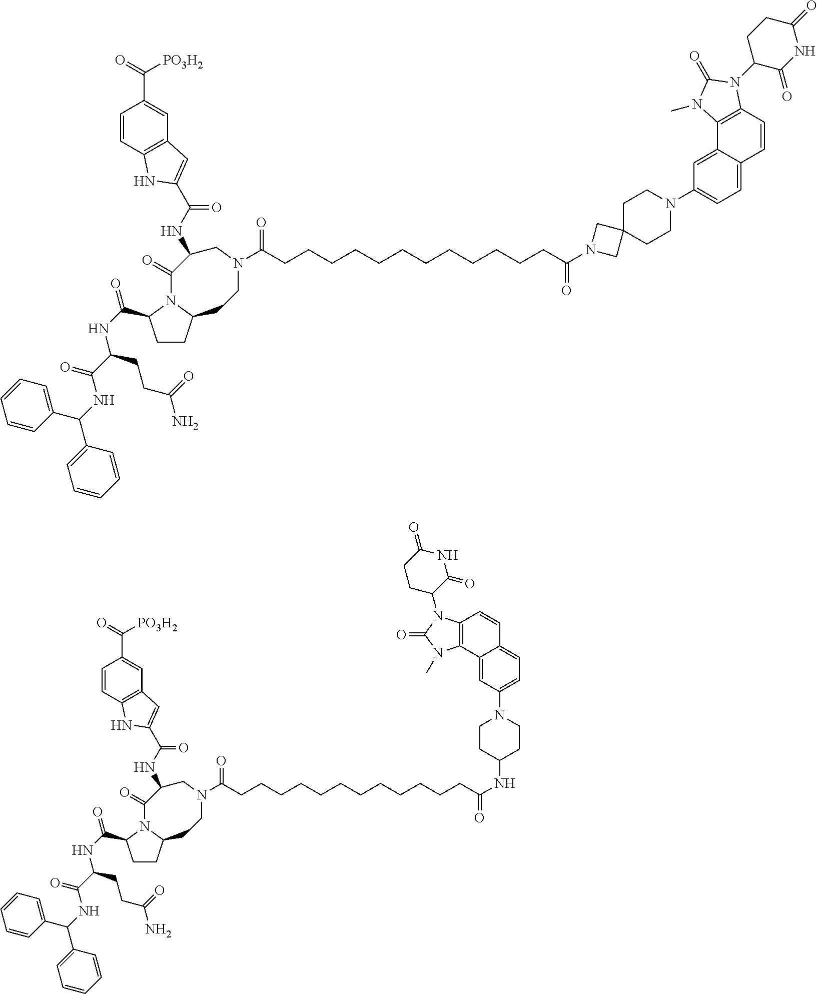

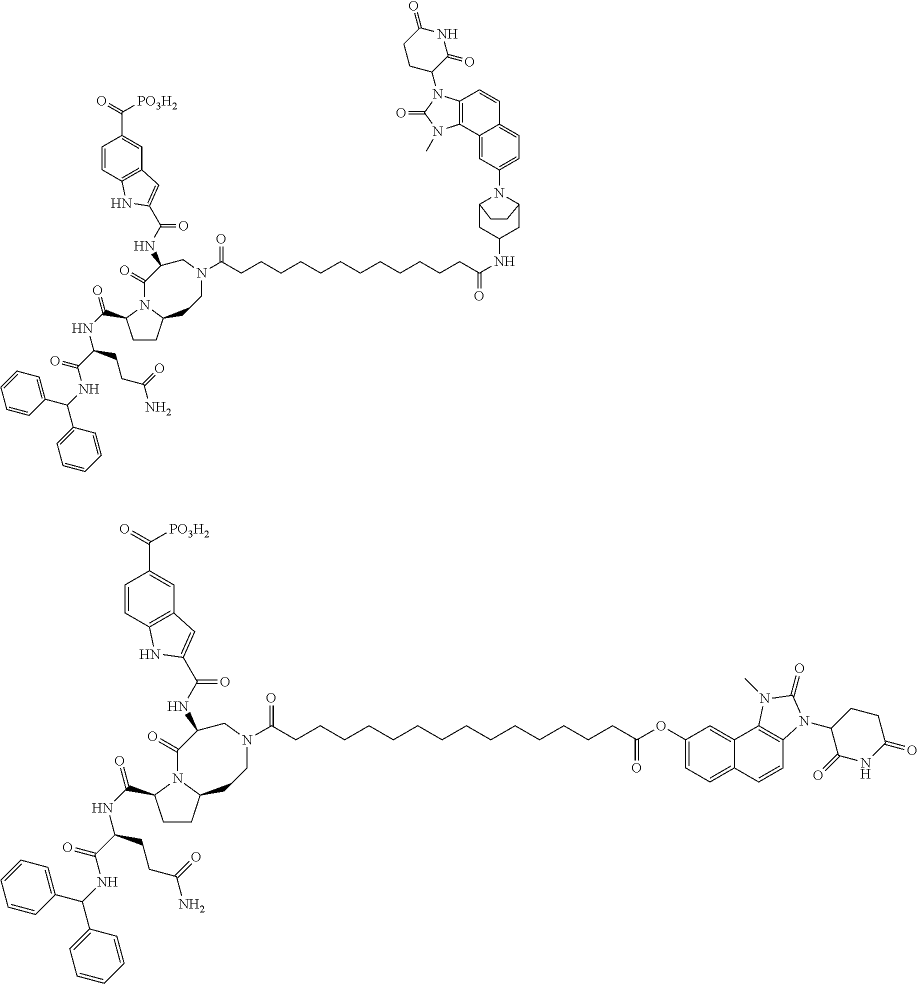

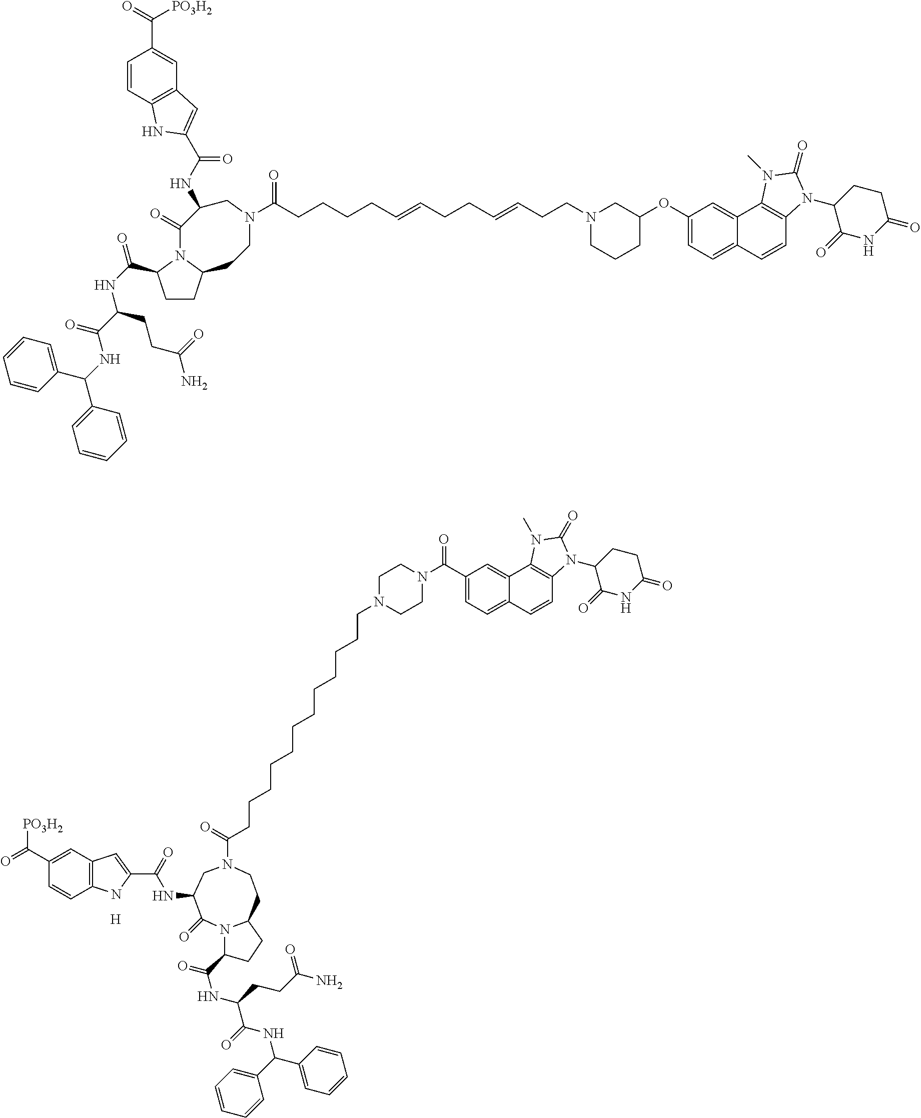

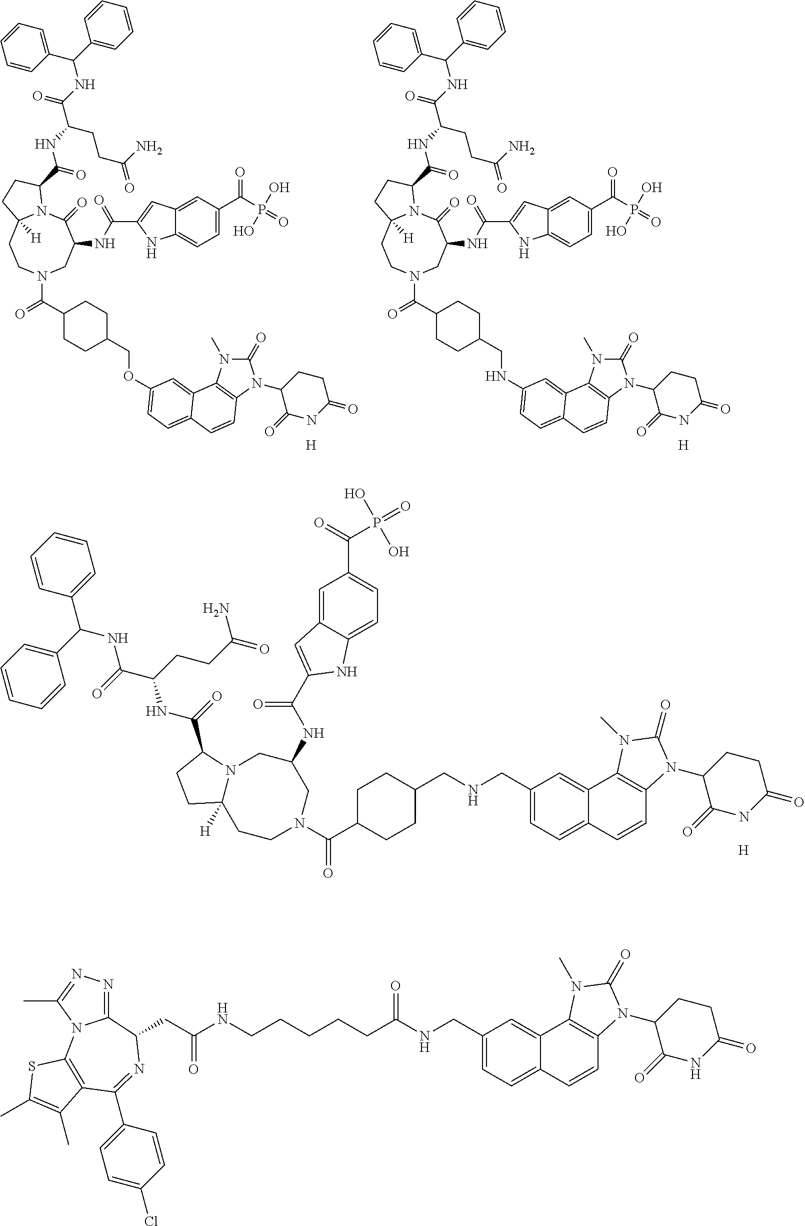

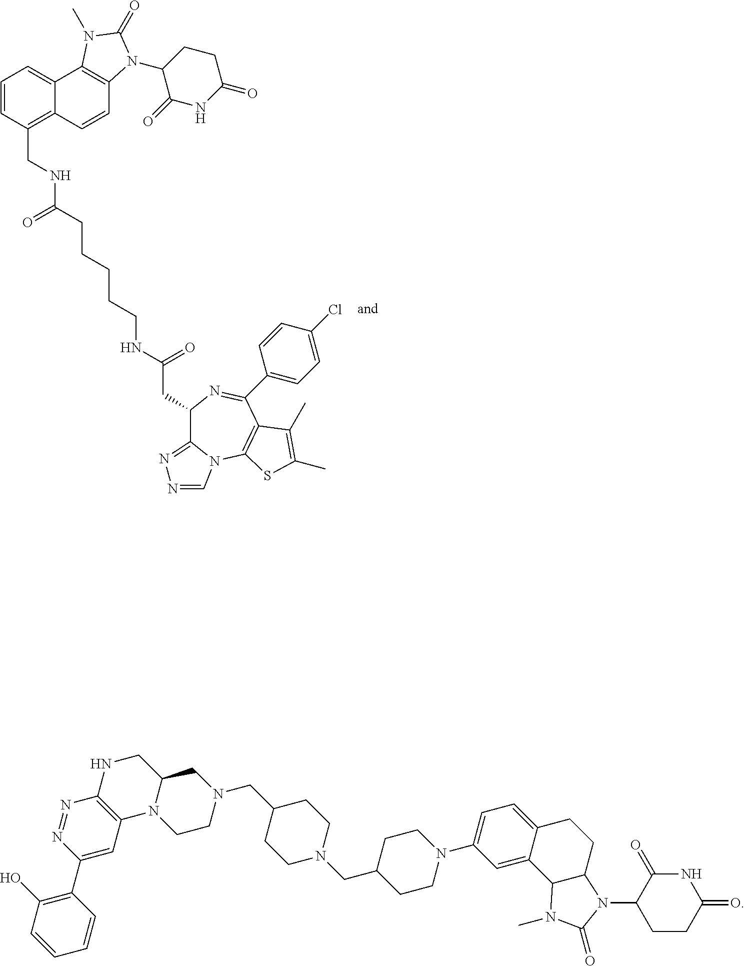 [SMILES: Cc1sc2c(c1C)C(c1ccc(Cl)cc1)=N[C@@H](CC(=O)NCCCCCC(=O)NCc1cccc3c1ccc1c3n(C)c(=O)n1C1CCC(=O)NC1=O)c1nncn1-2 ; CN1C(=O)N(C2CCC(=O)NC2=O)C2CCc3ccc(N4CCC(CN5CCC(CN6CCN7c8cc(-c9ccccc9O)nnc8NC[C@H]7C6)CC5)CC4)cc3C21]

23. A pharmaceutical composition, comprising the compound represented by formula (I) or the pharmaceutically acceptable salt thereof according to claim 1 and a pharmaceutically acceptable excipient.

24. (canceled)

25. (canceled)

26. A method for treating an abnormal cell proliferation disease in a mammal, comprising administering to a mammal in need of the treatment a therapeutically effective amount of the compound represented by formula (I) or the pharmaceutically acceptable salt thereof according to claim 1.

27. The method according to claim 26, wherein the abnormal cell proliferation disease is cancer.

28. A compound represented by formula (III) or a pharmaceutically acceptable salt thereof: 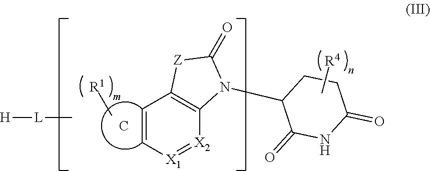
wherein X₁ and X₂ are independently selected from N and CH, wherein the CH is optionally substituted with R²;
Z is selected from C(R³)₂, NR³, and O;
R¹, R², and R⁵ are each independently selected from halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
each R³ is independently selected from H, halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
or R¹ and R³, together with the atoms linked thereto, form C₅-C₈ saturated or partially saturated carbon ring, 5- to 6-membered heteroaromatic ring, or 5- to 8-membered heterocyclic ring, wherein the C₅-C₈ saturated or partially saturated carbon ring, 5- to 6-membered heteroaromatic ring, or 5- to 8-membered heterocyclic ring is optionally substituted with R⁵;
each R⁴ is independently selected from halogen, CN, NO₂, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₂-C₁₀ alkenyl, C₂-C₁₀ alkynyl, C₃-C₁₀ cycloalkyl, and 4- to 8-membered heterocyclyl, wherein the 2- to 10-membered heteroalkyl, C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and P are independently selected from 0, 1, 2, 3, 4, 5, and 6;
L represents a linking unit;
ring C is selected from 5- to 6-membered heteroaromatic ring and benzene ring;
provided that the following compounds or pharmaceutically acceptable salts thereof are excluded:

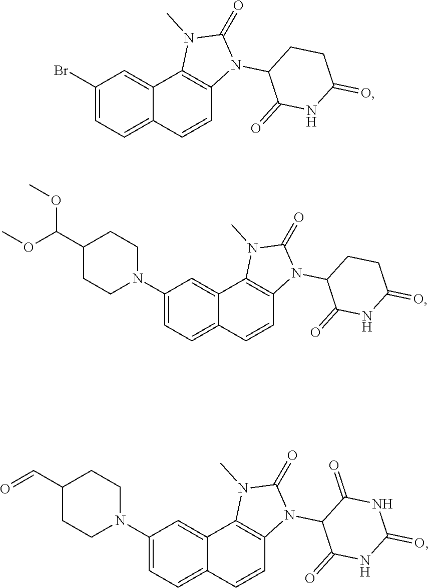 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

29. The compound represented by formula (III) or the pharmaceutically acceptable salt thereof according to claim 28, wherein the compound represented by formula (III) or the pharmaceutically acceptable salt thereof is selected from a compound represented by formula (III-1a) or formula (III-1b) or a pharmaceutically acceptable salt thereof: 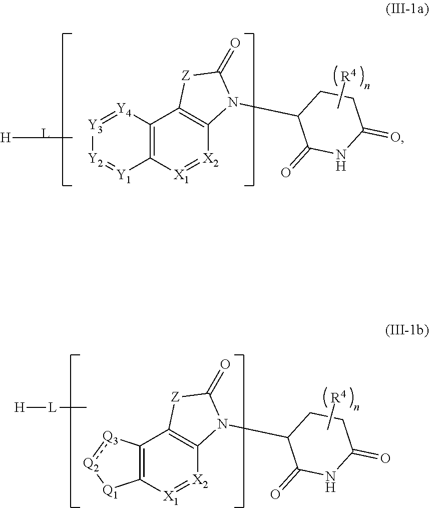
wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a double bond; Q₁, Q₂, and Q₃ are independently selected from O, S, NH, CH₂, N, and CH, wherein the NH, CH₂, or CH is optionally substituted with R¹; Z, R¹, R², R⁴, n, and L are as defined in claim 28;
provided that the following compounds or pharmaceutically acceptable salts thereof are excluded:

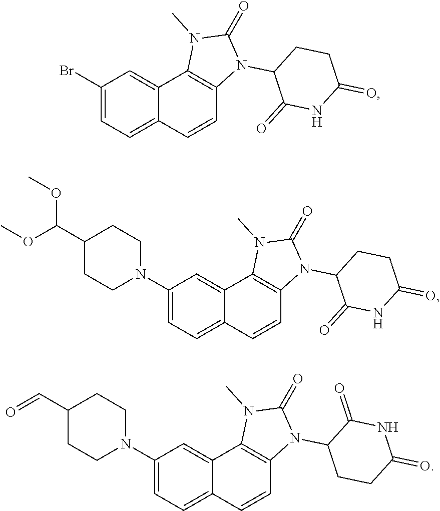 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2CCC(=O)NC2=O)c2ccc3ccc(N4CCC(C=O)CC4)cc3c21]

30. A compound represented by formula (IV) or a pharmaceutically acceptable salt thereof: 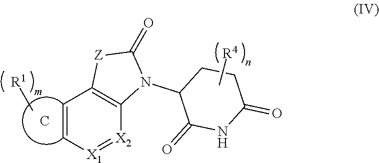
wherein X₁ and X₂ are independently selected from N and CH, wherein the CH is optionally substituted with R²;
Z is selected from C(R³)₂, NR³, and O;
R¹, R², and R⁵ are each independently selected from halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
each R³ is independently selected from H, halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
or R¹ and R³, together with the atoms linked thereto, form C₅-C₈ saturated or Partially saturated carbon ring, 5- to 6-membered heteroaromatic ring, or 5- to 8-membered heterocyclic ring, wherein the C₅-C₈ saturated or partially saturated carbon ring, 5- to 6-membered heteroaromatic ring, or 5- to 8-membered heterocyclic ring is optionally substituted with R⁵;
each R⁴ is independently selected from halogen, CN, NO₂, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₂-C₁₀ alkenyl, C₂-C₁₀ alkynyl, C₃-C₁₀ cycloalkyl, and 4- to 8-membered heterocyclyl, wherein the 2- to 10-membered heteroalkyl, C₁-C₁₀ alkyl, C₂-C₁₀ alkenyl, C₂-C₁₀ alkynyl, C₃-C₁₀ cycloalkyl, or 4- to 8-membered heterocyclyl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and P are independently selected from 0, 1, 2, 3, 4, 5, and 6;
ring C is selected from 5- to 6-membered heteroaromatic ring and benzene ring;
provided that the following compounds or pharmaceutically acceptable salts thereof are excluded:

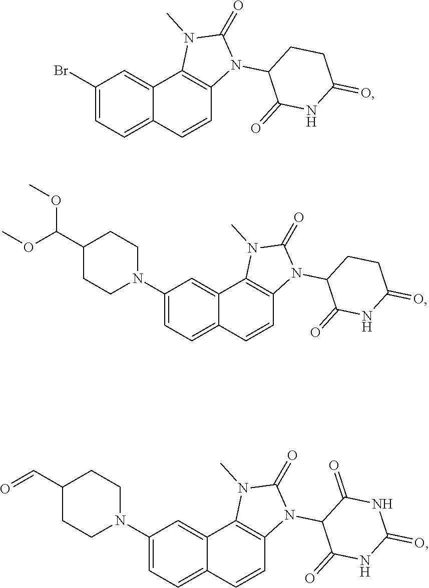 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2C(=O)NC(=O)NC2=O)c2ccc3ccc(N4CCC(C=O)CC4)cc3c21]

31. The compound represented by formula (IV) or the pharmaceutically acceptable salt thereof according to claim 30, wherein the compound represented by formula (IV) or the pharmaceutically acceptable salt thereof is selected from a compound represented by formula (IV-1a) or formula (IV-1 b) or a pharmaceutically acceptable salt thereof: 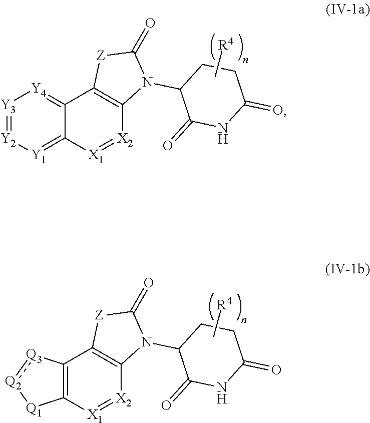
wherein X₁ and X₂ are independently selected from N and CH, wherein the CH is optionally substituted with R²; Y₁, Y₂, Y₃, and Y₄ are independently selected from N and CH, wherein the CH is optionally substituted with R¹; “ [symbol] ” is selected from a double bond; Q₁, Q₂, and Q₃ are independently selected from O, S, NH, CH₂, N, and CH, wherein the NH, CH₂, or CH is optionally substituted with R¹; Z, R¹, R², R⁴, and n are as defined in claim 30;
provided that the following compounds or pharmaceutically acceptable salts thereof are excluded:

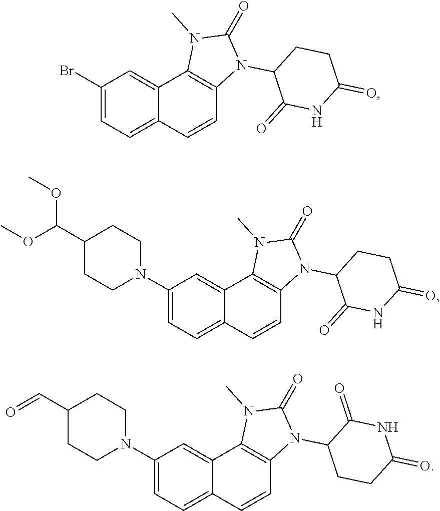 [SMILES: Cn1c(=O)n(C2CCC(=O)NC2=O)c2ccc3ccc(Br)cc3c21 ; COC(OC)C1CCN(c2ccc3ccc4c(c3c2)n(C)c(=O)n4C2CCC(=O)NC2=O)CC1 ; Cn1c(=O)n(C2CCC(=O)NC2=O)c2ccc3ccc(N4CCC(C=O)CC4)cc3c21]

32. (canceled)

33. The compound represented by the formula (IV) or a pharmaceutically acceptable salt thereof according to claim 30, selected from the following compounds or pharmaceutically acceptable salts thereof:

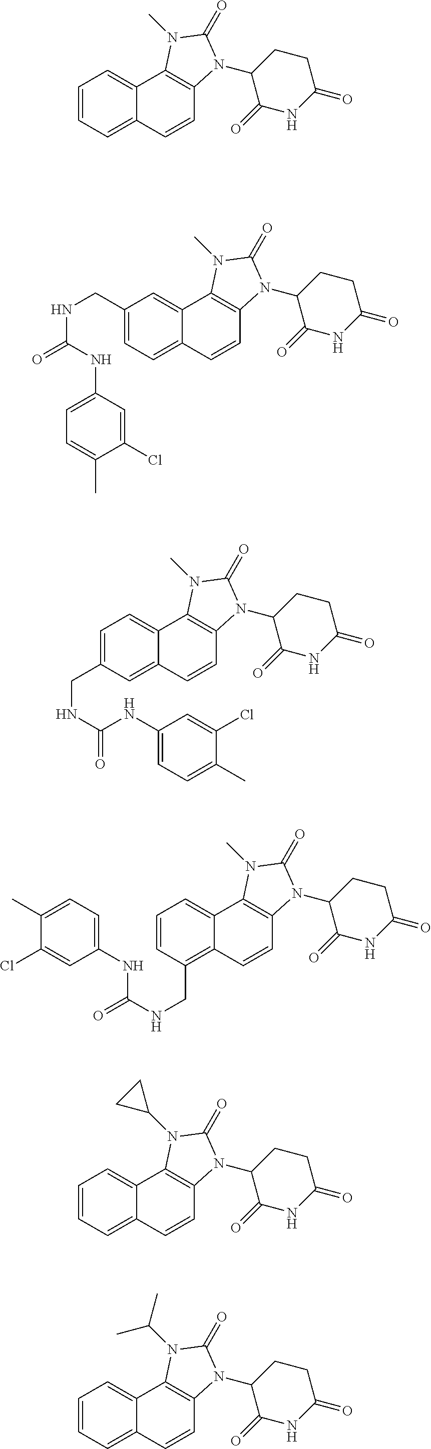 [SMILES: Cn1c(=O)n(C2CCC(=O)NC2=O)c2ccc3ccccc3c21 ; Cc1ccc(NC(=O)NCc2ccc3ccc4c(c3c2)n(C)c(=O)n4C2CCC(=O)NC2=O)cc1Cl ; Cc1ccc(NC(=O)NCc2ccc3c(ccc4c3n(C)c(=O)n4C3CCC(=O)NC3=O)c2)cc1Cl ; Cc1ccc(NC(=O)NCc2cccc3c2ccc2c3n(C)c(=O)n2C2CCC(=O)NC2=O)cc1Cl ; O=C1CCC(n2c(=O)n(C3CC3)c3c4ccccc4ccc32)C(=O)N1 ; CC(C)n1c(=O)n(C2CCC(=O)NC2=O)c2ccc3ccccc3c21]

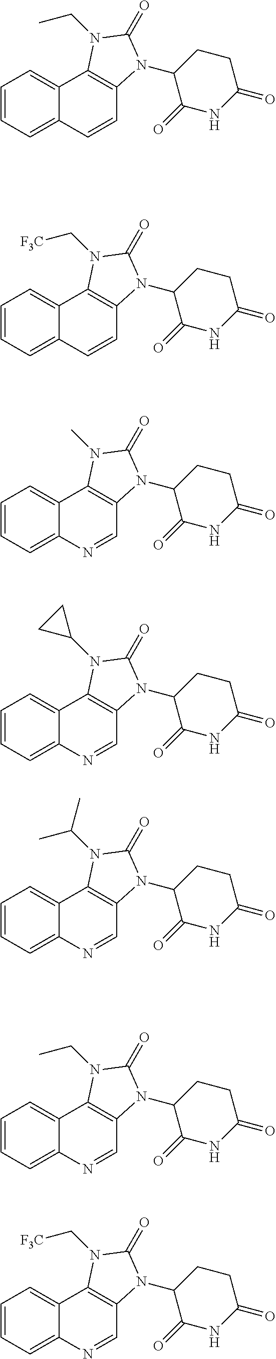

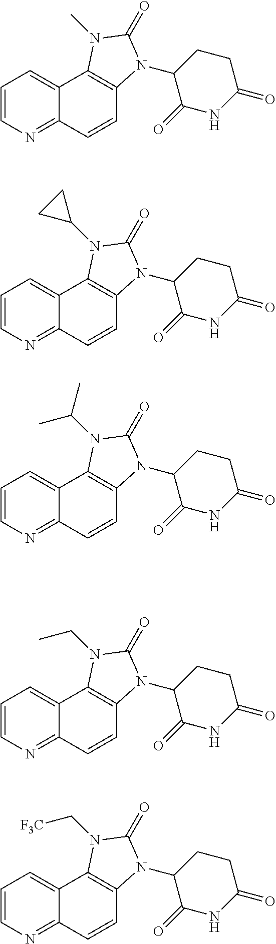

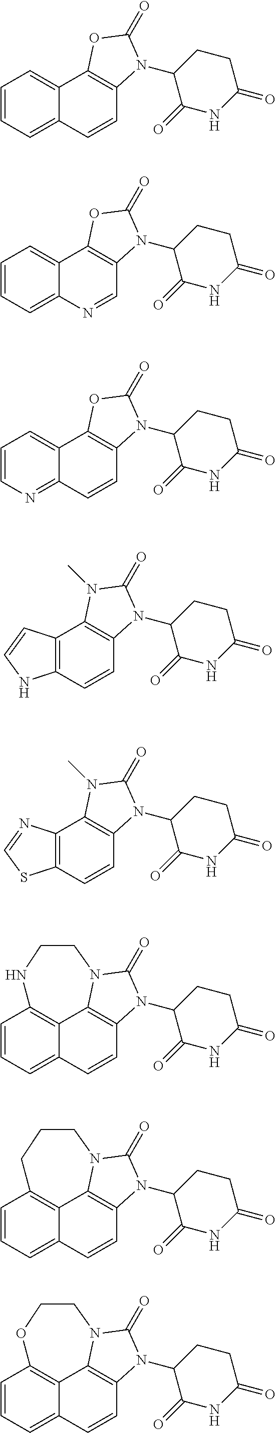

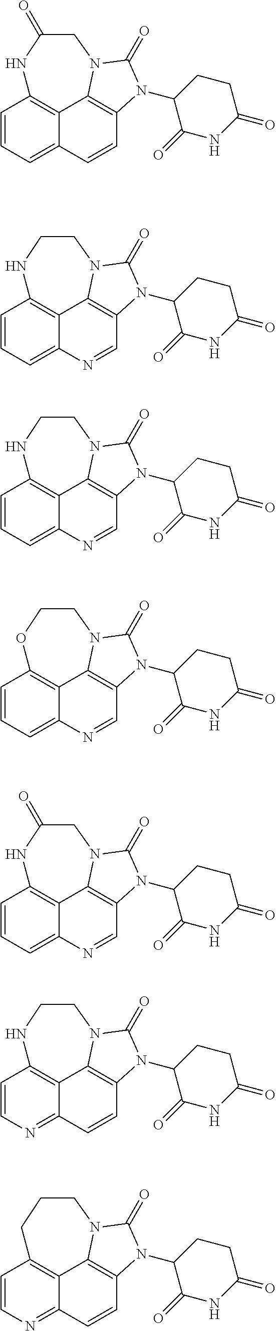

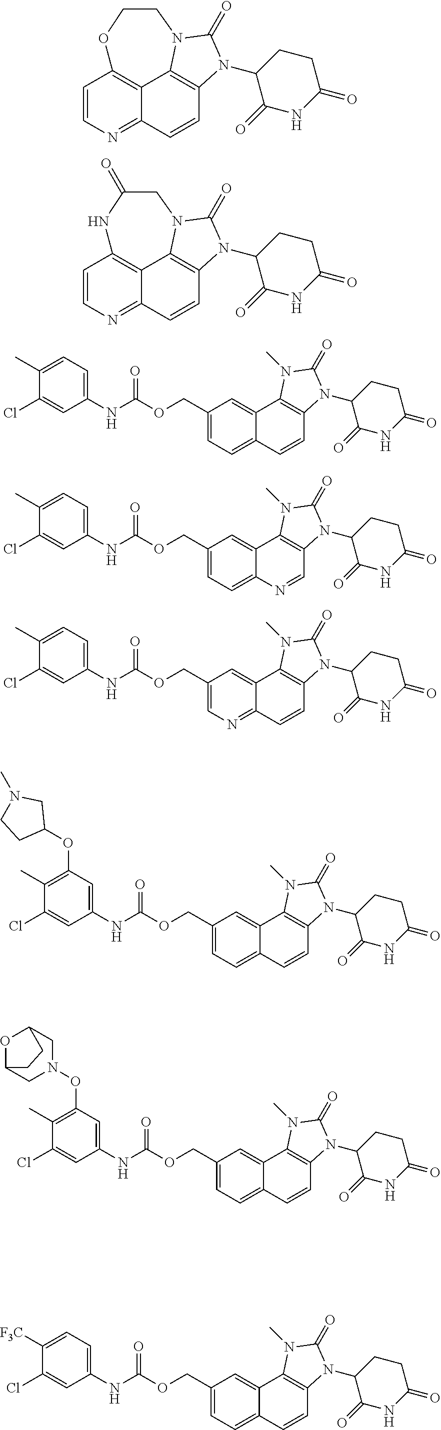

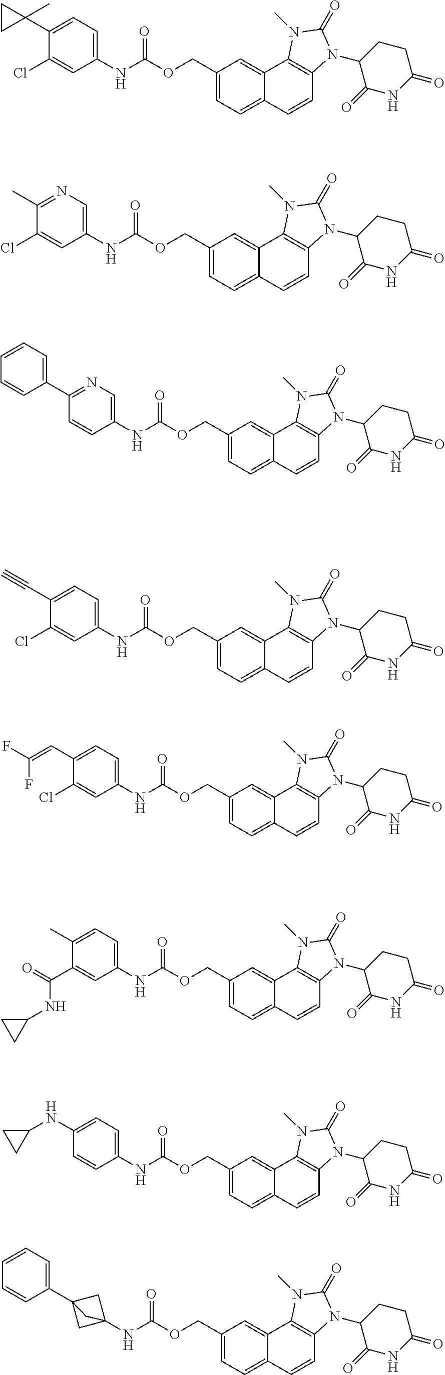

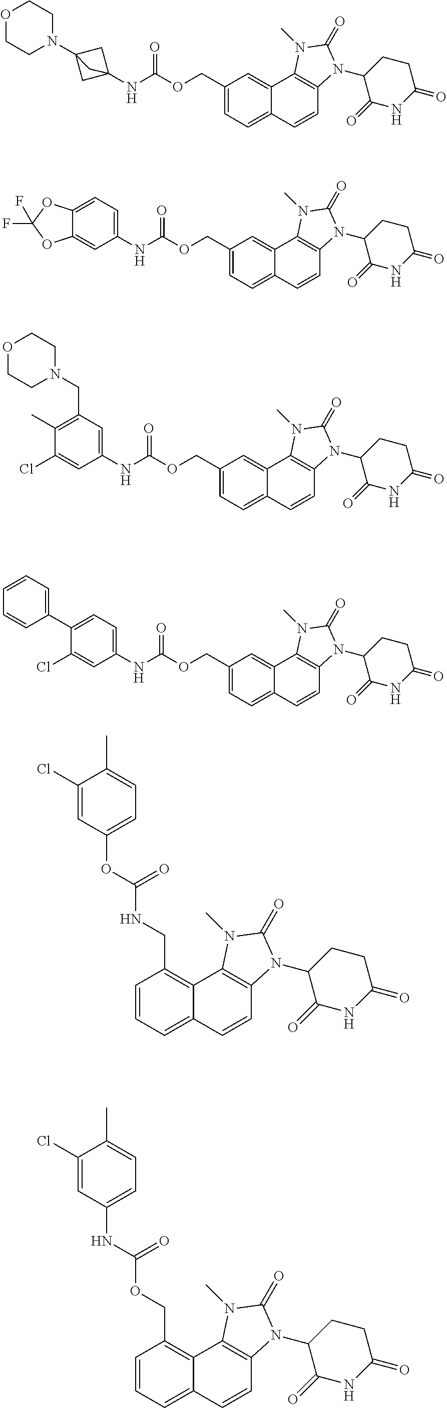 [SMILES: Cn1c(=O)n(C2CCC(=O)NC2=O)c2ccc3ccc(COC(=O)NC45CC(N6CCOCC6)(C4)C5)cc3c21 ; Cn1c(=O)n(C2CCC(=O)NC2=O)c2ccc3ccc(COC(=O)Nc4ccc5c(c4)OC(F)(F)O5)cc3c21 ; Cc1c(Cl)cc(NC(=O)OCc2ccc3ccc4c(c3c2)n(C)c(=O)n4C2CCC(=O)NC2=O)cc1CN1CCOCC1 ; Cn1c(=O)n(C2CCC(=O)NC2=O)c2ccc3ccc(COC(=O)Nc4ccc(-c5ccccc5)c(Cl)c4)cc3c21 ; Cc1ccc(OC(=O)NCc2cccc3ccc4c(c23)n(C)c(=O)n4C2CCC(=O)NC2=O)cc1Cl ; Cc1ccc(NC(=O)OCc2cccc3ccc4c(c23)n(C)c(=O)n4C2CCC(=O)NC2=O)cc1Cl]

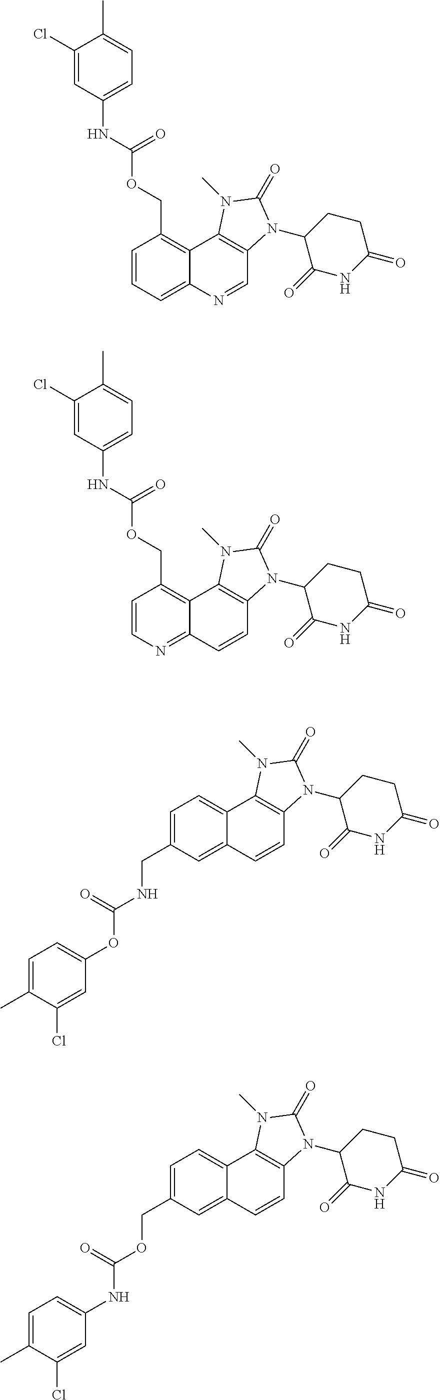 [SMILES: Cc1ccc(NC(=O)OCc2cccc3ncc4c(c23)n(C)c(=O)n4C2CCC(=O)NC2=O)cc1Cl ; Cc1ccc(NC(=O)OCc2ccnc3ccc4c(c23)n(C)c(=O)n4C2CCC(=O)NC2=O)cc1Cl ; Cc1ccc(OC(=O)NCc2ccc3c(ccc4c3n(C)c(=O)n4C3CCC(=O)NC3=O)c2)cc1Cl ; Cc1ccc(NC(=O)OCc2ccc3c(ccc4c3n(C)c(=O)n4C3CCC(=O)NC3=O)c2)cc1Cl]

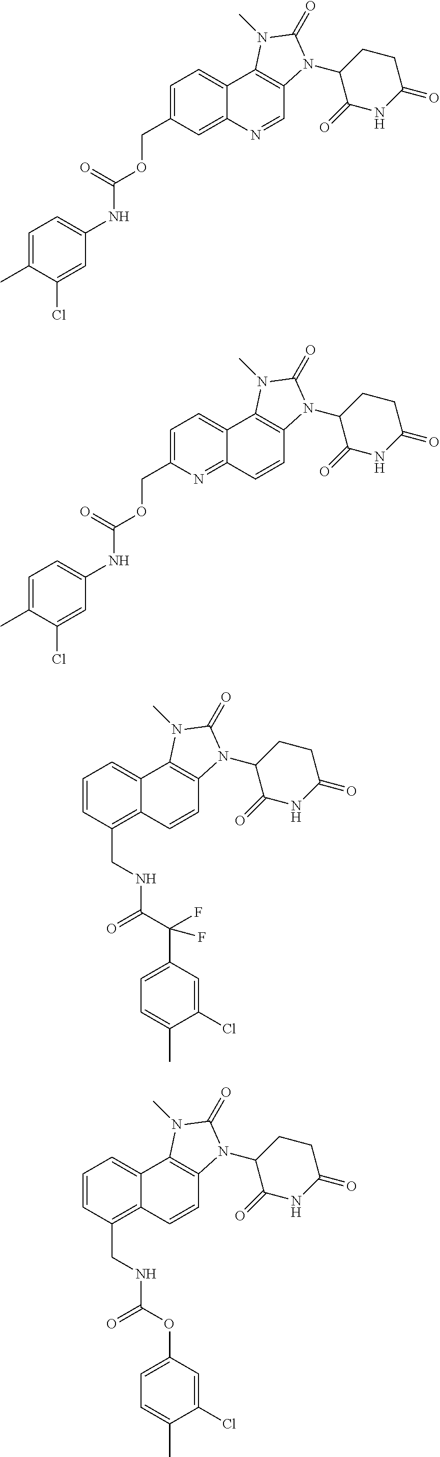

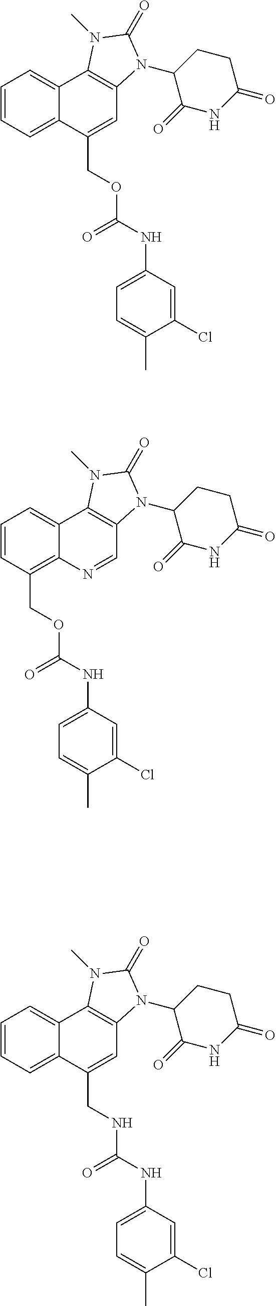 [SMILES: Cc1ccc(NC(=O)OCc2cc3c(c4ccccc24)n(C)c(=O)n3C2CCC(=O)NC2=O)cc1Cl ; Cc1ccc(NC(=O)OCc2cccc3c2ncc2c3n(C)c(=O)n2C2CCC(=O)NC2=O)cc1Cl ; Cc1ccc(NC(=O)NCc2cc3c(c4ccccc24)n(C)c(=O)n3C2CCC(=O)NC2=O)cc1Cl]

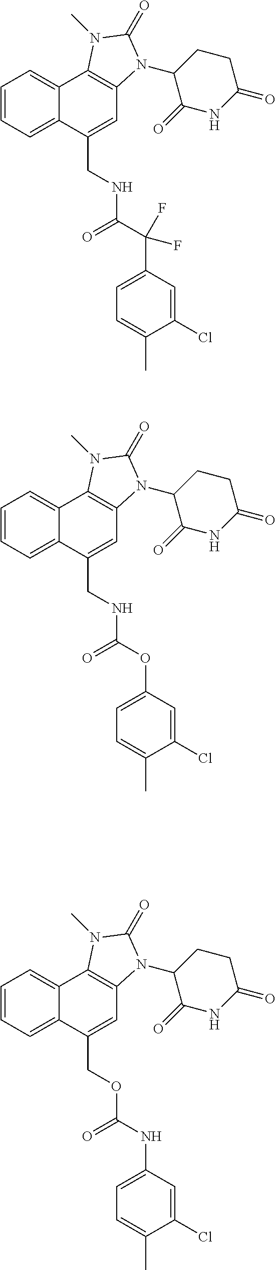 [SMILES: Cc1ccc(C(F)(F)C(=O)NCc2cc3c(c4ccccc24)n(C)c(=O)n3C2CCC(=O)NC2=O)cc1Cl ; Cc1ccc(OC(=O)NCc2cc3c(c4ccccc24)n(C)c(=O)n3C2CCC(=O)NC2=O)cc1Cl ; Cc1ccc(NC(=O)OCc2cc3c(c4ccccc24)n(C)c(=O)n3C2CCC(=O)NC2=O)cc1Cl]

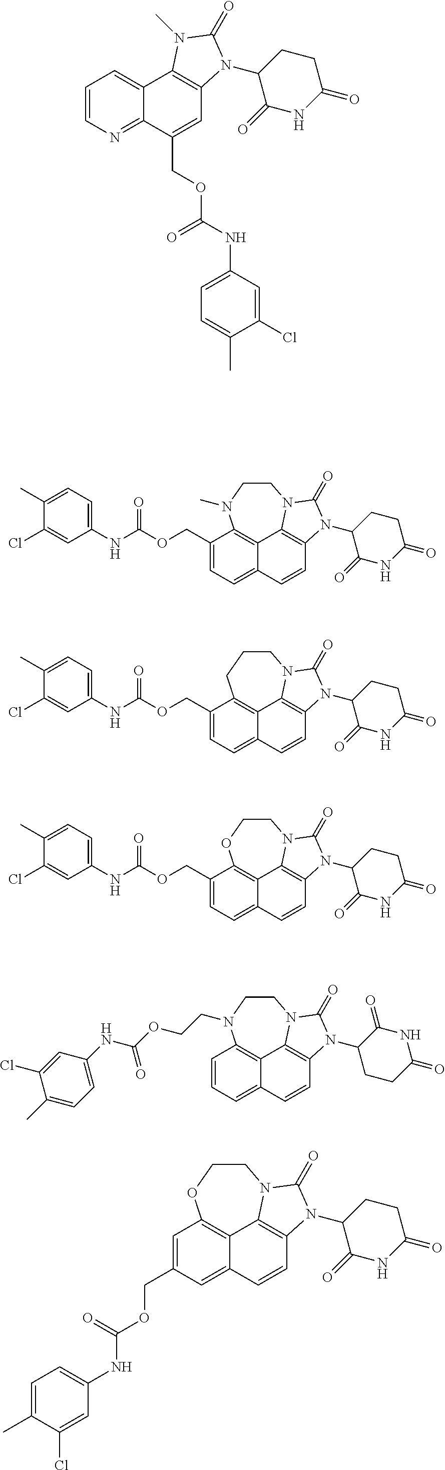 [SMILES: Cc1ccc(NC(=O)OCc2cc3c(c4cccnc24)n(C)c(=O)n3C2CCC(=O)NC2=O)cc1Cl ; Cc1ccc(NC(=O)OCc2ccc3ccc4c5c3c2N(C)CCn5c(=O)n4C2CCC(=O)NC2=O)cc1Cl ; Cc1ccc(NC(=O)OCc2ccc3ccc4c5c3c2CCCn5c(=O)n4C2CCC(=O)NC2=O)cc1Cl ; Cc1ccc(NC(=O)OCc2ccc3ccc4c5c3c2OCCn5c(=O)n4C2CCC(=O)NC2=O)cc1Cl ; Cc1ccc(NC(=O)OCCN2CCn3c(=O)n(C4CCC(=O)NC4=O)c4ccc5cccc2c5c43)cc1Cl ; Cc1ccc(NC(=O)OCc2cc3c4c(ccc5c4n(c(=O)n5C4CCC(=O)NC4=O)CCO3)c2)cc1Cl]

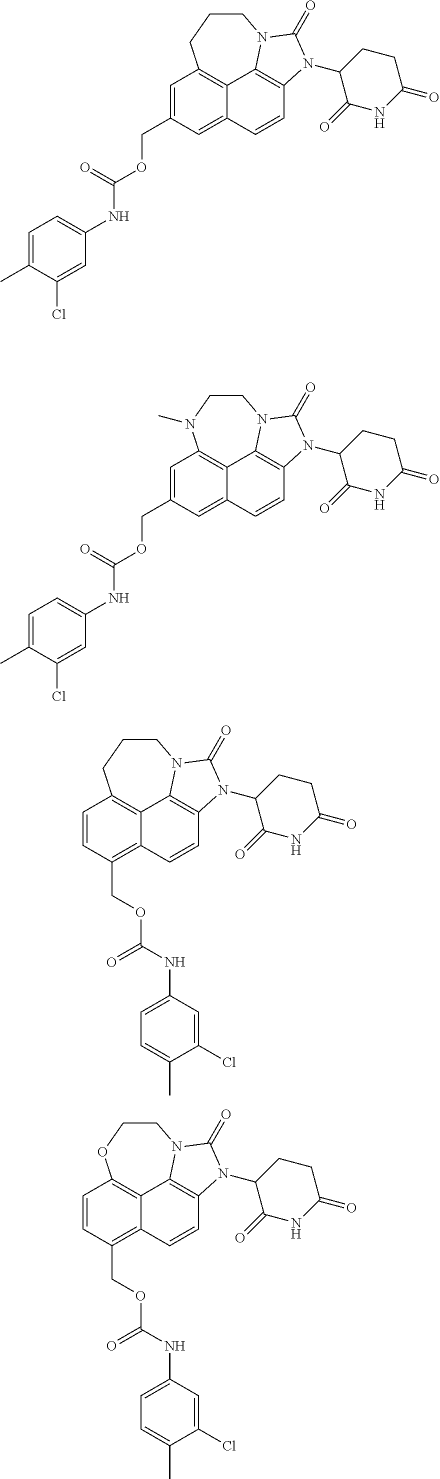

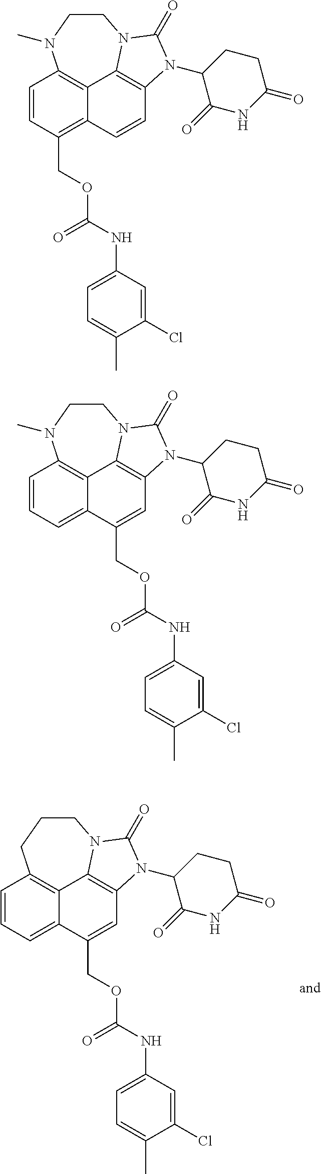

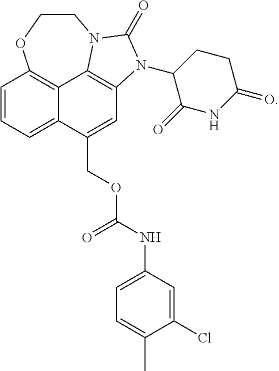 [SMILES: Cc1ccc(NC(=O)OCc2cc3c4c5c(cccc25)OCCn4c(=O)n3C2CCC(=O)NC2=O)cc1Cl]

34. (canceled)

35. (canceled)

36. A method for treating an abnormal cell proliferation disease in a mammal, comprising administering to a mammal in need of the treatment a therapeutically effective amount of the compound represented by formula (IV) or the pharmaceutically acceptable salt thereof according to claim 30.

37. The method according to claim 36, wherein the abnormal cell proliferation disease is cancer.
